(19) United States
(12) Patent Application Publication
(10) Pub. No.: US 2009/0246759 A1
(43) Pub. Date: Oct. 1, 2009

(54) TEST AND MODEL FOR INFLAMMATORY DISEASE

(75) Inventors: Nicholas J. Lench, Oxfordshire (GB); Maxine J. Allen, Oxfordshire (GB); Rosemary K. Nicholls, Oxfordshire (GB)

Correspondence Address: Workman Nydegger;1000 Eagle Gate Tower, 60 East South Temple, Salt Lake City, UT, 84111, US

(73) Assignee: MOLECULAR SKINCARE LIMITED, SHEFFIELD (GB)

(21) Appl. No.: 11/944,212

(22) Filed: Nov. 21, 2007

Related U.S. Application Data
(63) Continuation of application No. 10/467,752, filed on Feb. 4, 2004, now abandoned.

(30) Foreign Application Priority Data
Feb. 13, 2001 (GB) GB0103514.6

(51) Int. Cl. C12Q 1/68

(52) U.S. Cl. 435/6

(57) ABSTRACT

The present invention relates to a means and methods for determining susceptibility to SEEK1 mediated diseases, such as psoriasis. In addition, there is provided polynucleotides encoding the SEEK1 protein having one or more nucleotide insertions, deletions or substitutions at one or novel positions, and the SEEK1 protein having one or more amino acid insertions, deletions and substitutions. Host cells and transgenics non-human animals comprising polynucleotides or proteins of the invention are also provided. Methods of screening for agents for use in treating SEEK1 mediated disease are also provided.

This patent application is a continuation of U.S. patent application Ser. No. 10/467,752, filed Feb. 4, 2004, which is a 371 nationalization of PCT/GB02/00653, filed Feb. 13, 2002, which claims priority to U.K. patent application having serial number GB 0103514.6, filed Feb. 13, 2001 which foregoing patent application are incorporated herein by specific reference in their entirety.

The present invention relates to polymorphisms in the SEEK1 gene and protein, and the exploitation of these polymorphisms in the detection and/or treatment of SEEK1 mediated disease, for example inflammatory diseases including psoriasis. The present invention also relates to polynucleotides encoding the SEEK1 protein, and having one or more nucleotide polymorphisms, and to a protein encoded by said polynucleotides. Also provided are transgenic non-human animals comprising the polynucleotides of the present invention; and methods and kits for treating, diagnosing or determining susceptibility to SEEK1 mediated disease, in particular by way of gene therapy.

In recent years, it has been recognised that there is considerable genetic diversity in human populations, with common polymorphisms occurring on average at least every kilobase in the genome. Polymorphisms which affect gene expression or activity of the encoded gene product may account for susceptibility to, or expression of, disease conditions, either directly or through interaction with other genetic and environmental factors.

Understanding the molecular basis for disease, by sequencing the human genome and characterising polymorphisms, will enable the identification of those individuals at greatest risk of disease. This will allow the better matching of treatment and disease, and enable the production of new and improved targets for drugs. Screening and treatment of disease may also be better targeted to those in need, thus increasing the cost-effectiveness of health-care provision.

One area in need of such approaches is the diagnosis and treatment of inflammatory diseases. Inflammation, which can be broadly defined as the destructive sequelae to activation of elements of the body's immune system, is a feature of many diseases including infection, autoimmune disorders and benign and malignant hyperplasia. The identification of genetic factors which influence susceptibility to such disorders will provide important new insights into inflammatory disease, and may yield important new diagnostic and/or prognostic tests and treatments.

Psoriasis is a chronic inflammatory cutaneous disorder which affects approximately 2% of the population in the UK and US, and causes varying degrees of physical discomfort, pain and disability. Psoriasis manifests itself as red scaly skin patches, principally on the scalp, elbows and knees, and is caused by epidermal hyperproliferation, and abnormal differentiation and infiltration of inflammatory cells. Psoriasis may also be associated with other inflammatory diseases such as arthritis, Crohn's disease, and HIV infection. Population, family, and twin studies all suggest an important genetic component in the pathogenesis of psoriasis, coupled with environmental triggers such as streptococcal infection and stress.

Psoriasis is one of a number of autoimmune diseases that display significant human leukocyte antigen (HLA) associations. The analysis of population-specific HLA haplotypes has provided evidence that susceptibility to psoriasis is linked to the class I and II major histocompatibility complexes (MHC) on human chromosome 6. These studies show that psoriasis consists of two distinct disease subtypes (Type I and Type II), which differ in age of onset and in the frequency of HLA types. Type I psoriasis has an age of onset of prior to 40 years and HLA types Cw6, B57, and DR7 are strongly increased. Patients with Type I psoriasis are much more likely to have a positive family history for the disease. In contrast, only about 10% of Cw6-positive individuals develop Type II psoriasis disease, with HLA-Cw2 being over-represented in this group.

Linkage analysis and association studies suggest the presence of a major genetic determinant of psoriasis within the MHC, the strongest candidate gene marker being HLA-C. The most significant association has been shown between HLA-Cw6 and disease Type IA, which has the earliest onset of disease at 0 to 20 years. However, specific involvement of the HLA-Cw6 genotype in disease pathogenesis has yet to be established. At present, the causes of psoriasis are unknown. There is no specific test for psoriasis or susceptibility thereto, and diagnosis is based solely on clinical examination and skin histopathology.

The present invention aims to overcome or ameliorate previous limitations in the art by providing means and methods for the detection and treatment of individuals having, or being susceptible to inflammatory diseases such as psoriasis.

Thus, in a first aspect of the present invention, there is provided a method of diagnosing, or determining susceptibility of a subject to, inflammatory disease such as psoriasis, the method comprising determining the presence of one or more polymorphisms in the SEEK1 gene or protein. The method may be used to identify the presence of a combination of polymorphisms in a subject which define a haplotype linked to inflammatory disease. The haplotype may be any particular combination of the polymorphisms, optionally including known polymorphisms.

The present invention is based upon the realisation that SEEK1 is involved in epidermal differentiation, and the gene is involved in determining onset of inflammatory disease. SEEK1 is expressed in skin, in particular keratinocytes. The SEEK1 gene is located approximately 160 kb telomeric of the HLA-C locus, in a cluster of non-HLA genes. This gene cluster, termed the MHC epidermal gene cluster (MHC-EGC), spans approximately 50 kb genomic DNA and contains 5 genes, HCR, SPR1, CDSN, STG and SEEK1. SEEK1 is transcribed in the opposite orientation to the other four genes. The SEEK1 gene consists of 6 exons spanning approximately 24.8 kb of genomic DNA sequence. A SEEK1 mRNA transcript of 861 bp has been reported (Genbank accession AB031479) producing a predicted peptide 152 amino acids in length, which is rich in proline and serine residues, a characteristic feature of proteins involved in epidermal differentiation (South et al. (1999) J. Invest. Dermat. 112:910-918). EST sequences with homology to SEEK1 are reported to have been isolated from cDNA libraries synthesised from colon, uterus, ovary, testis and breast tissues (Genbank accession numbers A1343394, A1339603, AA127234, A1208110, A1379146, R50247, AA045454, A1243345, BE042193). Unlike HCR, SPR1, CDSN and STG, no mouse orthologue of the SEEK1 gene has been identified.

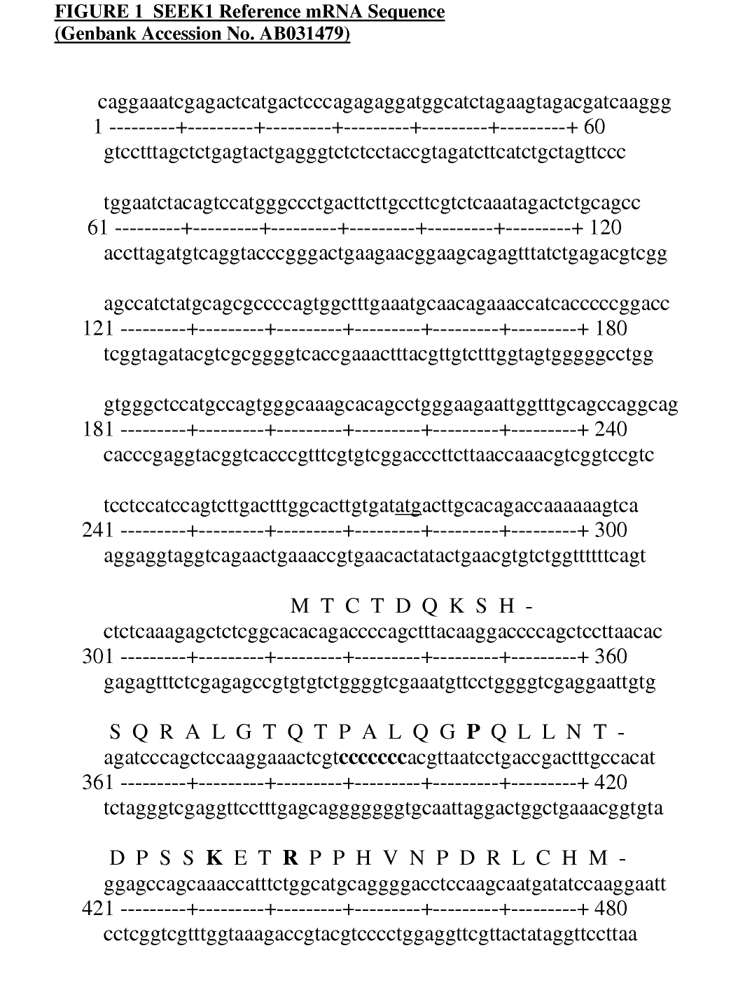

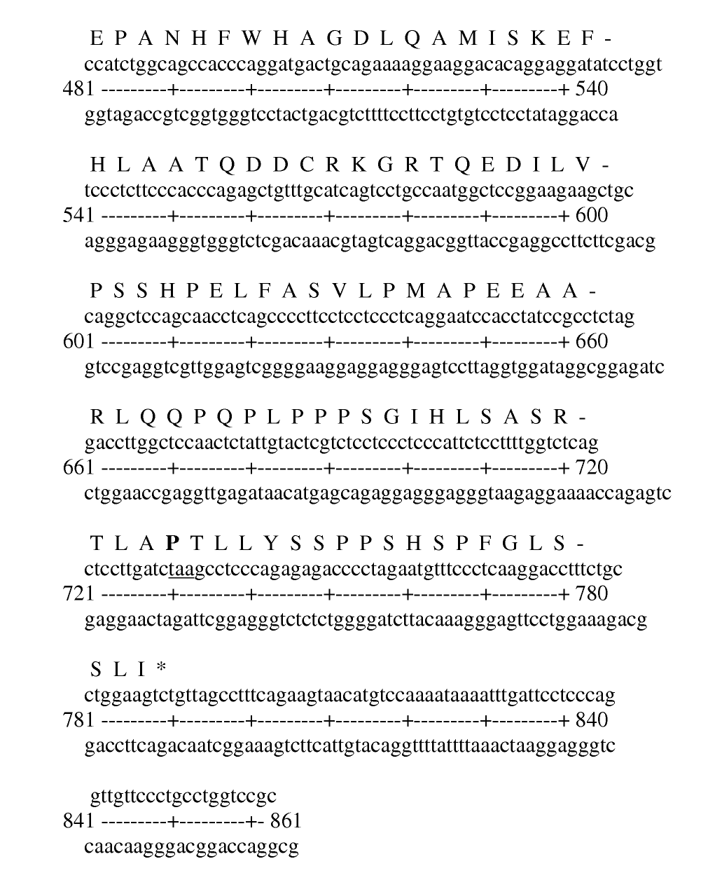

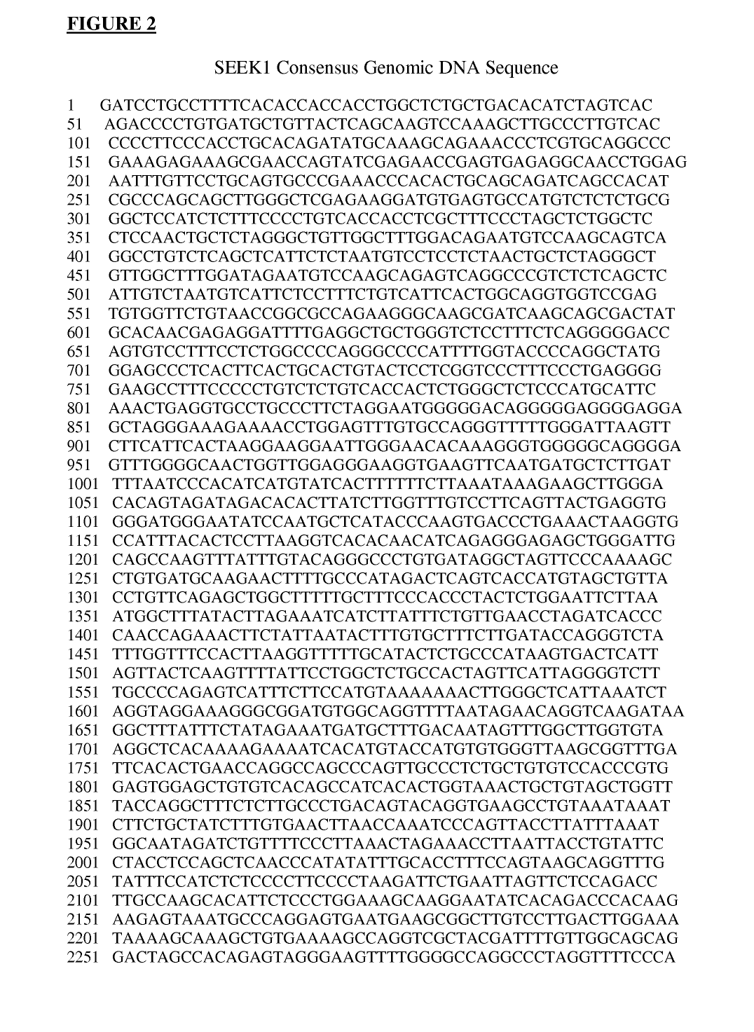

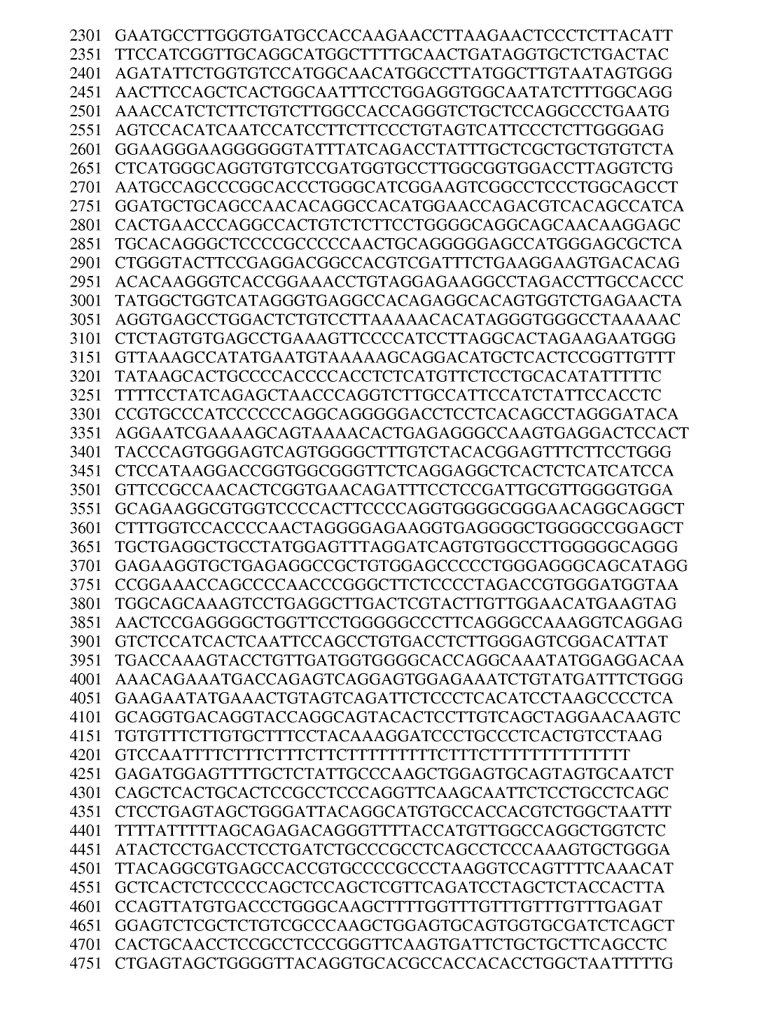

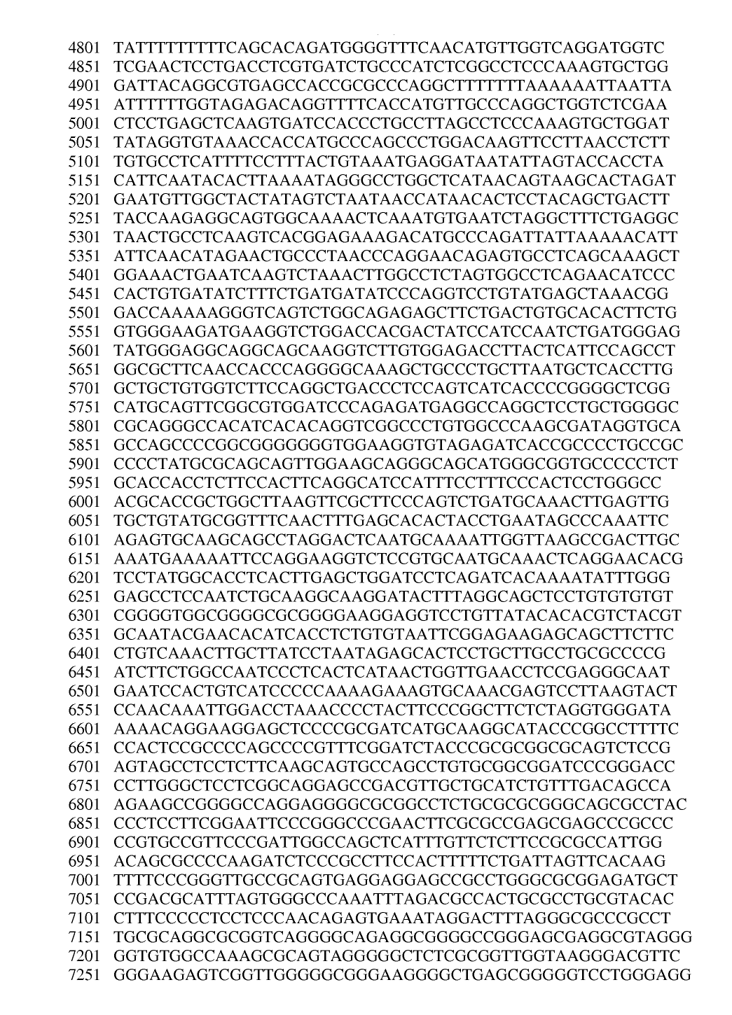

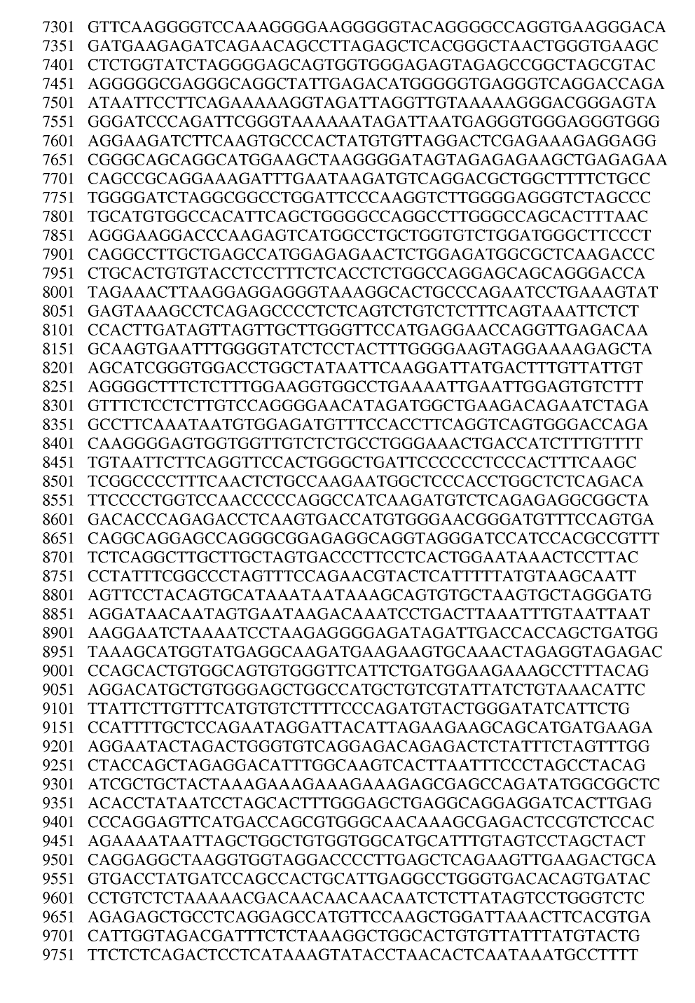

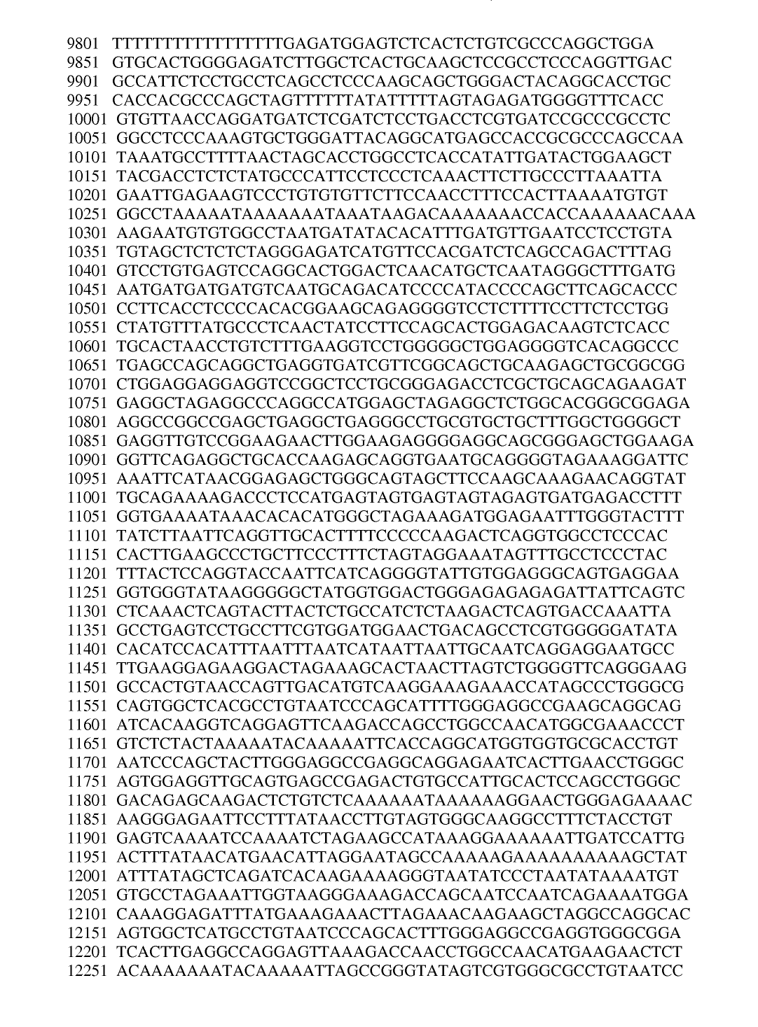

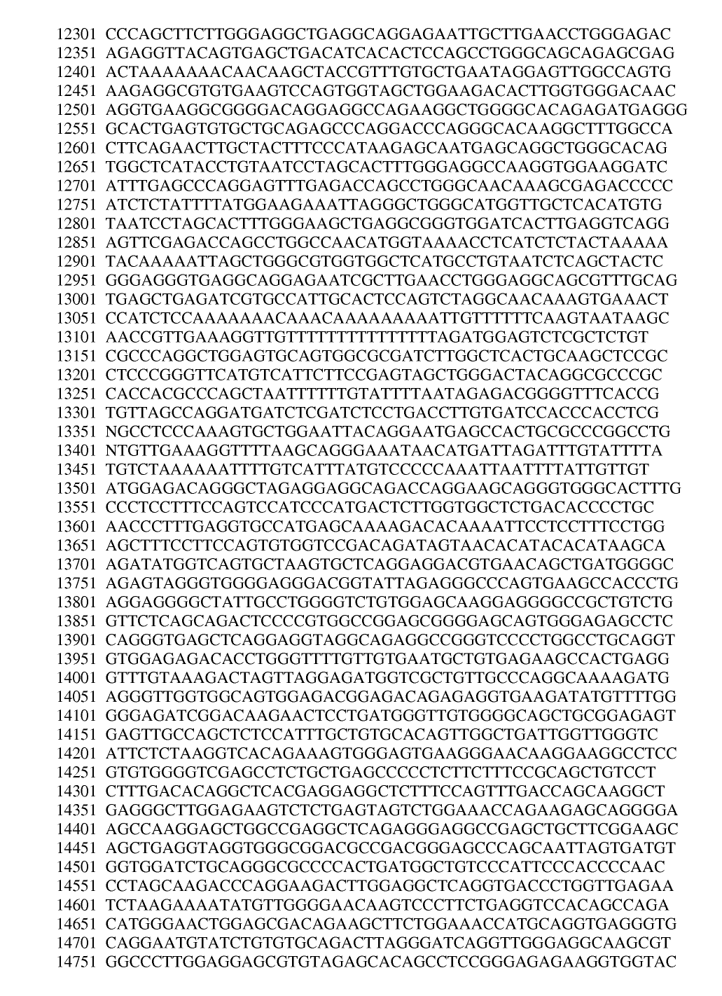

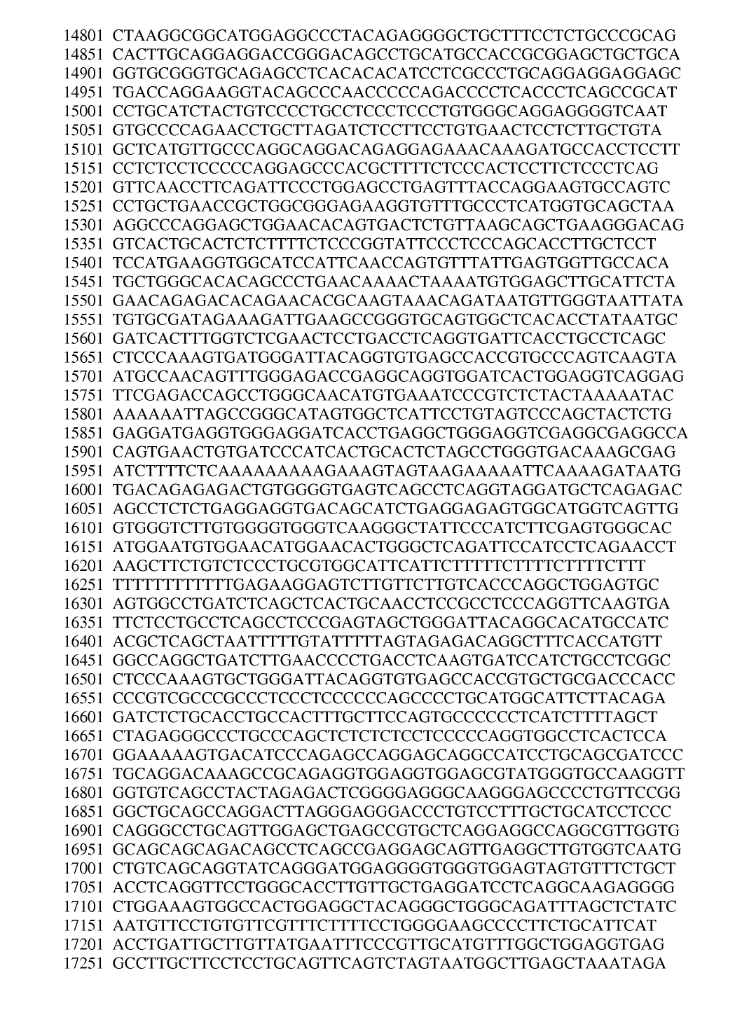

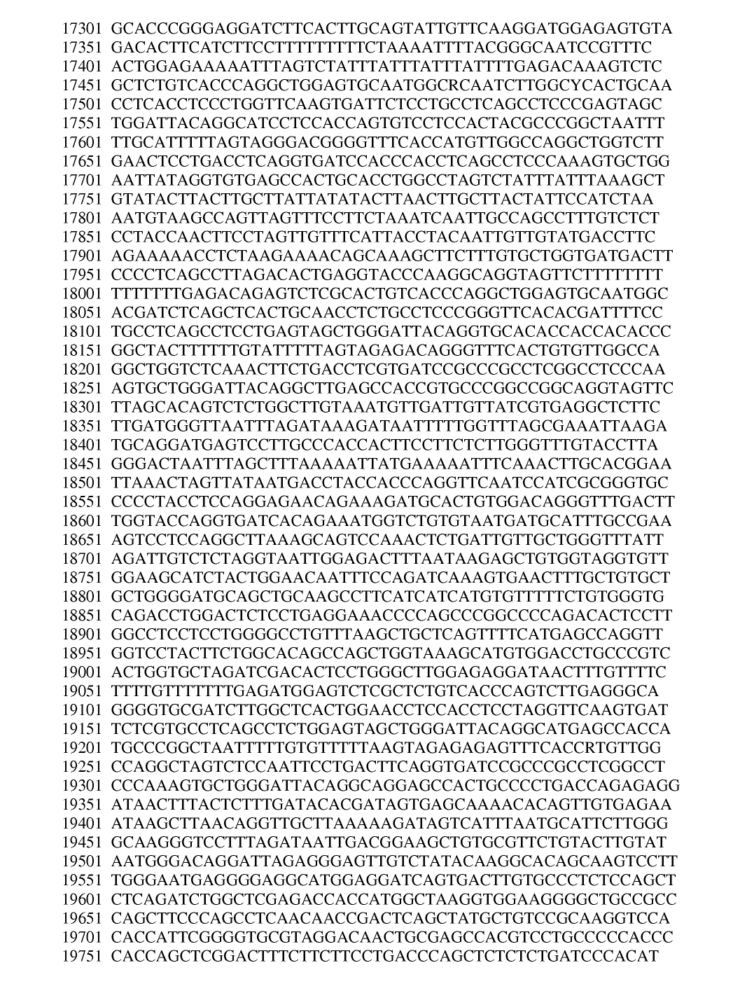

In the present text, and according to the present invention, the SEEK1 gene is that of GenBank Accession No. AP000510, which includes the 5′ promoter sequences, coding and non-coding exonic sequences, intronic sequences and 3′ untranslated sequences, all present on the MHC region of chromosome 6p21.3. The mRNA clone of SEEK1 (GenBank Accession No. AB031479) is shown in FIG. 1. A consensus genomic DNA sequence for SEEK1 is set out in FIG. 2.

A polymorphism is typically defined as two or more alternative sequences, or alleles, of a gene or protein in a population. A polymorphic site is the location at which divergence in sequence occurs. Examples of the ways in which polymorphisms are manifested include restriction fragment length polymorphisms, variable number of tandem repeats, hypervariable regions, minisatellites, di- or multi-nucleotide repeats, insertion elements and nucleotide or amino acid deletions, additions or substitutions. The first identified allele is usually referred to as the reference allele, or the wild type. Additional alleles are usually designated alternative or variant alleles. Herein, the sequence detailed in GenBank Accession No AP000510, the SEEK1 consensus genomic DNA sequence or FIG. 1 are designated the reference sequence. The Genbank sequence AP000510 and FIG. 1 are not part of the invention. Nucleic acid sequences which differ from the sequence of AP000510, the consensus sequence herein, or FIG. 1 at one or more positions may be referred to as variants.

A single nucleotide polymorphism is a variation in sequence between alleles at a site occupied by a single nucleotide residue. Single nucleotide polymorphisms (SNP's) arise from the substitution, deletion or insertion of a nucleotide residue at a polymorphic site. Typically, this results in the site of the variant sequence being occupied by any base other than the reference base. For example, where the reference sequence contains a “T” base at a polymorphic site, a variant may contain a “C”, “G” or “A” at that site. Single nucleotide polymorphisms may result in corresponding changes to the amino acid sequence. For example, substitution of a nucleotide residue may change the codon, resulting in an amino acid change. Similarly, the deletion or insertion of three consecutive bases in the nucleic acid sequence may result in the insertion or deletion of an amino acid residue. For ease of reference, where a single nucleotide polymorphism of the present invention results in the insertion or deletion of a nucleotide or amino acid residue, the numbering system of FIG. 1, the consensus sequence herein and AP000510 have been maintained.

The single nucleotide polymorphisms of the present invention which occur within the protein coding sequence may contribute to the phenotype of an organism by affecting protein structure or function. The effect may be neutral, beneficial or detrimental, depending upon the circumstances. Whatever the effect, the identification of such polymorphisms enables for the first time determination of susceptibility to disease, and new methods of treatment. The single nucleotide polymorphisms of the invention which occur in the non-coding 5′ or 3′ untranslated regions, may not affect protein sequence, but may exert phenotypic effects by RNA transcription, processing and/or translation. A polymorphism may affect more than one phenotypic trait or may be related to a specific phenotype. In the present invention, polymorphisms in the SEEK1 gene are likely to affect the phenotype of an individual with respect to SEEK1 mediated disease, such as inflammatory disease, in particular psoriasis.

In a preferred embodiment, the present invention provides a method of diagnosing or determining susceptibility to SEEK1 mediated disease, said method comprising determining the presence of a nucleotide substitution, deletion or insertion at one or more of positions 51814, 51789, 51759, 51570, 51505, 51462, 51265, 51216, 51124, 51078, 51017, 51008, 50920, 50901, 50801, 50049, 49405-49407, 49160, 49133, 49045, 49038, 49017, 48920, 48773, 47938, 47868, 47852, 47826, 47661, 47645, 47567, 47547, 47508, 47507, 47438, 46831, 46806, 46784, 39881, 39880, 39851, 39725, 39722, 39702, 35884, 35732, 27006, 26915, 26770, 26724, 26694 26684, 26675-26682, 26576, 26539, 25534, 25458 and 25449 of SEEK1 gene as represented by FIG. 2. Some of these positions correspond to positions: 16549, 16548, 16519, 16393, 16390, 16370, 12553, 12401, 3676, 3585, 3444, 3394, 3364, 3354, 3352, 3247, 3210, 2205, 2126, and 2120 of Genbank sequence AP000510. In the present invention, it is the position of the polymorphism which is the novel and limiting feature: the reference to the gene sequence simply confirms that the polymorphism is present in the SEEK1 gene or protein. The sequence need or may not be fully identical to that given in any one of the reference sequences. This applies both to gene and protein reference sequences. The relationship between the positions in the consensus sequence of FIG. 2 and in the Genbank sequence AP000510 are as follows:

| SNP name, referring | SNP Number, referring |
|---|---|
| to Genbank | to consensus sequence |
| sequence AP000510 | of FIG. 2 |
| G16549A | SNP39 |
| G16548A | SNP40 |
| C16519T | SNP41 |
| A16393G | SNP42 |
| G16390A | SNP43 |
| G16370A | SNP44 |
| C12553T | SNP45 |
| G12401T | SNP46 |
| T3676C | SNP47 |
| A3585G | SNP48 |
| A3444G | SNP49 |
| C3394A | SNP50 |
| G3364A | SNP51 |
| G3354A | SNP52 |
| C3352Ins/Del[C] | SNP53 |
| C3352Ins/Del[C] | SNP53 |
| C3352Ins/Del[C] | SNP53 |
| A3247T | SNP54 |
| G3210A | SNP55 |
| C2205T | SNP56 |
| C2126Del[C] | SNP57 |
| C2120T | SNP58 |

These novel polymorphisms in the SEEK1 gene, at the positions indicated above, have been identified as being involved in SEEK1 mediated disease. In particular, the polymorphisms of the present invention may be useful in identifying individuals being susceptible or resistant to SEEK1-mediated disease, and in the diagnosis or treatment of such conditions.

In this text, diseases in which SEEK1 is implicated in the pathology will be referred to as “SEEK1-mediated disease”. Such diseases include inflammatory disease such as psoriasis. In particular, the inflammatory disease is of the skin, most particularly skin psoriasis.

The single polymorphisms of the invention have each been given a positional reference with respect to the consensus sequence of FIG. 2 (see Table 1(iii)). Some of them also have a positional reference with respect to sequence of GenBank Accession No. AP000510 (see Table 1 (i), column 1). However, it should be noted that the native SEEK1 gene is transcribed in the opposite orientation to AP000510 and the consensus sequence. In addition, for ease of reference, polymorphisms occurring in the coding sequence of SEEK1 are also given a positional reference with respect to the SEEK1 mRNA sequence. The fragments of the SEEK1 gene comprising the polymorphisms (as shown in Table 1(i), column 3 and in Table 1 (iii), column 3) are fragments of the sequence of GenBank Accession No AP000510. These fragments can be readily aligned with the genomic sequence of GenBank Accession No. AP000510, or other clones of this region, using methods known to the person skilled in the art, for example by comparing the nucleotide sequence of the fragment with the sequence of the MHC-EGC region by using computer programs such as DNASIS (Hitachi Engineering, Inc.) or Word Search or FASTA of the Genetic Computer Group (Madison, Wis.).

The present invention can also thus refer to the polymorphisms in the SEEK1 gene (coding and non-coding) and the SEEK1 protein (coding) by reference to the fragments as set out in Table 1(i) and Table 1(iii).

Any method, including those known to persons skilled in the art, may be used to determine which allele of one or more polymorphisms is present. Preferably, the method comprises first removing a sample from a subject. More preferably, the method comprises isolating from a sample a polynucleotide or protein to determine therein which allele of one or more polymorphisms of the invention is present. Any biological sample comprising cells containing nucleic acid or protein is suitable for this purpose. Examples of suitable samples include whole blood, semen, saliva, tears, buccal, skin or hair. For analysis of cDNA, mRNA or protein, the sample must come from a tissue in which the SEEK1 gene is expressed, and thus it is preferable to use skin samples.

Any method for determining alleles in a polynucleotide may be used, including those known to persons skilled in the art. One example of a widely-available technique is direct DNA sequencing of PCR products containing the polymorphism to be tested. However, and preferably, the method may comprise the use of anti-sense polynucleotides, such as those of the present invention, as defined below. Such polynucleotides may include sequences which are able to distinguish between alleles of one or more polymorphisms, by preferential binding, and sequences which hybridise under stringent conditions to a region either side of a polymorphism to enable amplification of one or more of the polymorphisms.

Methods of this embodiment include those known to persons skilled in the art, for example, direct probing, allele specific hybridisation, and PCR-based methods including sequencing of PCR products, Allele Specific Amplification (ASA), RFLP, single base extension and rolling circle amplification following allele-specific ligation.

Determination of an allele of a polymorphism using direct probing involves the use of anti-sense sequences. These may be prepared synthetically or by nick translation. The anti-sense probes may be suitably labelled using, for example, a radiolabel, enzyme label, fluoro-label, biotin-avidin label for subsequent visualization in, for example, a southern blot procedure. A labelled probe may be reacted with a sample DNA or RNA, and the areas of the DNA or RNA which carry complimentary sequences will hybridise to the probe, and become labelled themselves. The labelled areas may then be visualized, for example by autoradiography.

Preferably, the method may first comprise the amplification of a region of the SEEK1 gene containing one or more of the polymorphic sites of the invention, for example, using PCR techniques. Probes of the present invention may be useful for this purpose.

The above described methods may require amplification of the DNA sample from the subject, and this can be done by techniques known in the art, such as PCR (see PCR Technology: Principles and Applications for DNA Amplification (ed. H. A. Erlich, Freeman Press, NY 1992; PCR Protocols. A Guide to methods and Applications (eds. Innis et al., Academic press, San Diego, Calif. 1990); Mattila et al., Nucleic Acids Res. 19 4967 (1991); Eckert et al., PCR Methods and Applications 117 (1991) and U.S. Pat. No. 4,683,202. Other suitable amplification methods include ligase chain reaction (LCR) (Wu et al., Genomics 4 560 (1989); Landegran et al., Science 241 1077 (1988)), transcription amplification (Kwoh et al., Proc Natl Acad Sci USA 86 1173 (1989)), self sustained sequence replication (Guatelli et al., Proc Natl Acad Sci USA 87 1874 (1990)) and nucleic acid based sequence amplification (NASBA). The latter two methods both involve isothermal reactions based on isothermal transcription which produce both single stranded RNA and double stranded DNA as the amplification products, in a ratio of 30 or 100 to 1, respectively.

It may often be desirable to identify the presence of multiple single nucleotide polymorphisms in a sample from a subject. This may be the case in the present invention where the SEEK1 gene contains at least 21 polymorphisms, each of which may be indicative of a different phenotype of inflammatory disease. For this purpose, nucleic acid arrays may be useful, as described in WO95/11995. The array may contain a number of probes, each designed to identify one or more of the above single nucleotide polymorphisms of the SEEK1 gene, as described in WO95/11995.

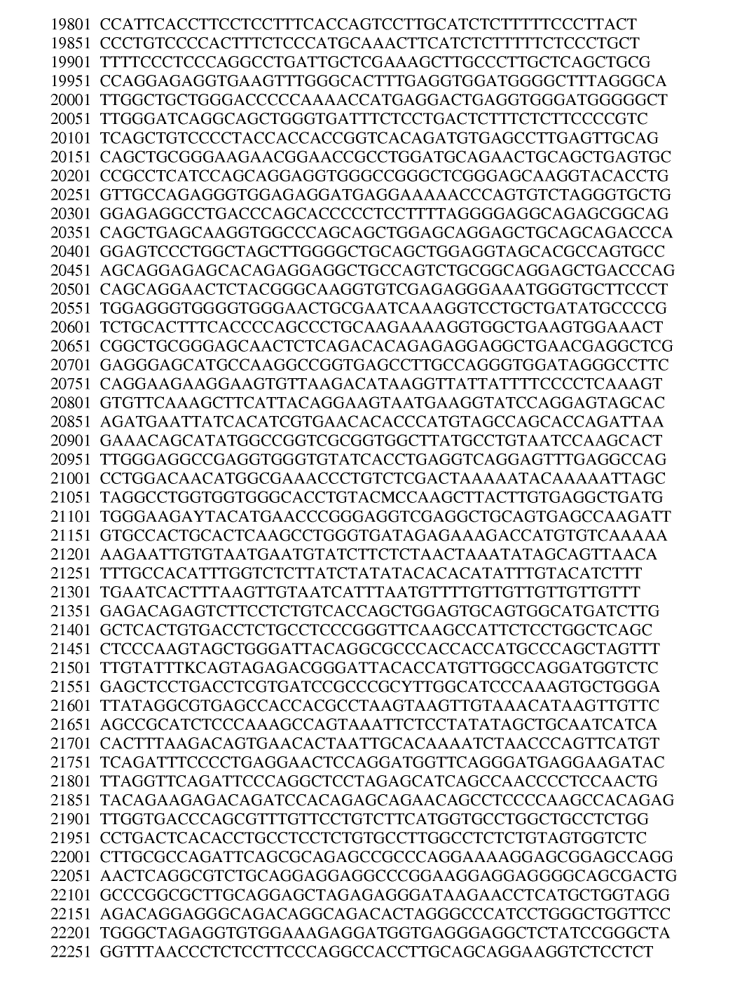

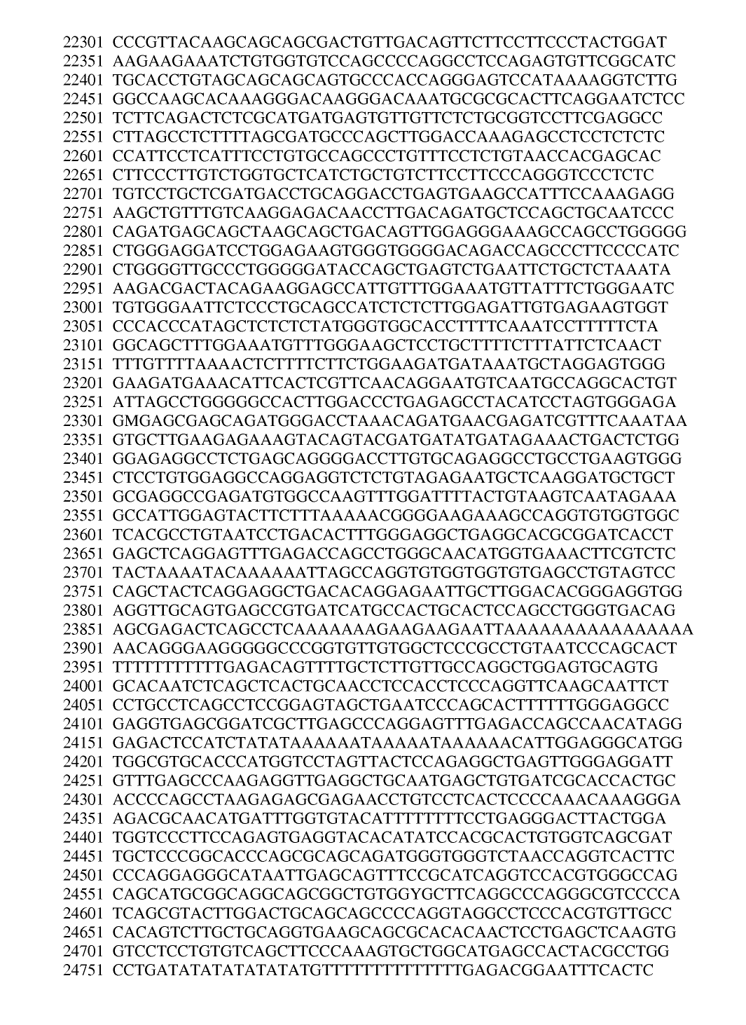

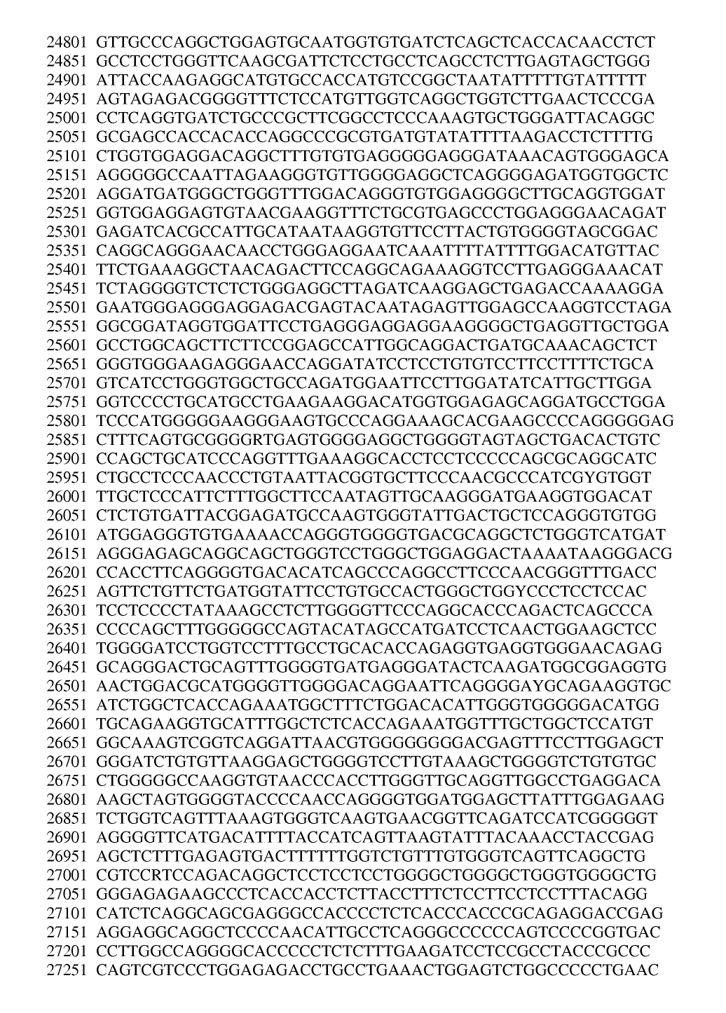

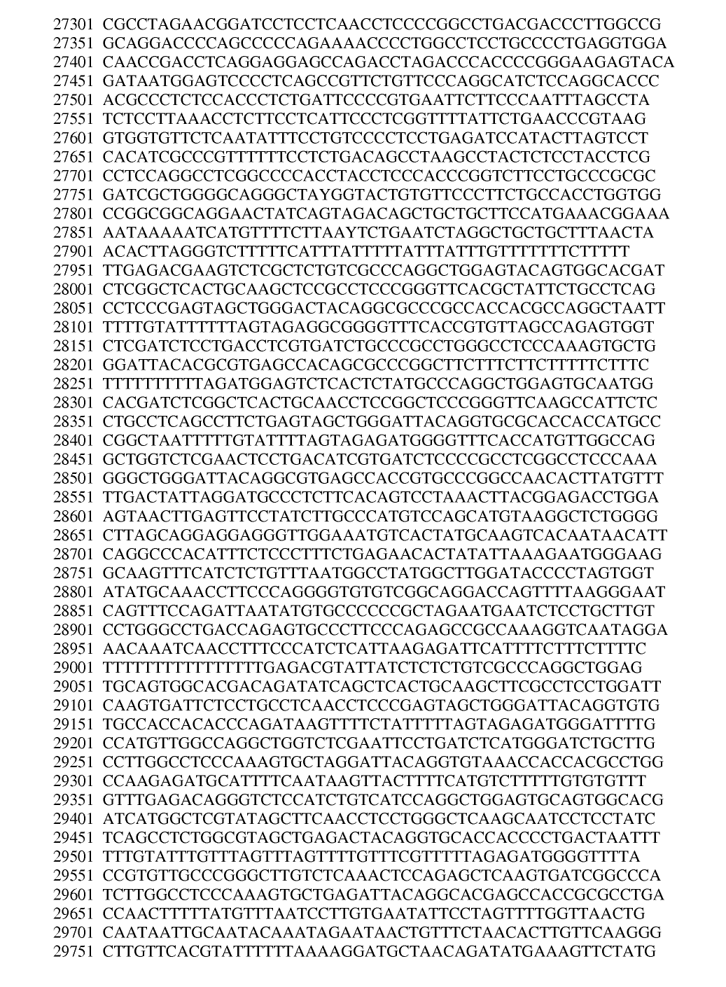

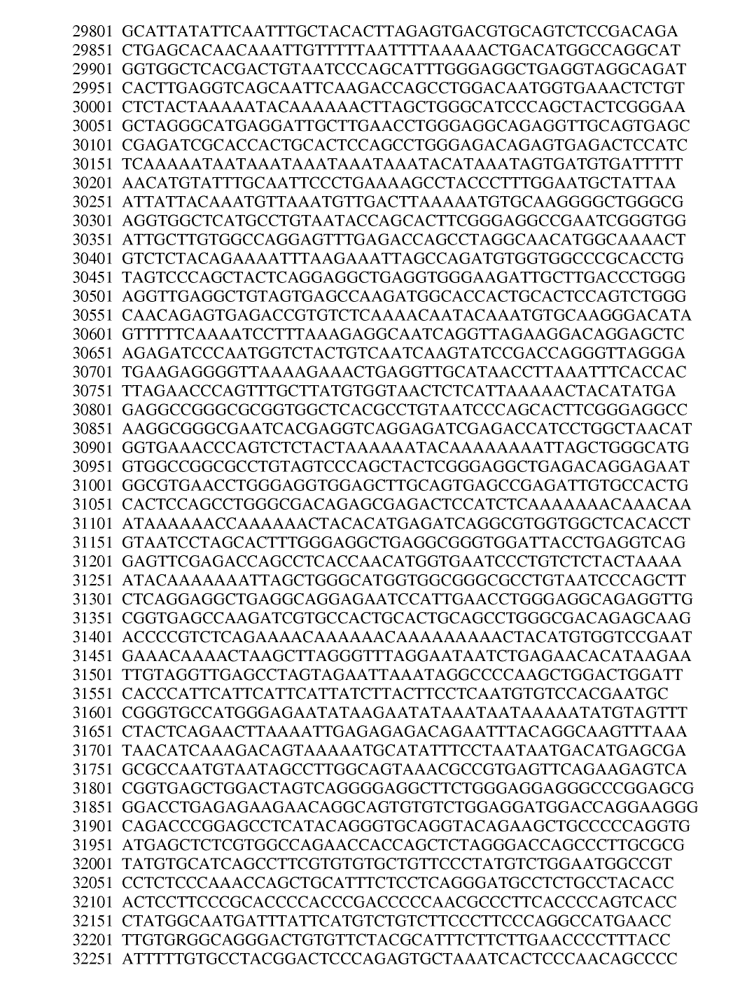

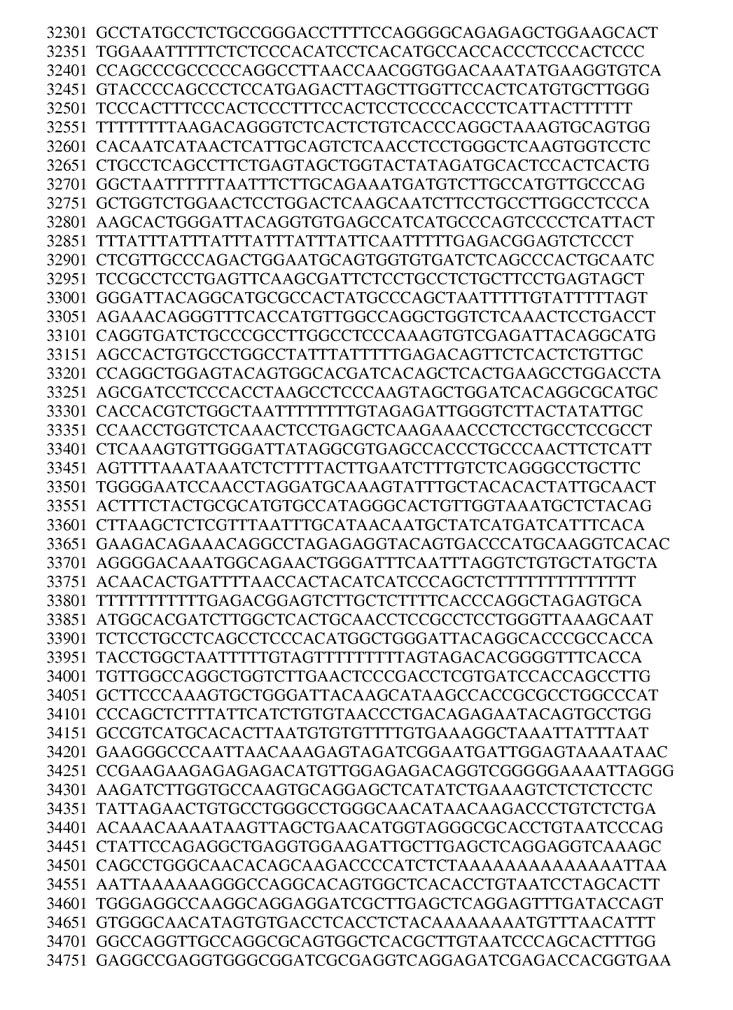

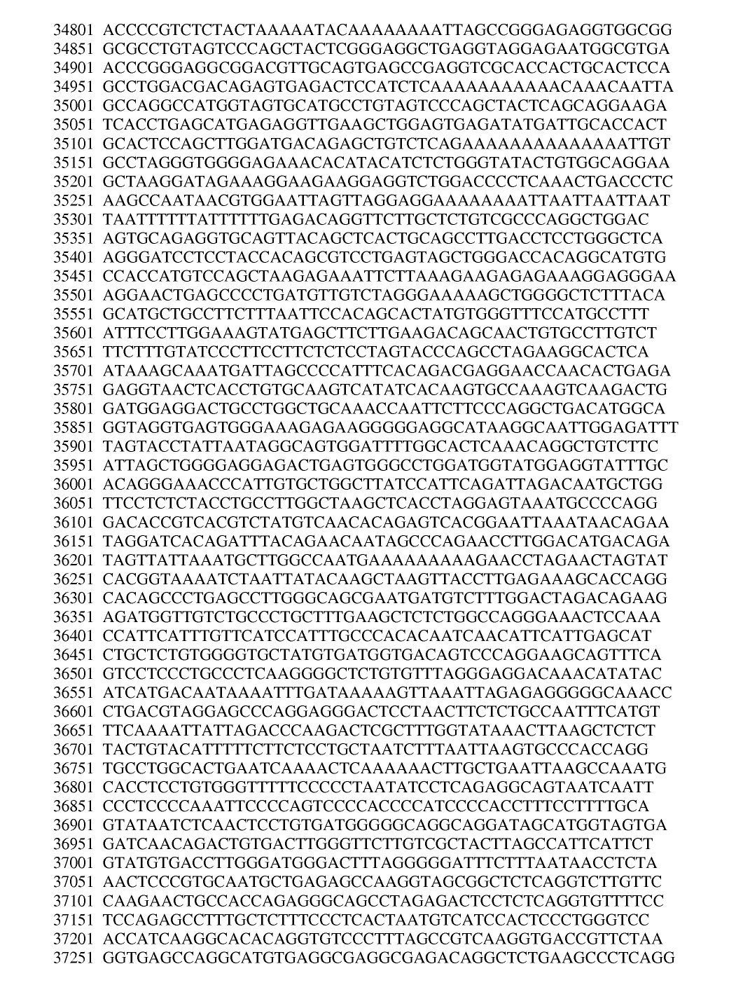

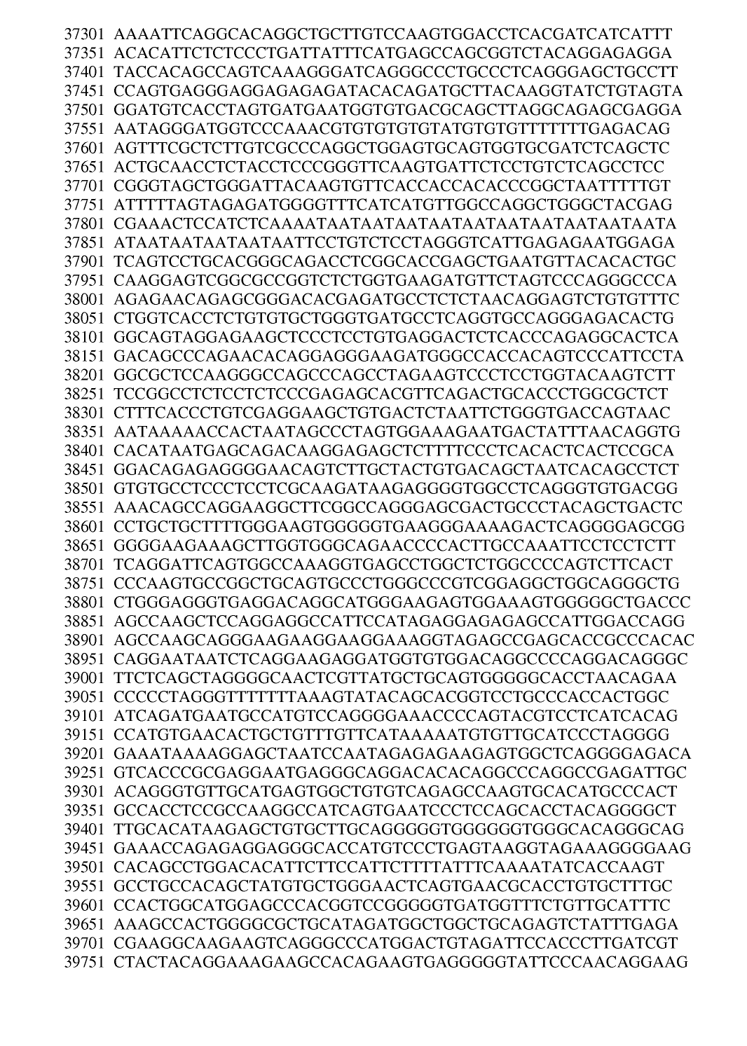

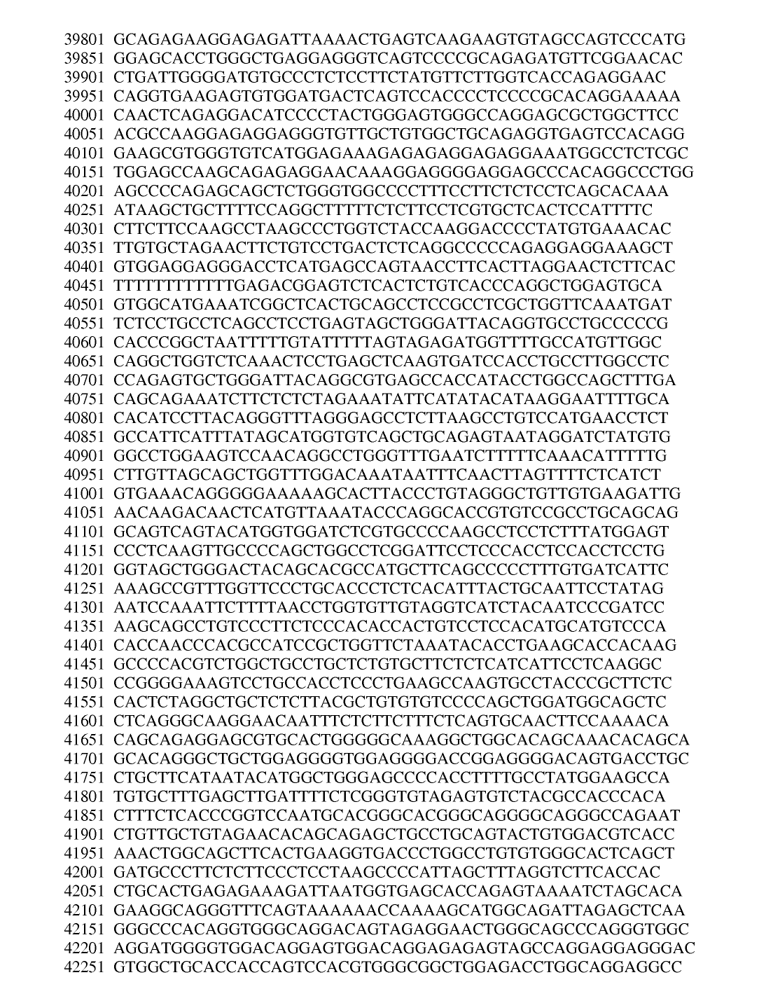

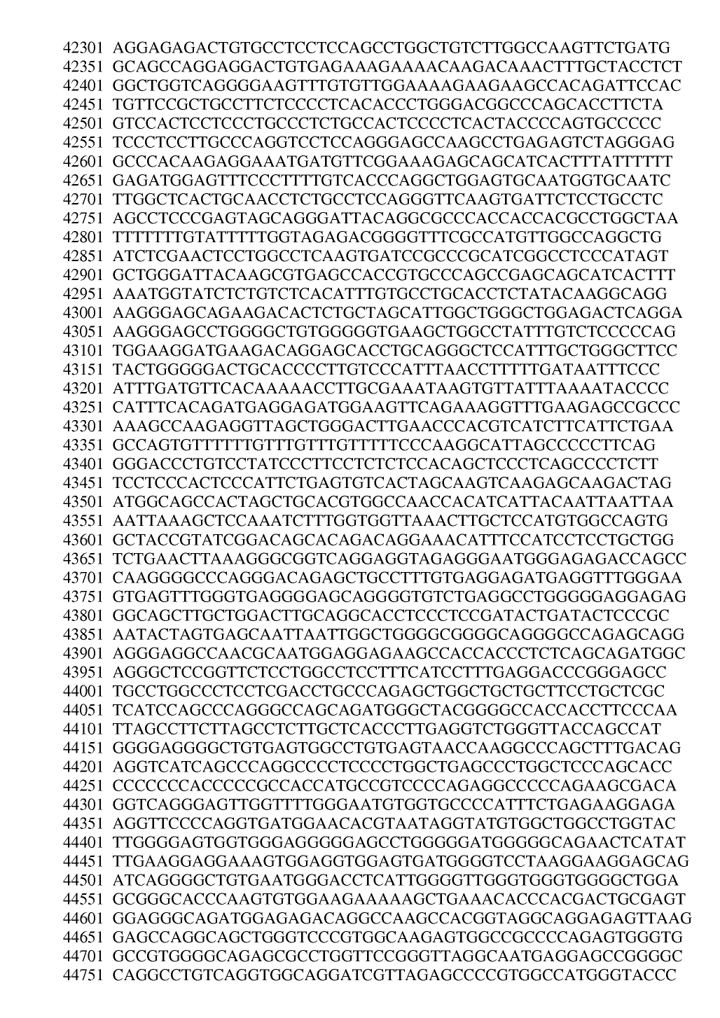

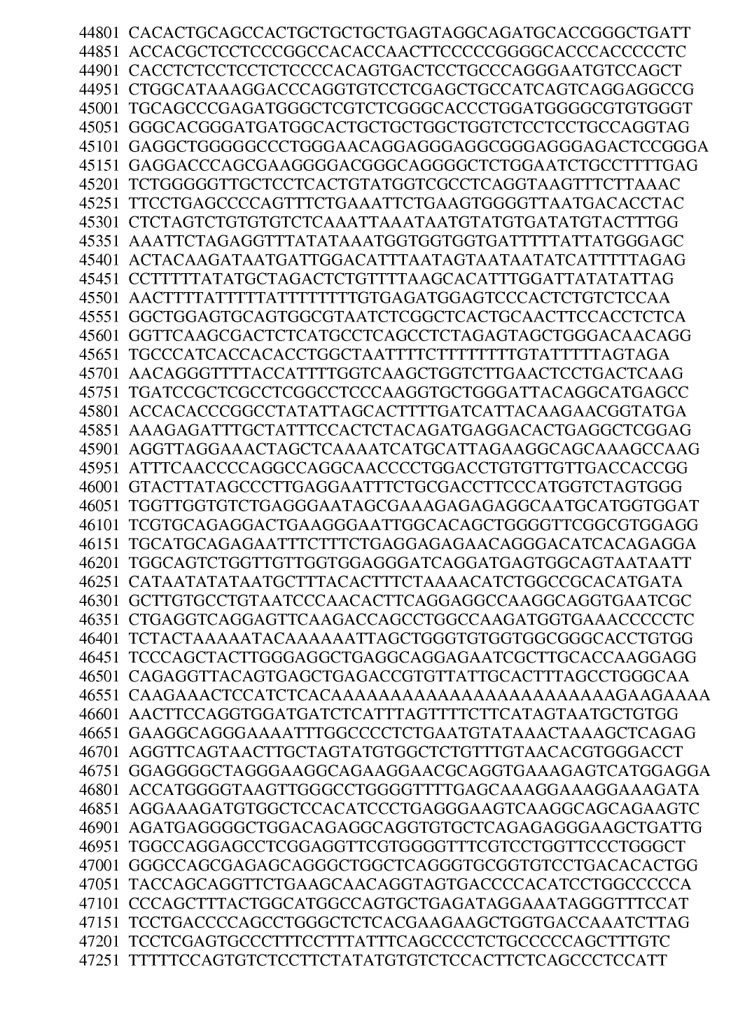

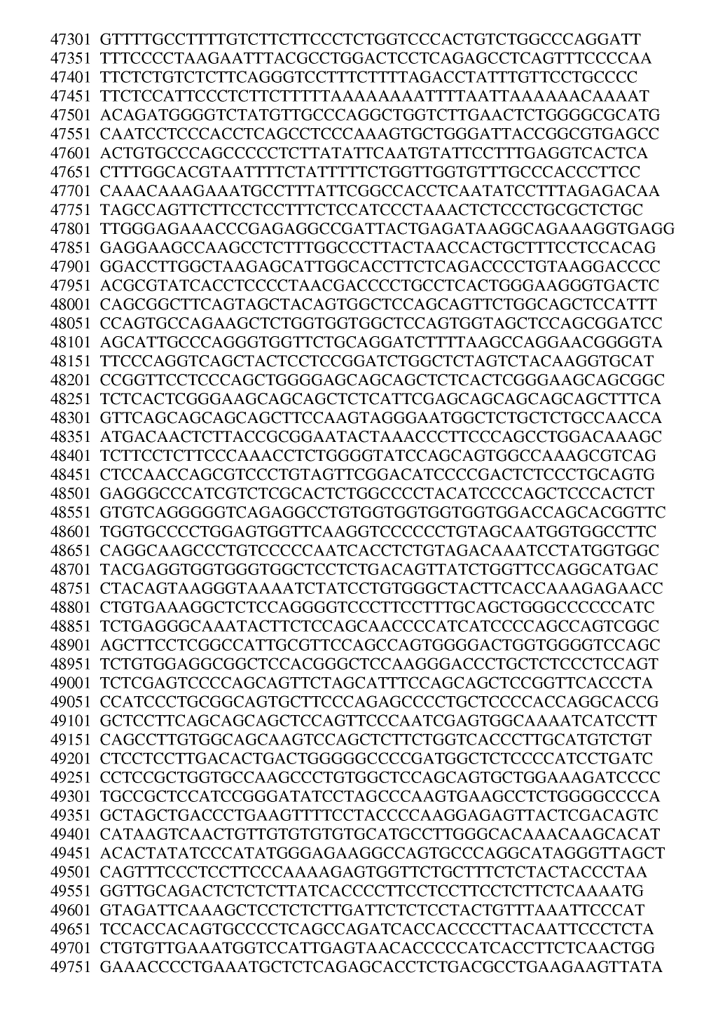

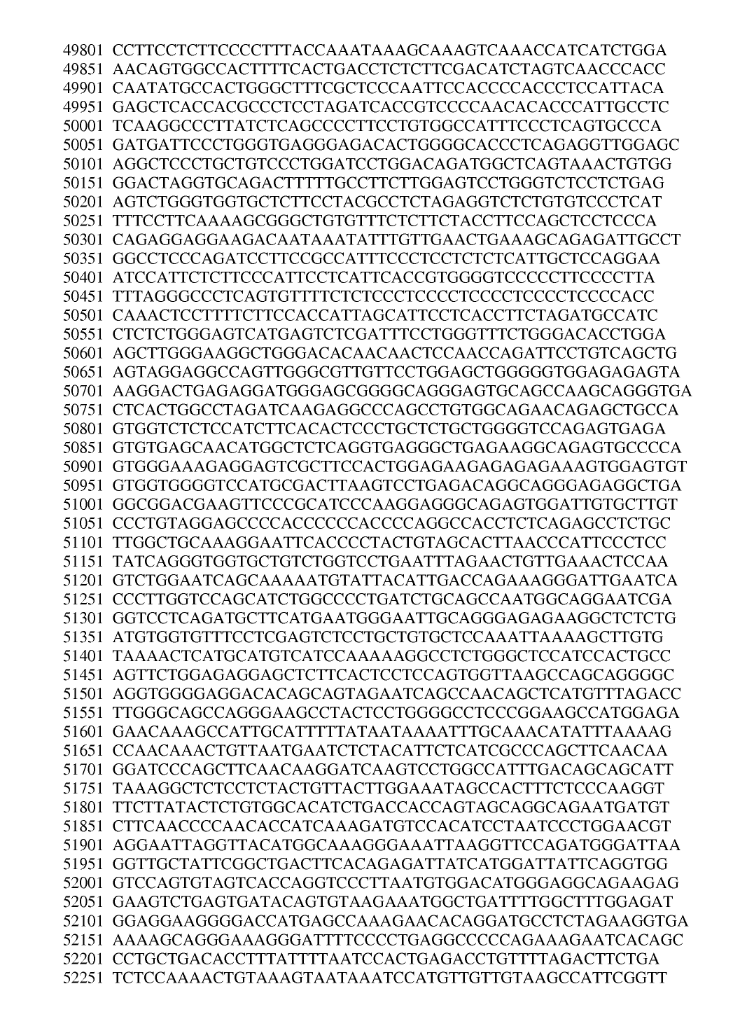

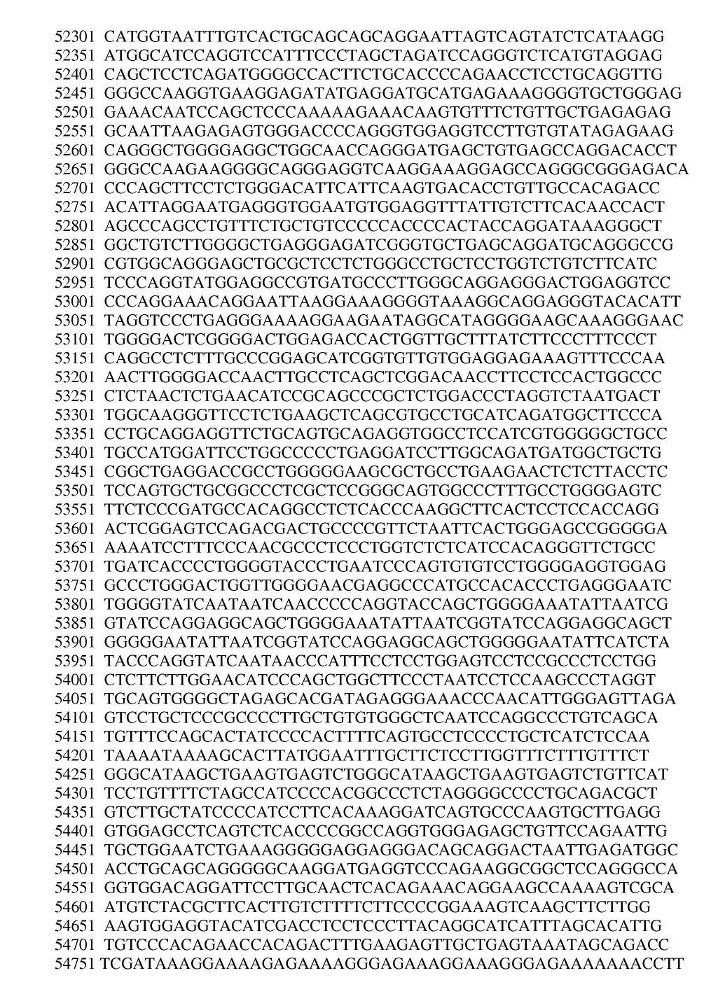

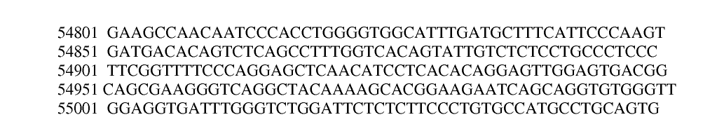

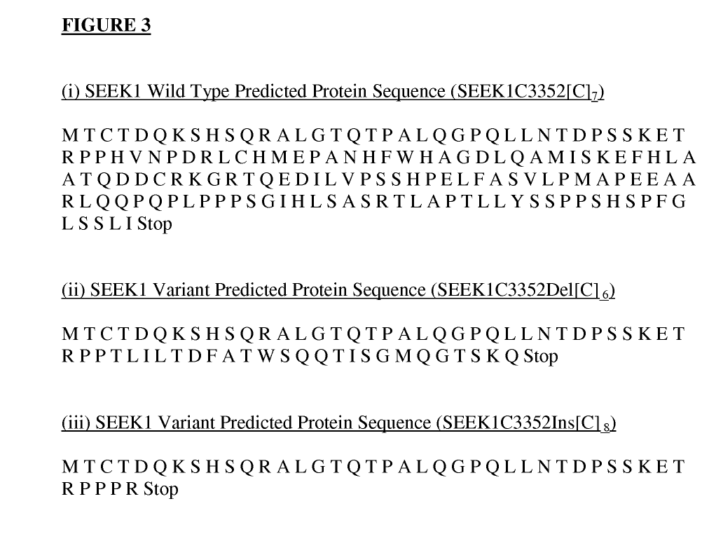

In a preferred embodiment of the first aspect, there is provided a method for diagnosing or determining susceptibility to SEEK1 mediated disease, the method comprising determining the presence of an amino acid substitution, deletion or insertion at one or more of positions 24, 34, 37, 40 or 133 of the SEEK1 amino acid sequence, represented by FIG. 3(i), or the presence of a protein fragment having the amino acid sequence as represented by FIG. 3(ii) or 3 (iii). Any method for determining the presence of a particular form, or allele, of a protein is present, may be used. One such method involves the use of antibodies in diagnosing or determining susceptibility to SEEK1 mediated disease. The method may comprise removing a sample from a subject, contacting the sample with an antibody to an antigen of a SEEK1 protein or protein fragment and detecting binding of the antibody to the antigen, wherein binding is indicative of the presence of a particular allele or form of the protein and thus risk to SEEK1 mediated disease. Tissue samples as described above are suitable for this method.

The detection of binding of the antibody to the antigen in a sample may be assisted by methods known in the art, such as the use of a secondary antibody which binds to the first antibody, or a ligand. Immunoassays including immunofluorescence assays (IFA) and enzyme linked immunosorbent assays (ELISA) and immunoblotting may be used to detect the presence of the antigen. For example, where ELISA is used, the method may comprise binding the antibody to a substrate, contacting the bound antibody with the sample containing the antigen, contacting the above with a second antibody bound to a detectable moiety (typically an enzyme such as horse radish peroxidase or alkaline phosphatase), contacting the above with a substrate for the enzyme, and finally observing the colour change which is indicative of the presence of the antigen in the sample.

In a second aspect of the present invention, there is provided an isolated or recombinant polynucleotide comprising a nucleic acid sequence encoding the SEEK1 gene as represented by the consensus sequence of FIG. 2, of AP000510, wherein the nucleic acid sequence comprises a nucleotide substitution, deletion or insertion at one or more of positions 51814, 51789, 51759, 51570, 51505, 51462, 51265, 51216, 51124, 51078, 51017, 51008, 50920, 50901, 50801, 50049, 49405-49407, 49160, 49133, 49045, 49038, 49017, 48920, 48773, 47938, 47868, 47852, 47826, 47661, 47645, 47567, 47547, 47508, 47507, 47438, 46831, 46806, 46784, 39881, 39880, 39851, 39725, 39722, 39702, 35884, 35732, 27006, 26915, 26770, 26724, 26694 26684, 26675-26682, 26576, 26539, 25534, 25458 and 25449 of FIG. 2.

The polynucleotide of this invention is preferably DNA, or may be RNA or other options.

As discussed above, where a single nucleotide polymorphism of the present invention comprises a nucleotide substitution, the substitution may comprise the replacement of the reference base at a polymorphic site with any other base. Each nucleic acid sequence of Table 1(i), column 3 and Table 1(iii), column 3 comprising a single nucleotide polymorphism represents a preferred embodiment of the invention.

It will be appreciated by those skilled in the art that SEEK1 gene sequences of the invention may comprise one or more nucleotide substitutions, deletions or insertions in addition to one or more of the single nucleotide polymorphisms of the invention.

In a third aspect, fragments of the above polynucleotides are provided, which comprise one or more nucleotide substitutions, insertions or deletions at one or more of the above mentioned positions of the SEEK1 gene, as represented by consensus sequence. Preferably, a fragment may comprise, or even consist of, the polynucleotide sequence of Table 1 (i), column 3 or Table (iii), column 3. The novelty of a fragment according to the present embodiment may be easily ascertained by comparing the nucleotide sequence of a fragment with sequences catalogued in databases such as GenBank, or by using computer programs such as DNASIS (Hitachi Engineering, Inc.) or Word Search or FASTA of the Genetic Computer Group (Madison, Wis.).

Preferably, the fragments do not encode a full length protein, as is generally the case with the aforementioned polynucleotides of the second aspect, but otherwise satisfy the requirements of the second aspect. Preferred fragments may be 10 to 150 nucleotides in length. More preferably, the fragments are between 5 to 10, 5 to 20, 10 to 20, 20 to 50, or 50 to 100 nucleotides in length. For example, the fragments may be 5, 8, 10, 12, 15, 18, 20, 22, 25, 28, 30, or 35 nucleotides in length. The fragments may be useful in a variety of diagnostic, prognostic or therapeutic methods, or may be useful as research tools for example in drug screening.

In a fourth aspect of the invention, there is provided non-coding, complementary sequences which hybridise to the SEEK1 gene sequence. Such “anti-sense” sequences are useful as probes or primers for detecting an allele of a polymorphism of the invention, for example in the methods of the first aspect or in the regulation of the SEEK1 gene. They may also be used as agents for use in the identification and/or treatment of individuals having or being susceptible to SEEK1 mediated disease.

The anti-sense sequences of the invention include those which hybridise to an allele of a polymorphism of the invention, and also those which hybridise a region flanking the polymorphic site to enable amplification of an allele of one or more polymorphisms. Preferred anti-sense sequences are the complements of the sequences shown in Table 1(i) column 3 or Table 1(iii), column 3, or more preferably the complement of the sequence upstream and downstream of, and including, the polymorphism the anti-sense sequences may be useful as probes or primers. To be useful as a probe, the anti-sense sequence should bind preferentially one allele of one or more polymorphisms of the present invention and will, preferably, comprise the exact complement of one allele of one or more polymorphisms of the invention. Thus, for example, where the variant comprises a “G” residue at position 16549 of AP000510 (the same as SNP39 of the consensus sequence of FIG. 2), it is preferred that the anti-sense sequence will comprise a “C” residue. Such anti-sense sequences which are capable of specific hybridisation to detect a single base mis-match may be designed according to methods known in the art and described in Maniatis et al., Molecular Cloning: A Laboratory Manual 2^{nd} Edition (1989), Cold Spring Harbor, N.Y. and Berger et al., Methods in Enzymology 152: Guide to Molecular Cloning Techniques (1987) Academic Press Inc. San Diego, Calif.; Gibbs et al., Nuc Acids Res., 17: 2437 (1989); Kwok et al., Nucl Acids Res 18: 999; and Miyada et al., Methods Enzymol. 154: 94 (1987). Variation in the sequence of these anti-sense sequence is acceptable for the purposes of the present invention, provided that the ability of the anti-sense sequence to distinguish between alleles of a polymorphism is not compromised. Similarly, variation in the sequence of a primer sequence is acceptable, provided its ability to mediate amplification of a polymorphic site is not compromised. Preferably, a primer sequence will hybridise to the SEEK1 gene under stringent conditions which are defined below.

In relation to the present invention, “stringent conditions” refers to the washing conditions used in a hybridisation protocol. In general, the washing conditions should be a combination of temperature and salt concentration so that the denaturation temperature is approximately 5 to 20° C. below the calculated Tₘ of the nucleic acid under study. The Tₘ of a nucleic acid probe of 20 bases or less is calculated under standard conditions (1M NaCl) as [4° C.×(G+C)+2° C.×(A+T)], according to Wallace rules for short oligonucleotides. For longer DNA fragments, the nearest neighbour method, which combines solid thermodynamics and experimental data may be used, according to the principles set out in Breslauer et al., PNAS 83: 3746-3750 (1986). The optimum salt and temperature conditions for hybridisation may be readily determined in preliminary experiments in which DNA samples immobilised on filters are hybridised to the probe of interest and then washed under conditions of different stringencies. While the conditions for PCR may differ from the standard conditions, the Tₘ may be used as a guide for the expected relative stability of the primers. For short primers of approximately 14 nucleotides, low annealing temperatures of around 44° C. to 50° C. are used. The temperature may be higher depending upon the base composition of the primer sequence used.

The anti-sense polynucleotides of this embodiment may be the full length of the SEEK1 gene as represented by AP000510 or the consensus sequence of FIG. 2, or more preferably may be 5 to 200 nucleotides in length. Preferred polynucleotides are 5 to 10, 10 to 20, 20 to 50, 50 to 100 or 100 to 200 nucleotides in length. Primers, in particular, are typically 10 to 15 nucleotides long, and may occasionally be 16 to 25.

In a preferred embodiment, the polynucleotides of the aforementioned aspects of the invention may be in the form of a vector, to enable the in vitro or in vivo expression of the polynucleotide sequence. The polynucleotides may be operably linked to one or more regulatory elements including a promoter; regions upstream or downstream of a promoter such as enhancers which regulate the activity of the promoter; an origin of replication; appropriate restriction sites to enable cloning of inserts adjacent to the polynucleotide sequence; markers, for example antibiotic resistance genes; ribosome binding sites: RNA splice sites and transcription termination regions; polymerisation sites; or any other element which may facilitate the cloning and/or expression of the polynucleotide sequence. Where two or more polynucleotides of the invention are introduced into the same vector, each may be controlled by its own regulatory sequences, or all sequences may be controlled by the same regulatory sequences. In the same manner, each sequence may comprise a 3′ polyadenylation site. The vectors may be introduced into microbial, yeast or animal DNA, either chromosomal or mitochondrial, or may exist independently as plasmids. Examples of suitable vectors will be known to persons skilled in the art and include pBluescript II, LambdaZap, and pCMV-Script (Stratagene Cloning Systems, La Jolla (USA))

Appropriate regulatory elements, in particular, promoters will usually depend upon the host cell into which the expression vector is to be inserted. Where microbial host cells are used, promoters such as the lactose promoter system, tryptophan (Trp) promoter system, β-lactamase promoter system or phage lambda promoter system are suitable. Where yeast cells are used, preferred promoters include alcohol dehydrogenase I or glycolytic promoters. In mammalian host cells, preferred promoters are those derived from immunoglobulin genes, SV40, Adenovirus, Bovine Papilloma virus etc. Suitable promoters for use in various host cells would be readily apparent to a person skilled in the art (See, for example, Current Protocols in Molecular Biology Edited by Ausubel et al, published by Wiley).

In a fifth aspect of the present invention there is provided a protein or protein fragment comprising an amino acid substitution, deletion or insertion at one or more of positions 24, 34, 37, 40 or 133 of the amino acid SEEK1 sequence as represented by FIG. 3(i), or a SEEK1 protein fragment having the amino acid sequence represented by FIG. 3 (ii) or 3 (iii). Preferably, the protein or protein fragment is encoded by a polynucleotide according to the second aspect of the invention, and comprises a nucleotide insertion, deletion or substitution at one or more of positions 3394, 3364, 3354, 3352 and 2205 of AP000510 (corresponding to positions 26724, 26694, 26684, 26675-26682 and 25534 of FIG. 2). The SEEK1 protein or protein fragments of the invention may comprise one or more additional polymorphisms.

The amino acid sequence exactly as shown in FIG. 3(i) may be referred to as the reference sequence, and is not part of the invention. The amino acid sequence of FIG. 3(i) having an amino acid substitution, deletion or insertion at one or more of the positions indicated above may be referred to as a variant of FIG. 3(i). The reference amino acid at one or more of the above polymorphic sites may be replaced by any other amino acid residue to produce a variant sequence. Amino acid sequences of FIG. 3(i) having one or more of the polymorphisms disclosed in Table 1 (i) or Table 1 (iii) are each preferred embodiments of the invention.

Protein fragments may be functional or non-functional and may be useful in drug screening or gene therapy. Functional fragments may be defined as those which have binding and/or immunological characteristics of the SEEK1 protein. The fragments may be at least 10, preferably at least 15, 20, 25 30, 35, 40 or 50 amino acids in length.

In a sixth aspect of the present invention, there are provided antibodies which react with an antigen of a protein or protein fragment of the fifth aspect. A preferred antibody for use in the present invention is one which binds to the amino acid sequence: NH₂-Met-Ile-Ser-Lys-Glu-Phe-His-Leu-Ala-Ala-The-Gln-Asp-Asp-Lys-COOH (SEQ ID NO: 14). Antibodies can be made by the procedure set forth by standard procedures (Harlow and Lane, “Antibodies; A Laboratory Manual” Cold Spring Harbor Laboratory, Cold Spring Harbor, N.Y., 1998). Briefly, purified antigen can be injected into an animal in an amount and in intervals sufficient to elicit an immune response. Antibodies can either be purified directly, or spleen cells can be obtained from the animal. The cells are then fused with an immortal cell line and screened for antibody secretion. The antibodies can be used to screen DNA clone libraries for cells secreting the antigen. Those positive clones can then be sequenced as described in, for example, Kelly et al., Bio/Technology 10:163-167 (1992) and Bebbington et al., Bio/Technology 10:169-175 (1992). The antibody may be specific for the amino acid sequence in question. Preferably, the antibody is sufficiently specific to distinguish between the reference SEEK1 protein and variants thereof, such as those of the fifth aspect. Such antibodies will have use in the first aspect of the invention. Preferably, the antigen being detected and/or used to generate a particular antibody will include proteins or protein fragments according to the fifth aspect.

In a seventh aspect of the present invention, there is provided host cell comprising a polynucleotide according to any of the aforementioned aspects, for expression of the polynucleotide. The host cell may comprise an expression vector, or naked DNA encoding said polynucleotides. A wide variety of suitable host cells are available, both eukaryotic and prokaryotic. Examples include bacteria such as E. coli, yeast, filamentous fungi, insect cells, mammalian cells, preferably immortalised, such as mouse, CHO, HeLa, myeloma or Jurkat cell lines, human and monkey cell lines and derivatives thereof. Such host cells are useful in drug screening systems to identify agents for use in diagnosis or treatment of individuals having, or being susceptible to SEEK1 mediated disease.

The method by which said polynucleotides are introduced into a host cell will usually depend upon the nature of both the vector/DNA and the target cell, and will include those known to a person skilled in the art. Suitable known methods include fusion, conjugation, transfection, transduction, electroporation or injection, as described in Sambrook et al.

In an eighth aspect of the present invention, there is provided a transgenic non-human animal comprising a polynucleotide according to an aforementioned aspect of the invention. Preferably, the transgenic, non-human animal comprises a polynucleotide according to the second or third aspects. Transgenic non-human animals are useful for the analysis of the single nucleotide polymorphisms and their phenotypic effect. Expression of a polynucleotide of the invention in a transgenic non-human animal is usually achieved by operably linking the polynucleotide to a promoter and/or enhancer sequence, preferably to produce a vector of the invention, and introducing this into an embryonic stem cell of a host animal by microinjection techniques (Hogan et al., A Laboratory Manual, Cold Spring harbour and Capecchi Science (1989) 244: 1288-1292). The transgene construct should then undergo homologous recombination with the endogenous gene of the host. Those embryonic stem cells comprising the desired polynucleotide sequence may be selected, usually by monitoring expression of a marker gene, and used to generate a non-human transgenic animal. Preferred host animals include mice and other rodents.

In a preferred embodiment, the transgenic non-human animal may comprise an anti-sense nucleic acid sequence of the fourth aspect. The expression of an anti-sense sequence in a transgenic non-human animal may be useful in determining the effects of such sequences in treating SEEK1-mediated disease, or in neutralising deleterious effects of variant SEEK1 genes in an animal. Preferably, the host animal will be one which suffers from SEEK1 mediated disease. The disease may be naturally occurring or artificially introduced.

In some preferred embodiments, for example where the mediated disease has been artificially induced, the transgenic non-human animal will be modulated to no longer expresses the endogenous SEEK1 gene. Such animals may be referred to as “knock out”. In some cases, it may be appropriate to modulate the expression of the endogenous SEEK1 gene, or express the polynucleotides of the present invention, in specific tissues. This approach removes viability problems if the expression of a gene is abolished or induced in all tissues. Preferably, the specific tissue would be skin.

In a ninth aspect of the present invention there is provided a method of screening for agents for use in the prognosis, diagnosis or treatment of individuals having, or being susceptible to, SEEK1 mediated disease, said method comprising contacting a putative agent with a polynucleotide or protein according to an aforementioned aspect of the present invention, and monitoring the reaction there between. Preferably, the method further comprises contacting a putative agent with a reference polynucleotide (or fragment thereof as described above) or protein of the consensus sequence in FIG. 2 and FIG. 3(i) respectively, and comparing the reaction between (i) the agent and the reference polynucleotide or protein and (ii) the agent and polynucleotide or protein of the invention. Potential agents are those which react differently with a variant of the invention and a reference allele. It is envisaged that the present method may be carried out by contacting a putative agent with a host cell or transgenic non-human animal comprising a polynucleotide or protein according to the invention. Putative agents will include those known to persons skilled in the art, and include chemical or biological compounds, such as anti-sense polynucleotide sequences, complementary to the coding sequences of the second aspect, or polyclonal or monoclonal antibodies which bind to a product such as a protein or protein fragment of the fifth aspect. The agents identified in the present method may be useful in determining susceptibility to SEEK1 mediated disease, or in the diagnosis, prognosis or treatment of said disease.

In a tenth aspect of the invention, there is provided a method for diagnosing and treating SEEK1 mediated disease in a subject, wherein the method comprises

(i) determining which allele of one or more of the polymorphisms of the invention is present; and
(ii) introducing into the subject a different allele;
wherein either a variant allele of the present invention is determined, and/or a variant allele of the present invention is introduced into the subject.

In an alternative embodiment of this aspect, there is provided the use of an allele of one or more polymorphisms of the invention in the manufacture of a medicament for use in the diagnosis and treatment of SEEK1 mediated disease, wherein the method comprises

(i) determining which allele of one or more polymorphisms of the invention are present; and
(ii) introducing into the subject a different allele;
wherein either a variant allele of the present invention is determined, and/or a variant allele of the present invention is introduced into the subject. The medicament may therefore comprise either the variant allele of one or more polymorphisms or the reference allele. In the present invention, treatment includes amelioration of disease.

This method of diagnosis and treatment may comprise determining and introducing alleles in the form of a polynucleotide or protein. In the above embodiments, the allele of a polymorphism may be determined using any method, such as those of the first aspect discussed above. The other allele may be introduced in the form of a protein, or polynucleotide. Any suitable means for introduction of a protein may be used. Introduction of a polynucleotide may use gene therapy methods including those known in the art. In general, a polynucleotide encoding the allele will be introduced into the target cells of a subject, usually in the form of a vector and preferably in the form of a pharmaceutically acceptable carrier. Any suitable delivery vehicle may be used, including viral vectors, such as retroviral vector systems which can package a recombinant genome. The retrovirus could then be used to infect and deliver the polynucleotide to the target cells. Other delivery techniques are also widely available, including the use of adenoviral vectors, adeno-associated vectors, lentiviral vectors, pseudotyped retroviral vectors and pox or vaccinia virus vectors. Liposomes may also be used, including commercially available liposome preparations such as Lipofectin®, Lipofectamine®, (GIBCO-BRL, Inc. Gaitherburg, Md.), Superfect® (Qiagen Inc, Hilden, Germany) and Transfectam® (Promega Biotec Inc, Madison Wis.).

The polynucleotide or vehicle may be administered parenterally (eg, intravenously), transdermally, by intramuscular injection, topically or the like. As SEEK1 mediated diseases are usually manifested in the skin, topical administration is preferred. The exact amount of polynucleotide or vehicle to be administered will vary from subject to subject and will depend upon age, weight, general condition, and severity or mechanism of the disorder.

In a further aspect, the present invention provides a kit for the detection in a subject of a single nucleotide polymorphism according to the present invention. Preferably, the kit will contain polynucleotides according to the aforementioned aspects, most preferably the anti-sense sequences of the third aspect for use as probes or primers; antibodies of the sixth aspect; or restriction enzymes for use in detecting the presence of a polynucleotide, protein or protein fragment of the invention. Preferably, the kit will also comprise means for detection of a reaction, such as nucleotide label detection means, labelled secondary antibodies or size detection means. In yet a further preferred embodiment, the polynucleotides, or antibodies may be fixed to a substrate, for example an array, as described in WO95/11995.

The preferred embodiments of each aspect apply to the other aspects of the invention, mutatis mutandis.

The present invention will now be described by way of a non-limiting example, with reference to the following figures in which:

FIG. 1 shows the nucleotide sequence of the mRNA clone of the SEEK1 gene, of GenBank Accession No. AB031479 (SEQ ID NO: 1) and the encoded protein sequence (SEQ ID NO: 2). On this figure are indicated:
The start and stop codons, atg and taa respectively, underlined at positions 275 nt and 731 nt.
The sites of amino acid polymorphisms (in bold shading) at positions 24, 34, 37 and 133.
The run of polymorphic cytosine residues (in bold shading) starting at nucleotide position 386.

FIG. 2 shows a consensus genomic DNA sequence of SEEK1 (SEQ ID NO:4).

FIG. 3(i) shows the amino acid sequence of the reference SEEK1 protein (SEQ ID NO:5).

FIG. 3 (ii) shows the amino acid sequence of the variant SEEK1 protein with polymorphism SEEK1C3352Del[C]₆ and coding sequence therefore (SEQ ID NO:6). The deletion of a cytosine residue causes a frame shift mutation and premature truncation of the predicted protein—the altered amino acids, relative to the wild type sequence, are shaded.

FIG. 3 (iii) shows the amino acid sequence of the variant SEEK1 protein with polymorphism SEEK1C3352Ins[C]₈ and coding sequence therefore (SEQ ID NO:7). The insertion of a cytosine residue causes a frame shift mutation and premature truncation of the predicted protein—the altered amino acids, relative to the wild type sequence, are shaded.

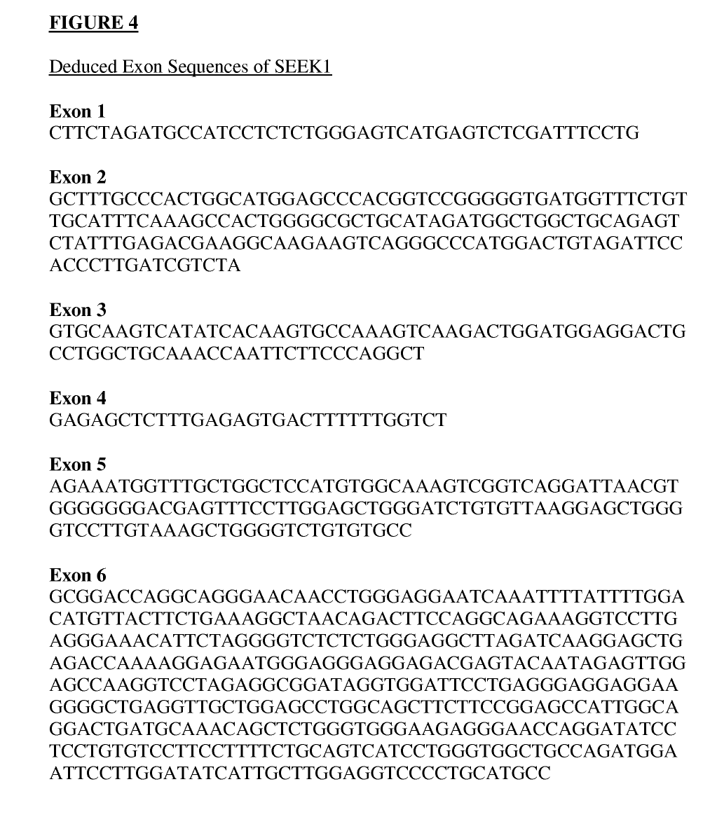

FIG. 4 shows the deduced exonic sequences of SEEK1 (SEQ ID NOS:8-13).

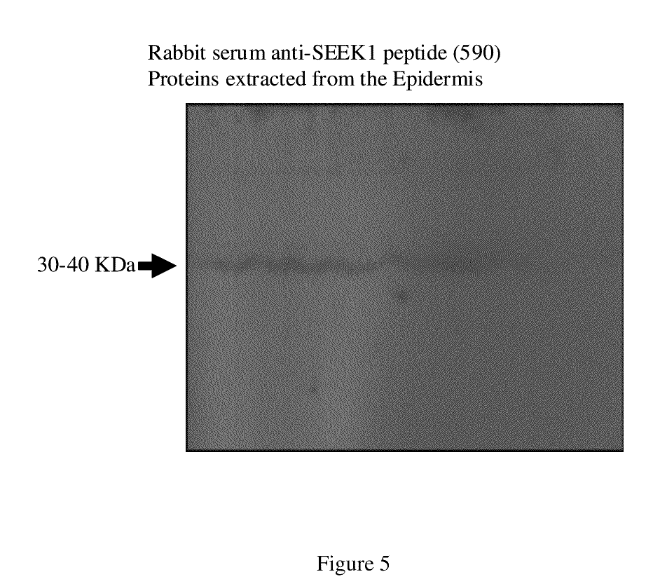

FIG. 5 shows the western blot of epidermal proteins probed with rabbit anti-IgG.

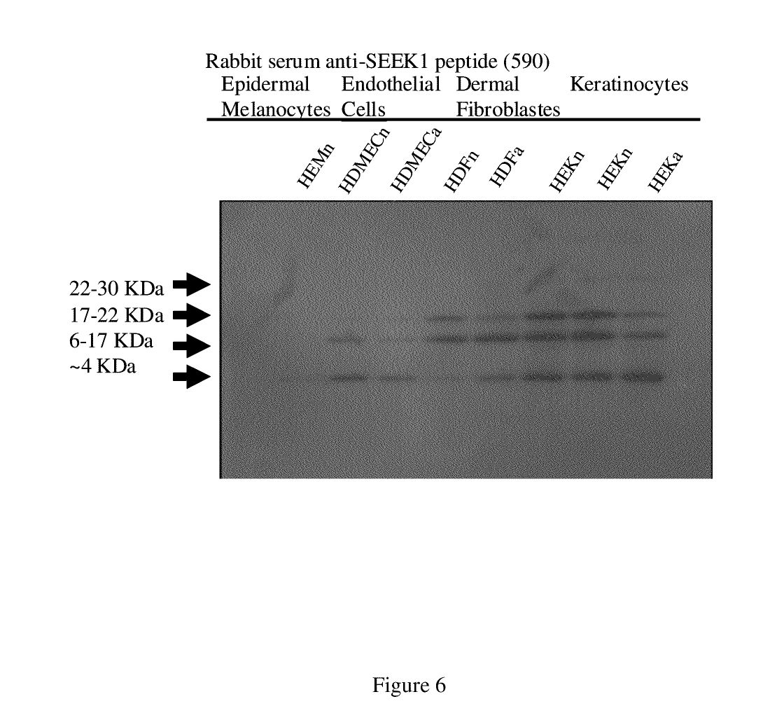

FIG. 6 shows a western blot of proteins of normal human skin cells probed with anti-SEEK1 peptide 590 serum.

### EXAMPLES

### Determination of Gene Structure

The mRNA sequence of the SEEK1 gene (GenBank Accession ID AB031479) was used to screen the following public DNA databases: (available through the National Centre for Biotechnology Information website); NR (Non-Redundant DNA), HTGS (High Throughput Genomic Sequence), dbEST (Expressed Sequence Tag) and GSS (Genome Survey Sequence). The analysis was performed using the BLASTN algorithm (Altschul, et al., (1990) J. Mol. Biol. 215:403-410). Any genomic sequences containing the SEEK1 gene were identified by their degree of sequence identity. The gene structure was determined by comparison of the mRNA sequence with the genomic clones. The deduced exon-intron organization of the SEEK1 gene is presented in FIG. 4. The exon locations in the consensus are as follows:

| | Consensus Genomic |
|---|---|
| SEEK Gene | DNA Position bp |
| Exon 1 | 50586-50541 |
| Exon 2 | 39754-39595 |
| Exon 3 | 35841-35765 |
| Exon 4 | 26978-26948 |
| Exon 5 | 26751-26627 |
| Exon 6 | 25765-25345 |

### Oligonucleotide Primer Design for SEEK1 Gene Sequencing

6 pairs of oligonucleotide primers (Seek1×2F/Seek1×2R; Seek1×3F/Seek1×3R; Seek1×4/5F/Seek1×4/5R; Seek1×6F/Seek1×6R; Seek1×5F/Seek1×5R; Seek1×4F/Seek1×4R—Table 1) were designed to amplify exons 1 to 6 of the SEEK1 gene. Oligonucleotide primer sequences were derived from human chromosome 6p21 genomic DNA sequence (GenBank Accession AP000510).

| | | |
|---|---|---|
| Oligo Name | Sequence 5′ to 3′ | SEQ ID NO: |

| | | |
|---|---|---|
| Seek1x2 F | DAGGTGTTCCGAACATCTCTGC | 16 |
| Seek1X2 R | DACAGCCTGGACACATTCTTCC | 17 |
| Seek1x3 F | DAAGACAGCCTGTTTGAGTGC | 18 |
| Seek1x3 R | DTGTATCCCTTCCTTCTCTCC | 19 |
| Seek1x4/5 F | DAAAGGTAAGAGGTGGTGAGG | 20 |
| Seek1x4/5 R | DATCTGGCTCACCAGAAATGG | 21 |
| Seek1x6 F | DTTTCAAACCTGGGATGCAGC | 22 |
| Seek1x6 R | DAGATGAGATCACGCCATTGC | 23 |
| Seek1x4 F | DATGCCTGTAAAGGAGGAAGG | 24 |
| Seek1x4 R | DAAAGTGGGTCAAGTGAACGG | 25 |
| Seek1x5 F | DTAAGCTCCATCCACCCCTGG | 26 |
| Seek1x5 R | DAACTGGACGCATGGGGTTGG | 27 |
| Seek1x6 F2 | DATGGGATCCAGGCATCCTGC | 28 |
| Seek1x6 R2 | DTTTGGACAGGGTGTGGAGGG | 29 |

### SEEK1 Gene Amplification

Genomic DNA from a panel of 24 unrelated individuals was amplified using primer pairs (Seek1×2F/Seek1×2R; Seek1×3F/Seek1×3R; Seek1×4/5F/Seek1×4/5R; Seek1×6F/Seek1×6R; Seek1×5F/Seek1×5R; Seek1×4F/Seek1×4R—Table 1). 100 ng genomic DNA was amplified by PCR in a total reaction volume of 25 μl containing 50 mM KCl, 20 mM Tris.HCl (pH 8.4), 2 mM MgCl₂ 200 μM each dATP, dCTP, dGTP, dTTP, 1 μM each oligonucleotide primer and 0.5 units AmpliTaq Gold DNA polymerase (Applied Biosystems). Reactions were thermocycled with an initial denaturation step of 95° C./10 mins followed by 35 cycles of 94° C./30 secs; Tₘ annealing/30 secs; 72° C./30 secs. A final elongation step of 72° C./10 mins completed the amplification.

TABLE 2
Primers and Amplimer Sizes.

| | Forward | Reverse | Product |
|---|---|---|---|
| Fragment | Primer | Primer | size (bp) |
| Exon 2 | Seek1x2 F | Seek1x2 R | 402 |
| Exon 3 | Seek1x3 F | Seek1x3 R | 294 |
| Exon 4 and 5 | Seek1x4/5 F | Seek1x4/5 R | 531 |
| Exon 6 | Seek1x6 F | Seek1x6 R | 627 |
| Exon 5 | Seek1x5 F | Seek1x5 R | 340 |
| Exon 4 | Seek1x4 F | Seek1x4 R | 241 |

### Heteroduplex Analysis Using DHPLC:

Oligos were designed to amplify products of between 241-627 bp in length from the genomic DNA of 24 individuals. Denaturing high-performance liquid chromatography (DHPLC) analysis was performed using the WAVE™ DNA fragment analysis system (Transgenomic) (Kuklin, et al, (1997-98) Genet Test. 1(3): 201-6.). The temperature required for successful resolution of heteroduplex molecules within each PCR product was determined empirically by injecting PCR product at a series of increasing mobile phase temperatures and constructing a fragment specific melting curve. A universal gradient for double stranded DNA was used to determine the appropriate acetonitrile concentration for the heteroduplex identification. For mutation detection, 1-2 μl aliquots of the PCR reactions from each of the eleven individuals were injected onto the WAVE™ column. Mutation detection gradients were for four minutes. Results were graphically visualised using the D-7000 HSM software (Transgenomic).

### Direct Sequencing of PCR Products

To define the exact nature of the polymorphisms identified by DHPLC heteroduplex analysis, 50-100 ng of PCR products were sequenced in both orientations using the DYEnamic ET terminator cycle sequencing premix kit from Amersham. Reactions were fractionated on ABI 377 automated sequencers using standard procedures. Chromatographic traces were analysed using the SEQUENCHER programme (Gene Codes, USA), to identify SNP positions.

The single nucleotide polymorphisms of the SEEK1 gene, including those of the present invention, are listed in Tables 1 (i) and (ii) where:
Column 1 of (i) provides the name and positional reference of the polymorphism with respect to the reference genomic DNA sequence AP000510, together with details of the polymorphism itself. For example, the reference “G16549A” indicates a substitution of the nucleotide “G” for nucleotide “A” at position 16549 of AP000515.
Column 2 of (i) provides the positional reference of the polymorphism with respect to the reference mRNA sequence AB031479.
Column 3 of (i) shows the sequence flanking the polymorphism, the polymorphism itself being shown in underlined type.
Column 4 of (i) shows the IUB code of each single nucleotide polymorphism.
Columns 5 and 6 of (i) shows the effect of each single nucleotide polymorphism on the amino acid sequence of SEEK1.
Columns 2 and 3 of (ii) show the forward and reverse primers which may be used to amplify a region of the SEEK1 gene to enable detection of the single nucleotide polymorphisms detailed in Column 1 of (ii).
Column 4 of (ii) shows the size in base pairs of the amplified products.

The single nucleotide polymorphisms of the SEEK1 gene, as set out in Tables (i) and (ii) are also listed in Table 3(iii) which includes additional single nucleotide polymorphisms. In Table 3:
Column 1 provides SNP number.
Column 2 provides the SNP location in the SEEK1 gene.
Column 3 shows the flanking sequence of the polymorphism, the polymorphism itself being underlined.
Column 4 shows the IUB code of each single nucleotide polymorphism.
Column 5 shows the nucleotide position in AB031479 (see FIG. 1).
Column 6 shows any resulting amino acid polymorphism.
Column 7 shows the nucleotide position in the consensus genomic DNA sequence (FIG. 2).
Columns 8 and 9 show statistical significance of the polymorphism association.

### Detection of Polymorphisms in 24 Population Controls

Allele frequencies of the SEEK1C3352Ins/Del[C] polymorphism were determined in 24 population controls by direct DNA sequencing of PCR products generated using primers Seek1×5F and Seek1×5R.

### SEEK1C3352Del [C]₆—16.5%

### SEEK1C3352WT[C]₇—74.0%

### SEEK1C3352Ins [C]₈—9.50%

### Production of an Antibody to SEEK1 Protein

The following peptide (SEQ ID NO:15), termed 590-THY, was synthesised and coupled to thyroglobulin for SEEK1 anti-sera production.

### NH₂-Met-Ile-Ser-Lys-Glu-Phe-His-Leu-Ala-Ala-Thr-Gln-Asp-Asp-Cys-COOH

Two rabbits were immunised with peptide 590-THY for polyclonal production. Two immunisations are performed at 4 weeks intervals with two sample bleeds for testing

### ELISA

Sera from the two immunised rabbits and four mice were tested for their reactivity to the peptide 590. Each time the controls (rabbit and mice not immunised) were tested at different (serial) dilutions. The optimum dilution was approximately 1:10000. Control serum was negative.

### Western Blotting

Proteins were extracted from the epidermis. Electrophoresis and transfer were performed according to standard techniques. The blotting membrane was incubated with the primary antibody 590 (1:1000) then with second antibody, rabbit anti-IgG, coupled to biotin. Detection was performed using ECL-plus reagents (Pharmacia). A band was detected at approximately 40 kDa, possibly representing a glycosylated form of the protein (FIG. 5).

Western blotting was also performed with proteins extracted from several normal human skin cell lines including neonatal keratinocytes (NHEK-Neo), adult keratinocytes (NHEK-Ad), neonatal pooled, neonatal dermal fibroblasts (NHDF-Neo) and adult dermal fibroblasts (NHDF-Ad), neonatal dermal microvascular endothelial cells (HMVEC-Neo) and adult), dermal microvascular endothelial cells (HMVEC-Ad) and epidermal melanocytes (NHEM-Neo). Four bands were observed at molecular weights 22-30 kDa, 17-22 kDa, 6-17 kDa and 4 kDa (FIG. 6).

SEEK1 Gene Association with Psoriasis

SEEK 1 gene polymorphisms were genotyped in 147 families identified through a proband with psoriasis (a total of 499 individuals, of whom 233 were affected). Genotyping was performed using Pyrosequencing (Ahmadian A et al., Anal Biochem 2000 280:103-110), an established genotyping technology well known to those skilled in the art.

### Single Point Association

Single point associations between each polymorphism and psoriasis affected status were calculated using the TRANSMIT program (Clayton D, MRC Biostatistics Unit, Cambridge). P values <0.05 and corresponding chi squared values are provided in Table 1(iii). Highly significant associations were observed between SNPs 16, 17, 19, 20, 21, 22, 23, 24, 25, 40, 46, 56 and psoriasis. The single SNP showing the most significant association with psoriasis is SNP 24. There are no published data reporting the association of SEEK1 gene SNPs and psoriasis. This study has identified at least 12 SNPs that are powerfully predictive of affected status.

TABLE 1

| (i) | | | | | | |
|---|---|---|---|---|---|---|
| SNP position | AB031479 | SEQ | | | | |
| and details | nt | ID | | IUB | | Amino Acid |
| SNP name | position | NO: | SNP | Code | Effect | Position |
| G16549A | n/a | 30 | CCCCAATCAGGTGTTCCGAACATCTCTGCG G/A | R | Non-coding | |
| | | | [G/A]GACTGACCCTCCTCAGCCCAGGTGCTCC[C/T] | | | |
| G16548A | n/a | 31 | CCCAATCAGGTGTTCCGAACATCTCTGCG[G/A] G/A | R | Non-coding | |
| | | | GACTGACCCTCCTCAGCCCAGGTGCTCC[C/T]A | | | |
| C16519T | n/a | 32 | [G/A][G/A]GACTGACCCTCCTCAGCCCAGGTGCTCC C/T | Y | Non-coding | |
| | | | ATGGGACTGGCTACACTTCTTGACTCAGTT | | | |
| A16393G | n/a | 33 | GTAGACGATCAAGGGTGGAATCTACAGTCC A/G | R | Non-coding | |
| | | | TG[G/A]GCCCTGACTTCTTGCCTTC[G/A]TCTCAAA | | | |
| G16390A | n/a | 34 | GACGATCAAGGGTGGAATCTACAGTCC[A/G]TG G/A | R | Non-coding | |
| | | | GCCCTGACTTCTTGCCTTC[G/A]TCTCAAATAG | | | |
| G16370A | n/a | 35 | ACAGTCC[A/G]TG[G/A]GCCCTGACTTCTTGCCTTC G/A | R | Non-coding | |
| | | | TCTCAAATAGACTCTGCAGCCAGCCATCTA | | | |
| C12553T | n/a | 6 | ATTAATAGGTACTAAAATCTCCAATTGCCT C/T | Y | Non-coding | |
| | | | ATGCCTCCCCCTTCTCTTTCCCACTCACCT | | | |
| G12401T | n/a | 37 | GTGAGTTACCTCTCTCAGTGTTGGTTCCTC G/T | K | Non-coding | |
| | | | TCTGTGAAATGGGGCTAATCATTTGCTTTA | | | |
| T3676C | n/a | 38 | CCCAGCCCCAGGAGGAGGAGCCTGTCTGGA T/C | Y | Non-coding | |
| | | | GGACGCAGCCTGAACTGACCCACAAACAGA | | | |
| A3585G | n/a | 39 | TAGGTTTGTAAATACTTAACTGATGGTAAA A/G | R | Non-coding | |
| | | | TGTCATGAACCCCTACCCCCGATGGATCTG | | | |
| A3444G | n/a | 40 | GCTTTGTCCTCAGGCCAACCTGCAACCCAA A/G | R | Non-coding | |
| | | | GTGGGTTACACCTTGGCCCCCAGGCACACA | | | |
| C3394A | 344 | 41 | CCAGGCACACAGACCCCAGCTTTACAAGGA C/A | M | Amino acid | Pro24Thr |
| | | | CCCAGCTCCTTAACACAGATCCCAGCTCC[G/A] | | substitution | |
| G3364A | 374 | 42 | [C/T]CCCAGCTCCTTAACACAGATCCCAGCTCC G/A | R | Amino acid | Lys34Glu |
| | | | AGGAAACTC[G/A]T[:CCCCCCC]ACGTTAATCCT | | substitution | |
| G3354A | 384 | 43 | TTAACACAGATCCCAGCTCC[G/A]AGGAAACTC G/A | R | Amino acid | Arg37His |
| | | | T[:CCCCCCC]ACGTTAATCCTGACCGACTTT | | substitution | |
| C3352Ins/Del[ | 386 | 44 | AACACAGATCCCAGCTCC[G/A]AGGAAACTC[G/A]T (C)7[wild | n/a | (C)7 - wild | See FIG. 2 |
| | | | type] ACGTTAATCCTGACCGACTTTGCCACATGG | | type peptide | (i) |
| C3352Ins/Del[ | 386 | 45 | AACACAGATCCCAGCTCC[G/A]AGGAAACTC[G/A]T (C)6[DEL] | n/a | (C)6- | See FIG. 2 |
| | | | ACGTTAATCCTGACCGACTTTGCCACATGG | | truncated | (ii) |
| | | | | | peptide | |
| C3352Ins/Del[ | 386 | 46 | AACACAGATCCCAGCTCC[G/A]AGGAAACTC[G/A]T (C)8[INS] | n/a | (C)8- | See FIG. 2 |
| | | | ACGTTAATCCTGACCGACTTTGCCACATGG | | truncated | (iii) |
| | | | | | peptide | |
| A3247T | n/a | 47 | TCTGCACCATGTCCCCCACCCAATGTGTCC A/T | W | Non-coding | |
| | | | GAAAGCCATTTCTGGTGAGCCAGATGCACC | | | |
| G3210A | n/a | 48 | CATTTCTGGTGAGCCAGATGCACCTTCTGC G/A | R | Non-coding | |
| | | | TCCCCTGAATTCCTGTCCCCAACCCCATGC | | | |
| C2205T | 673 | 49 | TCCACCTATCCGCCTCTAGGACCTTGGCTC C/T | Y | Amino acid | Pro133Leu |
| | | | AACTCTATTGTACTCGTCTCCTCCCTCCCA | | substitution | |
| C2126Del[C] | 748 | 50 | CTCCTTGATCTAAGCCTCCCAGAGAGACCC C[INS/DEL] | n/a | 3′ UTR | |
| | | | TAGAA[C/T]GTTTCCCTCAAGGACCTTTCTGCC | | | |
| C2120T | 757 | 51 | GATCTAAGCCTCCCAGAGAGACCC[C]TAGAA C/T | Y | 3′ UTR | |
| | | | GTTTCCCTCAAGGACCTTTCTGCCTGGAAG | | | |

TABLE 1
(ii)
Primers for SNP
amplification

| | Forward | Reverse | Product |
|---|---|---|---|
| SNP name | Primer | primer | size (bp) |
| G16549A | Seek1x2 F | Seek1x2 R | 402 |
| G16548A | Seek1x2 F | Seek1x2 R | 402 |
| C16519T | Seek1x2 F | Seek1x2 R | 402 |
| A16393G | Seek1x2 F | Seek1x2 R | 402 |
| G16390A | Seek1x2 F | Seek1x2 R | 402 |
| G16370A | Seek1x2 F | Seek1x2 R | 402 |
| C12553T | Seek1x3 F | Seek1x3 R | 294 |
| G12401T | Seek1x3 F | Seek1x3 R | 294 |
| T3676C | Seek1x4 F | Seek1x4 R | 241 |
| A3585G | Seek1x4 F | Seek1x4 R | 241 |
| A3444G | Seek1x5 F | Seek1x5 R | 340 |
| C3394A | Seek1x5 F | Seek1x5 R | 340 |
| G3364A | Seek1x5 F | Seek1x5 R | 340 |
| G3354A | Seek1x5 F | Seek1x5 R | 340 |
| C3352Ins/Del[C] | Seek1x5 F | Seek1x5 R | 340 |
| SEEK1x5.A265T | Seek1x5 F | Seek1x5 R | 340 |
| SEEK1x5.G302A | Seek1x5 F | Seek1x5 R | 340 |
| SEEK1x6.C273T | Seek1x6 F2 | Seek1x6 R2 | 627 |
| SEEK1x6.C352Del[C] | Seek1x6 F2 | Seek1x6 R2 | 627 |
| SEEK1x6.C358T | Seek1x6 F2 | Seek1x6 R2 | 627 |

TABLE 1 (iii)

| | | | | | | | Nucleotide | | |
|---|---|---|---|---|---|---|---|---|---|
| | | SEQ | | | Nucleotide | | position in | | Chi |
| | SNP Location | ID | | SNP IUB | position in | Amino Acid | consensus genomic | | squared |
| SNP | in SEEK gene | NO: | Sequence context of SNP | Code | AB031479 | Polymorphism | DNA sequence | p value | value |
| 1 | Promoter | 52 | GAAATAGCCACYTTCTCCCAAGGTTTCTTATACTCTRTGGCACATCTGACCACCAGT | R | N/A | N/A | 51814 | | |
| | | | AGCAGGCAGAATGATGT | | | | | | |
| 2 | Promoter | 53 | CTCCTCTACTGTTACTTGGAAATAGCCACYTTCTCCCAAGGTTTCTTATACTCT | Y | N/A | N/A | 51789 | | |
| 3 | Promoter | 54 | GATCAAGTCCTGGCCATTTGACAGCAGCATTTAAAGGCYCTCCTCTACTGTTACTTG | Y | N/A | N/A | 51759 | | |
| | | | GAAATAGCCACYTTCTCCCAAGGT | | | | | | |
| 4 | Promoter | 55 | CATGTTTAGACCTTGGGCAGCCAGGGAAGCYTACTCCTGGGGCCTCCCGGAAGCC | Y | N/A | N/A | 51570 | | |
| | | | ATGGAGAGAAC | | | | | | |
| 5 | Promoter | 56 | CTCTTCACTCCTCCAGTGGTTAAGCCAGCAGGGGCAGGYGGGGAGGACACAGCAG | Y | N/A | N/A | 51505 | | |
| | | | TAGAATCAGCCAACAGCTCAT | | | | | | |
| 6 | Promoter | 57 | AGGCCTCTGGGCTCCATCCACTGCCAGTTCTGGAGWGGAGCTCTTCACTCCTCCA | W | N/A | N/A | 51462 | | |
| | | | GTGGTTAAGCCAGCA | | | | | | |
| 7 | Promoter | 58 | ACATTGACCAGAAAGGGATTGAATCACCCTTGGTCCAGCRTCTGGCCCCTGATCTG | R | N/A | N/A | 51265 | | |
| | | | CAGCCAATGGCAGGAATCGAGGTC | | | | | | |
| 8 | Promoter | 59 | TGAATTTAGAACTGTTGAAACTCCAAGTCTGGAATCAGCARAAATGTATTACATTGAC | R | N/A | N/A | 51216 | | |
| | | | CAGAAAGGGATTGAATCACCCT | | | | | | |
| 9 | Promoter | 60 | CTCAGAGCCTCTGCTTGGCTGCAAAGGAATTCACCCYTACTGTAGCACTTAACCCAT | Y | N/A | N/A | 51124 | | |
| | | | TCCCTCCTATCAGGGTGG | | | | | | |
| 10 | Promoter | 61 | GGATTGTGCTTGTCCCTGTAGGAGCCCCACCCCCCACCCYAGGCCACCTCTCAGA | Y | N/A | N/A | 51078 | | |
| | | | GCCTCTGCTTGGCTGCAAAGG | | | | | | |
| 11 | Promoter | 62 | TGAGACAGGCAGGGAGAGGCTGAGGCGGASGAAGTTCCYGCATCCCAAGGAGGG | Y | N/A | N/A | 51017 | | |
| | | | CAGAGTGGATTGTGCTTGTCC | | | | | | |
| 12 | Promoter | 63 | GACTTAAGTCCTGAGACAGGCAGGGAGAGGCTGAGGCGGASGAAGTTCCYGCATC | S | N/A | N/A | 51008 | | |
| | | | CCAAGGAGGGCAGAGTGGATT | | | | | | |
| 13 | Promoter | 64 | GCTGAGAAGGCAGAGTGCCCCMGTGGGAAAGAGGAGTCGCYTCCACTGGAGAAG | Y | N/A | N/A | 50920 | | |
| | | | AGAGAGAAAGTGGAGTGTGTGGTG | | | | | | |
| 14 | Promoter | 65 | AACATGGCTCTCAGGTGAGGGCTGAGAAGGCAGAGTGCCCCMGTGGGAAAGAGG | M | N/A | N/A | 50901 | | |
| | | | AGTCGCYTCCACTGGAGAAGAGAGA | | | | | | |
| 15 | Promoter | 66 | TAGATCAAGAGGCCCAGCCTGTGGCAGAACAGAGCTGCCRGTGGTCTCTCCATCTT | R | N/A | N/A | 50801 | | |
| | | | CACACTCCCTGCTCTGCTGGGGT | | | | | | |
| 16 | Intron 1 | 67 | TCTCAGCCCCTTCCTGTGGCCATTTCCCTCAGTGCYCAGATGATTCCCTGGGTGAG | Y | N/A | N/A | 50049 | 0 | 26.13 |
| | | | GGAGACACTGGGGCACCCTC | | | | | | |
| 17 | Intron 1 | 68 | TACCCCAAGGAGAGTTACTCGACAGTCCAT[AAG]AAGTCAACTGTTGTGTGTGTGCA | INS/DEL | N/A | N/A | 49405-49407 | 0 | 18.31 |
| | | | TGCCTTGGGCACAAA | | | | | | |
| 18 | Intron 1 | 69 | AGTTCCCAATCSAGTGGCAAAATCATCCTTCAGCCTTGYGGCAGCAAGTCCAGCTC | Y | N/A | N/A | 49160 | | |
| | | | TTCTGGTCACCCTTGC | | | | | | |
| 19 | Intron 1 | 70 | GGCACCGGCTCCTTCAGCAGCAGCTCCAGTTCCCAATCSAGTGGCAAAATCATCCT | S | N/A | N/A | 49133 | 0.001 | 11.01 |
| | | | TCAGCCTTGYGGCAGCAA | | | | | | |
| 20 | Intron 1 | 71 | CCAGCRGTTCTAGCATTTCCAGCAGCKCCGGTTYACCCTACCATCCCTGCGGCAGT | Y | N/A | N/A | 49045 | 0.003 | 9.7 |
| | | | GCTTCCCAGAG | | | | | | |
| 21 | Intron 1 | 72 | CTCGAGTCCCCAGCRGTTCTAGCATTTCCAGCAGCKCCGGTTYACCCTACCATCCC | K | N/A | N/A | 49038 | 0.05 | 4.55 |
| | | | TGCGGCAGTGCTT | | | | | | |
| 22 | Intron 1 | 73 | CCAAGGGACCCTGCTCTCCCTCCAGTTCTCGAGTCCCCAGCRGTTCTAGCATTTCC | R | N/A | N/A | 49017 | 0.0008 | 11.16 |
| | | | AGCAGCKCCGGTTYACCCTA | | | | | | |
| 23 | Intron 1 | 74 | ACCCCATCATCCCCAGCCAGTCGGCAGCTTCCTCGGCCATTGCRTTCCAGCCAGTG | R | N/A | N/A | 48920 | 0.0007 | 12.12 |
| | | | GGGACTGGTGGGGTCCAGC | | | | | | |
| 24 | Intron 1 | 75 | CCAGGCATGACCTACAGTAAGGGTAAAATCTAYCCTGTGGGCTACTTCACCAAAGA | Y | N/A | N/A | 48773 | 0 | 36.5 |
| | | | GAACCCTGTGAAAGG | | | | | | |
| 25 | Intron 1 | 76 | CAGGGACCTTGGCTAAGAGCATTGGCACCTTCTCAGACCYCTGTAAGGACCCCACG | Y | N/A | N/A | 47938 | 0.0003 | 12.81 |
| | | | CGTATCACCTCCCCTAACGACCCCT | | | | | | |
| 26 | Intron 1 | 77 | KACTGAGATAAGGCAGAAAGGTGAGGRAGGAAGCCAAGCCTCYTTGGCCCTTACTA | Y | N/A | N/A | 47868 | | |
| | | | ACCACTGCTTTCCTCCACAGGGACCTTG | | | | | | |
| 27 | Intron 1 | 78 | AGAGGCCGATKACTGAGATAAGGCAGAAAGGTGAGGRAGGAAGCCAAGCCTCYTT | R | N/A | N/A | 47852 | | |
| | | | GGCCCTTACTAACCACTG | | | | | | |
| 28 | Intron 1 | 79 | CCCTGCGCTCTGCTTGGGAGAAACCCGAGAGGCCGATKACTGAGATAAGGCAGAA | K | N/A | N/A | 47826 | | |
| | | | AGGTGAGGRAGGAAGCCA | | | | | | |
| 29 | Intron 1 | 80 | TCAATGTATTCCTTTGAGGYCACTCACTTTGGCACSTAATTTTCTATTTTTCTGGTTG | S | N/A | N/A | 47661 | | |
| | | | GTGTTTGCCCACCCTT | | | | | | |
| 30 | Intron 1 | 81 | AGCCCCCTCTTATATTCAATGTATTCCTTTGAGGYCACTCACTTTGGCACSTAATTTT | Y | N/A | N/A | 47645 | | |
| | | | CTATTTTTCTGGTTG | | | | | | |
| 31 | Intron 1 | 82 | TCTTGAACTCTGGGGCRCATGCAATCCTCCCACCTCRGCCTCCCAAAGTGCTGGGA | R | N/A | N/A | 47567 | | |
| | | | TTACCGGCGTGAGCCACT | | | | | | |
| 32 | Intron 1 | 83 | GGGTCTATGTTGCCCAGGCTGGTCTTGAACTCTGGGGCRCATGCAATCCTCCCACC | R | N/A | N/A | 47547 | | |
| | | | TCRGCCTCCCAAAGTGCTGG | | | | | | |
| 33 | Intron 1 | 84 | AAAAAAATTTTAATTAAAAAACAAAATACAGAYRGGGTCTATGTTGCCCAGGCTGGT | R | N/A | N/A | 47508 | | |
| | | | CTTGAACTCTGGGGCRC | | | | | | |
| 34 | Intron 1 | 85 | AAAAAAATTTTAATTAAAAAACAAAATACAGAYRGGGTCTATGTTGCCCAGGCTGGT | Y | N/A | N/A | 47507 | | |
| | | | CTTGAACTCTGGGGCRC | | | | | | |
| 35 | Intron 1 | 86 | CTGTCTCTTCAGGGTCCTTTCTTTTAGACCTAYTTGTTCCTGCCCCTTCTCCATTCCC | Y | N/A | N/A | 47438 | | |
| | | | TCTTCTTTT | | | | | | |
| 36 | Intron 1 | 87 | GGAGGAACCAYGGGGTAAGTTGGGCCTGGGGTTTTSAGCAAAGGAAAGGAAAGAT | S | N/A | N/A | 46831 | | |
| | | | AAGGAAAGATGTGGCTC | | | | | | |
| 37 | Intron 1 | 88 | CAGAAGGAACGCAGGWGAAAGAGTCATGGAGGAACCAYGGGGTAAGTTGGGCCT | Y | N/A | N/A | 46806 | | |
| | | | GGGGTTTTSAGCAA | | | | | | |
| 38 | Intron 1 | 89 | CTGGAGGGGCTAGGGAAGGCAGAAGGAACGCAGGWGAAAGAGTCATGGAGGAAC | W | N/A | N/A | 46784 | | |
| | | | CAYGGGGTAAGTTGGGCCTGG | | | | | | |
| 39 | Intron 1 | 90 | AGGTGTTCCGAACATCTCTGCGRRGACTGACCCTCCTCAGCCCAGGTGCTCCYATG | R | N/A | N/A | 39881 | | |
| | | | GGACTGGCTACACTTCTTGACTCAGTTTTAATCTCTCCTTCTCTGCCTTCCTGTTGG | | | | | | |
| | | | GAATACCCCCTCACTTCTGTGGCTTCTTTCCTGTAGTAGACGATCAAGGGT | | | | | | |
| 40 | Intron 1 | 91 | AGGTGTTCCGAACATCTCTGCGRRGACTGACCCTCCTCAGCCCAGGTGCTCCYATG | R | N/A | N/A | 39880 | 0 | 29.56 |
| | | | GGACTGGCTACACTTCTTGACTCAGTTTTA | | | | | | |
| 41 | Intron 1 | 92 | TCTCTGCGRRGACTGACCCTCCTCAGCCCAGGTGCTCCYATGGGACTGGCTACACT | Y | N/A | N/A | 39851 | | |
| | | | TCTTGACTCAGTTTT | | | | | | |
| 42 | Exon 2 | 93 | TCAAGGGTGGAATCTACAGTCCRTGRGCCCTGACTTCTTGCCTTCRTCTCAAATAGA | R | N/A | N/A | 39725 | | |
| | | | CTCTGCAGCCAGCCATCTATGCAGCGC | | | | | | |
| 43 | Exon 2 | 94 | GGGTGGAATCTACAGTCCRTGRGCCCTGACTTCTTGCCTTCRTCTCAAATAGACTCT | R | N/A | N/A | 39722 | | |
| | | | GCAGCCAGCCATCTATGCAGCGC | | | | | | |
| 44 | Exon 2 | 95 | ATCTACAGTCCRTGRGCCCTGACTTCTTGCCTTCRTCTCAAATAGACTCTGCAGCCA | R | N/A | N/A | 39702 | | |
| | | | GCCATCTATGCAGCGCCCCAGTGGC | | | | | | |
| 45 | Intron 2 | 96 | CCTATTAATAGGTACTAAAATCTCCAATTGCCTYATGCCTCCCCCTTCTCTTTCCCAC | Y | N/A | N/A | 35884 | | |
| | | | TCACCTACCTGCCATGTCAGCC | | | | | | |
| 46 | Intron 3 | 97 | GGCACTTGTGATATGACTTGCACAGGTGAGTTACCTCTCTCAGTGTTGGTTCCTCKT | K | N/A | N/A | 35732 | 0.001 | 11.05 |
| | | | CTGTGAAATGGGGCTAATCATTTGCTTTATTG | | | | | | |
| 47 | Intron 3 | 98 | CAGCCCCACCCAGCCCCAGCCCCAGGAGGAGGAGCCTGTCTGGAYGGACGCAGC | Y | N/A | N/A | 27006 | | |
| | | | CTGAACTGACCCACAAACAGACCAAAAAA | | | | | | |
| 48 | Intron 4 | 99 | ACCAAAAAAGTCACTCTCAAAGAGCTCTCGGTAGGTTTGTAAATACTTAACTGATGG | R | N/A | N/A | 26915 | | |
| | | | TAAARTGTCATGAACCCCTACCCCCGATGGATCTGAACCGTTCACTTGACCCACTTT | | | | | | |
| 49 | Intron 4 | 100 | CACTAGCTTTGTCCTCAGGCCAACCTGCAACCCAARGTGGGTTACACCTTGGCCCC | R | N/A | N/A | 26770 | | |
| | | | CAGGCACACAGACCCCAGCTTTACA | | | | | | |
| 50 | Exon 5 | 101 | TCAGGCCAACCTGCAACCCAARGTGGGTTACACCTTGGCCCCCAGGCACACAGAC | M | 344 | Pro24Thr | 26724 | | |
| | | | CCCAGCTTTACAAGGAMCCCAGCTCCTTAACACAGATCCCAGCTCCRAGGAAACTC | | | | | | |
| | | | GT:CCCCCCCACGTTAATCCT | | | | | | |
| 51 | Exon 5 | 102 | TCACAGACCCCAGCTTTACAAGGAMCCCAGCTCCTTAACACAGATCCCAGCTCCRA | R | 374 | Lys34Glu | 26694 | | |
| | | | GGAAACTCRT:CCCCCCCACGTTAATCCTGACCGACTTTGCCACATGGAGCCAGCAA | | | | | | |
| | | | ACCATT | | | | | | |
| 52 | Exon 5 | 103 | TTAACACAGATCCCAGCTCCRAGGAAACTCRT[:CCCCCCC]ACGTTAATCCTGACCG | R | 384 | Arg37His | 26684 | | |
| | | | ACTTT | | | | | | |
| 53 | Exon 5 | 104 | AACACAGATCCCAGCTCCRAGGAAACTCRT[C]CCCCCCCACGTTAATCCTGACCGA | INS/DEL | 386-392 | See FIG. | 26675-26682 | | |
| | | | CTTTGCCACATGG | | | 2 (i) | | | |
| 54 | Intron 5 | 105 | AGCCAAATGCACCTTCTGCACCATGTCCCCCACCCAATGTGTCCWGAAAGCCATTT | W | N/A | N/A | 26576 | | |
| | | | CTGGTGAGCCAGATGCACCTTCTGCRTCCCCTGAATTCCTG | | | | | | |
| 55 | Intron 5 | 106 | GCACCATGTCCCCCACCCAATGTGTCCWGAAAGCCATTTCTGGTGAGCCAGATGCA | R | N/A | N/A | 26539 | | |
| | | | CCTTCTGCRTCCCCTGAATTCCTGTCCCCAACCCCATGCGTCCAGTT | | | | | | |
| 56 | Exon 6 | 107 | TCCTCCCTCAGGAATCCACCTATCCGCCTCTAGGACCTTGGCTCYAACTCTATTGTA | Y | 672 | Pro133Leu | 25534 | 0.03 | 5.5 |
| | | | CTCGTCTCCTCCCTCCCATTCTCCTTTTGGTC | | | | | | |
| 57 | Exon 6 | 108 | CCTCCCATTCTCCTTTTGGTCTCAGCTCCTTGATCTAAGCCTCCCAGAGAGACCC[C] | INS/DEL | 748 | 3′UTR | 25458 | | |
| | | | TAGAAYGTTTCCCTCAAGGACCTTTCTGC | | | | | | |
| 58 | Exon 6 | 109 | ATTCTCCTTTTGGTCTCAGCTCCTTGATCTAAGCCTCCCAGAGAGACCCCTAGAAYG | Y | 757 | 3′UTR | 25449 | | |
| | | | TTTCCCTCAAGGACCTTTCTGCCTGGA | | | | | | |

1. A method of diagnosing or determining susceptibility to, an inflammatory disease, the method comprising:
identifying a human subject in need of being diagnosed with or determining susceptibility to the inflammatory disease, wherein the inflammatory disease is a SEEK1-mediated disease of the skin;
comparing a portion of a SEEK1 gene or protein of the subject with a SEEK1 gene of SEQ ID NO: 4 or SEEK1 protein of SEQ ID NO:5; and
determining the presence of one or more polymorphisms in the SEEK1 gene or protein of the subject.

2. A method according to claim 1, comprising determining the presence of a nucleotide substitution, deletion or insertion at any one or more of positions 51814, 51789, 51759, 51570, 51505, 51462, 51265, 51216, 51124, 51078, 51017, 51008, 50920, 50901, 50801, 50049, 49405-49407, 49160, 49133, 49045, 49038, 49017, 48920, 48773, 47938, 47868, 47852, 47826, 47661, 47645, 47567, 47547, 47508, 47507, 47438, 46831, 46806, 46784, 39881, 39880, 39851, 39725, 39722, 39702, 35884, 35732, 27006, 26915, 26770, 26724, 26694, 26684, 26675-26682, 26576, 26539, 25534, 25478 and 25449 of the SEEK1 gene of SEQ ID NO:4.

3. A method according to claim 1, said method comprising determining the presence of an amino acid substitution, deletion or insertion at one or more of positions 24, 34, 37, 40 or 133 of the SEEK1 amino acid sequence of SEQ ID NO:5 or the presence of a SEEK1 protein fragment of SEQ ID NO:6 or SEQ ID NO:7.

4. A method of claim 1, wherein the inflammatory disease is psoriasis.

5. A method of diagnosing psoriasis in a human subject, or determining the susceptibility of a human subject to psoriasis as in claim 4, the method comprising:
comparing a portion of the SEEK1 gene of the subject with the SEEK1 gene of SEQ ID No. 4; and
determining the presence of a nucleotide substitution, deletion or insertion at one or more positions of the SEEK 1 gene of the subject corresponding to positions 50049, 49405-49407, 49133, 49045, 49038, 49017, 48920, 48773, 47938, 39880, 35732, and 25534 of the SEEK1 gene of SEQ ID No. 4.

6. A method of diagnosing psoriasis in a human subject, or determining the susceptibility of a human subject to psoriasis as in claim 4, the method comprising:
comparing a portion of the SEEK1 gene of the subject with the SEEK1 gene SEQ ID No. 4; and
determining the presence of a nucleotide substitution, deletion or insertion at one or more positions of the SEEK1 gene of the subject corresponding to positions 50049, 49405-49407, 49133, 49045, 49038, 49017, 48920, 48773, 47938, and 39880 of the SEEK1 gene of SEQ ID No. 4.

7. A method of diagnosing psoriasis in a human subject, or determining the susceptibility of a human subject to psoriasis as in claim 4, the method comprising:
comparing a portion of the SEEK1 gene of the subject with the SEEK1 gene of SEQ ID No. 4; and
determining the presence of a nucleotide substitution, deletion or insertion at one or more positions of the SEEK1 gene of the subject corresponding to positions 50049, 49405-49407, 49133, 49045, 49038, 49017, 48920, 48773, and 47938 of the SEEK1 gene of SEQ ID No. 4.

8. A method of diagnosing psoriasis is a human subject, or determining the susceptibility of a human subject to psoriasis as in claim 4, the method comprising:
comparing a portion of the SEEK1 gene of the subject with the SEEK1 gene of SEQ ID No. 4; and
determining the presence of a nucleotide substitution, deletion or insertion at one or more positions of the SEEK1 gene of the subject corresponding to positions 49017, 48920, and 48773 of the SEEK1 gene of SEQ ID No. 4.

9. A method of diagnosing psoriasis in a human subject, or determining the susceptibility of a human subject to psoriasis, the method comprising:
comparing a portion of a SEEK1 gene of the subject with a SEEK1 gene of SEQ ID No. 4; and
determining the presence of a nucleotide substitution, deletion or insertion at one or more positions of the SEEK 1 gene of the subject corresponding to positions 50049, 49405-49407, 49133, 49045, 49038, 49017, 48920, 48773, 47938, 39880, 35732, and 25534 of the SEEK1 gene of SEQ ID No. 4.

10. A method of diagnosing psoriasis in a human subject, or determining the susceptibility of a human subject to psoriasis as in claim 9, wherein the nucleotide substitution, deletion or insertion is at one or more positions of the SEEK1 gene of the subject corresponding to positions 50049, 49405-49407, 49133, 49045, 49038, 49017, 48920, 48773, 47938, and 39880 of the SEEK1 gene of SEQ ID No. 4.

11. A method of diagnosing psoriasis in a human subject, or determining the susceptibility of a human subject to psoriasis as in claim 10, wherein the nucleotide substitution, deletion or insertion is at one or more positions of the SEEK1 gene of the subject corresponding to positions 50049, 49405-49407, 49133, 49045, 49038, 49017, 48920, 48773, and 47938 of the SEEK1 gene of SEQ ID No. 4.

12. A method of diagnosing psoriasis is a human subject, or determining the susceptibility of a human subject to psoriasis as in claim 11, wherein the nucleotide substitution, deletion or insertion is at one or more positions of the SEEK1 gene of the subject corresponding to positions 49017, 48920, and 48773 of the SEEK1 gene of SEQ ID No. 4.

13. A kit for use in a method of claim 9, comprising one or more agents for detecting a nucleotide substitution, deletion or insertion in a SEEK1 gene of a human subject as positions corresponding to one or more of positions 50049, 49405-49407, 49133, 49045, 49038, 49017, 48920, 48773, 47938, 39880, 35732, and 25534 of the SEEK1 gene of SEQ ID No. 4, in a portion of the subject's SEEK1 gene.

14. A kit as in claim 13, wherein the one or more agents are for detecting a nucleotide substitution, deletion or insertion in a SEEK1 gene of a human subject as positions corresponding to one or more of positions 50049, 49405-49407, 49133, 49045, 49038, 49017, 48920, 48773, 47938, and 39880 of the SEEK1 gene of SEQ ID No. 4, in a portion of the subject's SEEK1 gene.

15. A kit for use in a method of claim 14, wherein the one or more agents are for detecting a nucleotide substitution, deletion or insertion in a SEEK1 gene of a human subject as positions corresponding to one or more of positions 50049, 49405-49407, 49133, 49045, 49038, 49017, 48920, 48773, and 47938 of the SEEK1 gene of SEQ ID No. 4, in a portion of the subject's SEEK1 gene.

16. A kit for use in a method of claim 15, wherein the one or more agents are for detecting a nucleotide substitution, deletion or insertion in a SEEK1 gene of a human subject as positions corresponding to one or more of positions 49017, 48920, and 48773 of the SEEK1 gene of SEQ ID No. 4, in a portion of the subject's SEEK1 gene.


SEQUENCE LISTING (109 sequences)

SEQ ID NO: 1 - DNA, 861 bp, Homo sapiens
  CDS (275)..(730)
caggaaatcgagactcatgactcccagagaggatggcatctagaagtagacgatcaagggtggaatctacagtccatgggccctgacttcttgccttcgtctcaaatagactctgcagccagccatctatgcagcgccccagtggctttgaaatgcaacagaaaccatcacccccggaccgtgggctccatgccagtgggcaaagcacagcctgggaagaattggtttgcagccaggcagtcctccatccagtcttgactttggcacttgtgatatgacttgcacagaccaaaaaagtcactctcaaagagctctcggcacacagaccccagctttacaaggaccccagctccttaacacagatcccagctccaaggaaactcgtcccccccacgttaatcctgaccgactttgccacatggagccagcaaaccatttctggcatgcaggggacctccaagcaatgatatccaaggaattccatctggcagccacccaggatgactgcagaaaaggaaggacacaggaggatatcctggttccctcttcccacccagagctgtttgcatcagtcctgccaatggctccggaagaagctgccaggctccagcaacctcagccccttcctcctccctcaggaatccacctatccgcctctaggaccttggctccaactctattgtactcgtctcctccctcccattctccttttggtctcagctccttgatctaagcctcccagagagacccctagaatgtttccctcaaggacctttctgcctggaagtctgttagcctttcagaagtaacatgtccaaaataaaatttgattcctcccaggttgttccctgcctggtccgc

SEQ ID NO: 2 - protein, 152 aa, Homo sapiens
MTCTDQKSHSQRALGTQTPALQGPQLLNTDPSSKETRPPHVNPDRLCHMEPANHFWHAGDLQAMISKEFHLAATQDDCRKGRTQEDILVPSSHPELFASVLPMAPEEAARLQQPQPLPPPSGIHLSASRTLAPTLLYSSPPSHSPFGLSSLI

SEQ ID NO: 3 - DNA, 861 bp, Homo sapiens
gcggaccaggcagggaacaacctgggaggaatcaaattttattttggacatgttacttctgaaaggctaacagacttccaggcagaaaggtccttgagggaaacattctaggggtctctctgggaggcttagatcaaggagctgagaccaaaaggagaatgggagggaggagacgagtacaatagagttggagccaaggtcctagaggcggataggtggattcctgagggaggaggaaggggctgaggttgctggagcctggcagcttcttccggagccattggcaggactgatgcaaacagctctgggtgggaagagggaaccaggatatcctcctgtgtccttccttttctgcagtcatcctgggtggctgccagatggaattccttggatatcattgcttggaggtcccctgcatgccagaaatggtttgctggctccatgtggcaaagtcggtcaggattaacgtgggggggacgagtttccttggagctgggatctgtgttaaggagctggggtccttgtaaagctggggtctgtgtgccgagagctctttgagagtgacttttttggtctgtgcaagtcatatcacaagtgccaaagtcaagactggatggaggactgcctggctgcaaaccaattcttcccaggctgtgctttgcccactggcatggagcccacggtccgggggtgatggtttctgttgcatttcaaagccactggggcgctgcatagatggctggctgcagagtctatttgagacgaaggcaagaagtcagggcccatggactgtagattccacccttgatcgtctacttctagatgccatcctctctgggagtcatgagtctcgatttcctg

SEQ ID NO: 4 - DNA, 55050 bp, Homo sapiens
  misc_feature (13351)..(13351): n is a or t or g or c
gatcctgccttttcacaccaccacctggctctgctgacacatctagtcacagacccctgtgatgctgttactcagcaagtccaaagcttgcccttgtcacccccttcccacctgcacagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtgagtgccatgtctctctgcgggctccatctctttcccctgtcaccacctcgctttccctagctctggctcctccaactgctctagggctgttggctttggacagaatgtccaagcagtcaggcctgtctcagctcattctctaatgtcctcctctaactgctctagggctgttggctttggatagaatgtccaagcagagtcaggcccgtctctcagctcattgtctaatgtcattctcctttctgtcattcactggcaggtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcttgggacacagtagatagacacacttatcttggtttgtccttcagttactgaggtggggatgggaatatccaatgctcatacccaagtgaccctgaaactaaggtgccatttacactccttaaggtcacacaacatcagagggagagctgggattgcagccaagtttatttgtacagggccctgtgataggctagttcccaaaagcctgtgatgcaagaacttttgcccatagactcagtcaccatgtagctgttacctgttcagagctggctttttgctttcccaccctactctggaattcttaaatggctttatacttagaaatcatcttatttctgttgaacctagatcaccccaaccagaaacttctattaatactttgtgctttcttgataccagggtctatttggtttccacttaaggtttttgcatactctgcccataagtgactcattagttactcaagttttattcctggctctgccactagttcattaggggtctttgccccagagtcatttcttccatgtaaaaaaacttgggctcattaaatctaggtaggaaagggcggatgtggcaggttttaatagaacaggtcaagataaggctttatttctatagaaatgatgctttgacaatagtttggcttggtgtaaggctcacaaaagaaaatcacatgtaccatgtgtgggttaagcggtttgattcacactgaaccaggccagcccagttgccctctgctgtgtccacccgtggagtggagctgtgtcacagccatcacactggtaaactgctgtagctggtttaccaggctttctcttgccctgacagtacaggtgaagcctgtaaataaatcttctgctatctttgtgaacttaaccaaatcccagttaccttatttaaatggcaatagatctgttttcccttaaactagaaaccttaattacctgtattcctacctccagctcaacccatatatttgcacctttccagtaagcaggtttgtatttccatctctccccttcccctaagattctgaattagttctccagaccttgccaagcacattctccctggaaagcaaggaatatcacagacccacaagaagagtaaatgcccaggagtgaatgaagcggcttgtccttgacttggaaataaaagcaaagctgtgaaaagccaggtcgctacgattttgttggcagcaggactagccacagagtagggaagttttggggccaggccctaggttttcccagaatgccttgggtgatgccaccaagaaccttaagaactccctcttacattttccatcggttgcaggcatggcttttgcaactgataggtgctctgactacagatattctggtgtccatggcaacatggccttatggcttgtaatagtgggaacttccagctcactggcaatttcctggaggtggcaatatctttggcaggaaaccatctcttctgtcttggccaccagggtctgctccaggccctgaatgagtccacatcaatccatccttcttccctgtagtcattccctcttggggagggaagggaaggggggtatttatcagacctatttgctcgctgctgtgtctactcatgggcaggtgtgtccgatggtgccttggcggtggaccttaggtctgaatgccagcccggcaccctgggcatcggaagtcggcctccctggcagcctggatgctgcagccaacacaggccacatggaaccagacgtcacagccatcacactgaacccaggccactgtctcttcctggggcaggcagcaacaaggagctgcacagggctccccgcccccaactgcagggggagccatgggagcgctcactgggtacttccgaggacggccacgtcgatttctgaaggaagtgacacagacacaagggtcaccggaaacctgtaggagaaggcctagaccttgccaccctatggctggtcatagggtgaggccacagaggcacagtggtctgagaactaaggtgagcctggactctgtccttaaaaacacatagggtgggcctaaaaacctctagtgtgagcctgaaagttccccatccttaggcactagaagaatggggttaaagccatatgaatgtaaaaagcaggacatgctcactccggttgttttataagcactgccccaccccacctctcatgttctcctgcacatatttttcttttcctatcagagctaacccaggtcttgccattccatctattccacctcccgtgcccatccccccaggcagggggacctcctcacagcctagggatacaaggaatcgaaaagcagtaaaacactgagagggccaagtgaggactccacttacccagtgggagtcagtggggctttgtctacacggagtttcttcctgggctccataaggaccggtggcgggttctcaggaggctcactctcatcatccagttccgccaacactcggtgaacagatttcctccgattgcgttggggtggagcagaaggcgtggtccccacttccccaggtggggcgggaacaggcaggctctttggtccaccccaactaggggagaaggtgaggggctggggccggagcttgctgaggctgcctatggagtttaggatcagtgtggccttgggggcaggggagaaggtgctgagaggccgctgtggagccccctgggagggcagcataggccggaaaccagccccaacccgggcttctcccctagaccgtgggatggtaatggcagcaaagtcctgaggcttgactcgtacttgttggaacatgaagtagaactccgaggggctggttcctgggggcccttcagggccaaaggtcaggaggtctccatcactcaattccagcctgtgacctcttgggagtcggacattattgaccaaagtacctgttgatggtggggcaccaggcaaatatggaggacaaaaacagaaatgaccagagtcaggagtggagaaatctgtatgatttctggggaagaatatgaaactgtagtcagattctccctcacatcctaagcccctcagcaggtgacaggtaccaggcagtacactccttgtcagctaggaacaagtctgtgtttcttgtgctttcctacaaaggatccctgccctcactgtcctaaggtccaattttctttctttcttctttttttttctttcttttttttttttttgagatggagttttgctctattgcccaagctggagtgcagtagtgcaatctcagctcactgcactccgcctcccaggttcaagcaattctcctgcctcagcctcctgagtagctgggattacaggcatgtgccaccacgtctggctaatttttttatttttagcagagacagggttttaccatgttggccaggctggtctcatactcctgacctcctgatctgcccgcctcagcctcccaaagtgctgggattacaggcgtgagccaccgtgccccgccctaaggtccagttttcaaacatgctcactctcccccagctccagctcgttcagatcctagctctaccacttaccagttatgtgaccctgggcaagcttttggtttgtttgtttgtttgagatggagtctcgctctgtcgcccaagctggagtgcagtggtgcgatctcagctcactgcaacctccgcctcccgggttcaagtgattctgctgcttcagcctcctgagtagctggggttacaggtgcacgccaccacacctggctaatttttgtatttttttttcagcacagatggggtttcaacatgttggtcaggatggtctcgaactcctgacctcgtgatctgcccatctcggcctcccaaagtgctgggattacaggcgtgagccaccgcgcccaggctttttttaaaaaattaattaattttttggtagagacaggttttcaccatgttgcccaggctggtctcgaactcctgagctcaagtgatccaccctgccttagcctcccaaagtgctggattataggtgtaaaccaccatgcccagccctggacaagttccttaacctctttgtgcctcattttcctttactgtaaatgaggataatattagtaccacctacattcaatacacttaaaatagggcctggctcataacagtaagcactagatgaatgttggctactatagtctaataaccataacactcctacagctgactttaccaagaggcagtggcaaaactcaaatgtgaatctaggctttctgaggctaactgcctcaagtcacggagaaagacatgcccagattattaaaaacattattcaacatagaactgccctaacccaggaacagagtgcctcagcaaagctggaaactgaatcaagtctaaacttggcctctagtggcctcagaacatccccactgtgatatctttctgatgatatcccaggtcctgtatgagctaaacgggaccaaaaagggtcagtctggcagagagcttctgactgtgcacacttctggtgggaagatgaaggtctggaccacgactatccatccaatctgatgggagtatgggaggcaggcagcaaggtcttgtggagaccttactcattccagcctggcgcttcaaccacccaggggcaaagctgccctgcttaatgctcaccttggctgctgtggtcttccaggctgaccctccagtcatcaccccggggctcggcatgcagttcggcgtggatcccagagatgaggccaggctcctgctggggccgcagggccacatcacacaggtcggccctgtggcccaagcgataggtgcagccagccccggcgggggggtggaaggtgtagagatcaccgcccctgccgccccctatgcgcagcagttggaagcagggcagcatgggcggtgccccctctgcaccacctcttccacttcaggcatccatttcctttcccactcctgggccacgcaccgctggcttaagttcgcttcccagtctgatgcaaacttgagttgtgctgtatgcggtttcaactttgagcacactacctgaatagcccaaattcagagtgcaagcagcctaggactcaatgcaaaattggttaagccgacttgcaaatgaaaaattccaggaaggtctccgtgcaatgcaaactcaggaacacgtcctatggcacctcacttgagctggatcctcagatcacaaaatatttggggagcctccaatctgcaaggcaaggatactttaggcagctcctgtgtgtgtcggggtggcggggcgcggggaaggaggtcctgttatacacacgtctacgtgcaatacgaacacatcacctctgtgtaattcggagaagagcagcttcttcctgtcaaacttgcttatcctaatagagcactcctgcttgcctgcgccccgatcttctggccaatccctcactcataactggttgaacctccgagggcaatgaatccactgtcatcccccaaaagaaagtgcaaacgagtccttaagtactccaacaaattggacctaaacccctacttcccggcttctctaggtgggataaaaacaggaaggagctccccgcgatcatgcaaggcatacccggccttttcccactccgccccagccccgtttcggatctacccgcgcggcgcagtctccgagtagcctcctcttcaagcagtgccagcctgtgcggcggatcccgggaccccttgggctcctcggcaggagccgacgttgctgcatctgtttgacagccaagaagccggggccaggaggggcgcggcctctgcgcgcgggcagcgcctacccctccttcggaattcccgggcccgaacttcgcgccgagcgagcccgcccccgtgccgttcccgattggccagctcatttgttctcttccgcgccattggacagcgccccaagatctcccgccttccactttttctgattagttcacaagttttcccgggttgccgcagtgaggaggagccgcctgggcgcggagatgctccgacgcatttagtgggcccaaatttagacgccactgcgcctgcgtacacctttccccctcctcccaacagagtgaaataggactttagggcgcccgccttgcgcaggcgcggtcaggggcagaggcggggccgggagcgaggcgtagggggtgtggccaaagcgcagtagggggctctcgcggttggtaagggacgttcgggaagagtcggttgggggcgggaaggggctgagcgggggtcctgggagggttcaaggggtccaaaggggaagggggtacaggggccaggtgaagggacagatgaagagatcagaacagccttagagctcacgggctaactgggtgaagcctctggtatctaggggagcagtggtgggagagtagagccggctagcgtacagggggcgagggcaggctattgagacatgggggtgagggtcaggaccagaataattccttcagaaaaaggtagattaggttgtaaaaagggacgggagtagggatcccagattcgggtaaaaaatagattaatgagggtgggagggtgggaggaagatcttcaagtgcccactatgtgttaggactcgagaaagaggaggcgggcagcaggcatggaagctaaggggatagtagagagaagctgagagaacagccgcaggaaagatttgaataagatgtcaggacgctggcttttctgcctggggatctaggcggcctggattcccaaggtcttggggagggtctagccctgcatgtggccacattcagctggggccaggccttgggccagcactttaacagggaaggacccaagagtcatggcctgctggtgtctggatgggcttccctcaggccttgctgagccatggagagaactctggagatggcgctcaagacccctgcactgtgtacctcctttctcacctctggccaggagcagcagggaccatagaaacttaaggaggagggtaaaggcactgcccagaatcctgaaagtatgagtaaagcctcagagcccctctcagtctgtctctttcagtaaattctctccacttgatagttagttgcttgggttccatgaggaaccaggttgagacaagcaagtgaatttggggtatctcctactttggggaagtaggaaaagagctaagcatcgggtggacctggctataattcaaggattatgactttgttattgtaggggctttctctttggaaggtggcctgaaaattgaattggagtgtctttgtttctcctcttgtccaggggaacatagatggctgaagacagaatctagagccttcaaataatgtggagatgtttccaccttcaggtcagtgggaccagacaaggggagtggtggttgtctctgcctgggaaactgaccatctttgtttttgtaattcttcaggttccactgggctgattcccccctcccactttcaagctcggcccctttcaactctgccaagaatggctcccacctggctctcagacattcccctggtccaacccccaggccatcaagatgtctcagagaggcggctagacacccagagacctcaagtgaccatgtgggaacgggatgtttccagtgacaggcaggagccagggcggagaggcaggtagggatccatccacgccgttttctcaggcttgcttgctagtgacccttcctcactggaataaactccttaccctatttcggccctagtttccagaacgtactcatttttatgtaagcaattagttcctacagtgcataaataataaagcagtgtgctaagtgctagggatgaggataacaatagtgaataagacaaatcctgacttaaatttgtaattaataaggaatctaaaatcctaagaggggagatagattgaccaccagctgatggtaaagcatggtatgaggcaagatgaagaagtgcaaactagaggtagagacccagcactgtggcagtgtgggttcattctgatggaagaaagcctttacagaggacatgctgtgggagctggccatgctgtcgtattatctgtaaacattcttattcttgtttcatgtgtcttttcccagatgtactgggatatcattctgccattttgctccagaataggattacattagaagaagcagcatgatgaagaaggaatactagactgggtgtcaggagacagagactctatttctagtttggctaccagctagaggacatttggcaagtcacttaatttccctagcctacagatcgctgctactaaagaaagaaagaaagagcgagccagatatggcggctcacacctataatcctagcactttgggagctgaggcaggaggatcacttgagcccaggagttcatgaccagcgtgggcaacaaagcgagactccgtctccacagaaaataattagctggctgtggtggcatgcatttgtagtcctagctactcaggaggctaaggtggtaggaccccttgagctcagaagttgaagactgcagtgacctatgatccagccactgcattgaggcctgggtgacacagtgataccctgtctctaaaaacgacaacaacaacaatctcttatagtcctgggtctcagagagctgcctcaggagccatgttccaagctggattaaacttcacgtgacattggtagacgatttctctaaaggctggcactgtgttatttatgtactgttctctcagactcctcataaagtatacctaacactcaataaatgcctttttttttttttttttttttgagatggagtctcactctgtcgcccaggctggagtgcactggggagatcttggctcactgcaagctccgcctcccaggttgacgccattctcctgcctcagcctcccaagcagctgggactacaggcacctgccaccacgcccagctagttttttatatttttagtagagatggggtttcaccgtgttaaccaggatgatctcgatctcctgacctcgtgatccgcccgcctcggcctcccaaagtgctgggattacaggcatgagccaccgcgcccagccaataaatgccttttaactagcacctggcctcaccatattgatactggaagcttacgacctctctatgcccattcctccctcaaacttcttgcccttaaattagaattgagaagtccctgtgtgttcttccaacctttccacttaaaatgtgtggcctaaaaataaaaaaataaataagacaaaaaaaccaccaaaaaacaaaaagaatgtgtggcctaatgatatacacatttgatgttgaatcctcctgtatgtagctctctctagggagatcatgttccacgatctcagccagactttaggtcctgtgagtccaggcactggactcaacatgctcaatagggctttgatgaatgatgatgatgtcaatgcagacatccccataccccagcttcagcacccccttcacctccccacacggaagcagaggggtcctcttttccttctcctggctatgtttatgccctcaactatccttccagcactggagacaagtctcacctgcactaacctgtctttgaaggtcctgggggctggaggggtcacaggccctgagccagcaggctgaggtgatcgttcggcagctgcaagagctgcggcggctggaggaggaggtccggctcctgcgggagacctcgctgcagcagaagatgaggctagaggcccaggccatggagctagaggctctggcacgggcggagaaggccggccgagctgaggctgagggcctgcgtgctgctttggctggggctgaggttgtccggaagaacttggaagaggggaggcagcgggagctggaagaggttcagaggctgcaccaagagcaggtgaatgcaggggtagaaaggattcaaattcataacggagagctgggcagtagcttccaagcaaagaacaggtattgcagaaaagaccctccatgagtagtgagtagtagagtgatgagacctttggtgaaaataaacacacatgggctagaaagatggagaatttgggtacttttatcttaattcaggttgcacttttcccccaagactcaggtggcctcccaccacttgaagccctgcttccctttctagtaggaaatagtttgcctccctactttactccaggtaccaattcatcaggggtattgtggagggcagtgaggaaggtgggtataagggggctatggtggactgggagagagagattattcagtcctcaaactcagtacttactctgccatctctaagactcagtgaccaaattagcctgagtcctgccttcgtggatggaactgacagcctcgtgggggatatacacatccacatttaatttaatcataattaattgcaatcaggaggaatgccttgaaggagaaggactagaaagcactaacttagtctggggttcagggaaggccactgtaaccagttgacatgtcaaggaaagaaaccatagccctgggcgcagtggctcacgcctgtaatcccagcattttgggaggccgaagcaggcagatcacaaggtcaggagttcaagaccagcctggccaacatggcgaaaccctgtctctactaaaaatacaaaaattcaccaggcatggtggtgcgcacctgtaatcccagctacttgggaggccgaggcaggagaatcacttgaacctgggcagtggaggttgcagtgagccgagactgtgccattgcactccagcctgggcgacagagcaagactctgtctcaaaaaataaaaaaggaactgggagaaaacaagggagaattcctttataaccttgtagtgggcaaggcctttctacctgtgagtcaaaatccaaaatctagaagccataaaggaaaaaattgatccattgactttataacatgaacattaggaatagccaaaaagaaaaaaaaaagctatatttatagctcagatcacaagaaaagggtaatatccctaatataaaatgtgtgcctagaaattggtaagggaaagaccagcaatccaatcagaaaatggacaaaggagatttatgaaagaaacttagaaacaagaagctaggccaggcacagtggctcatgcctgtaatcccagcactttgggaggccgaggtgggcggatcacttgaggccaggagttaaagaccaacctggccaacatgaagaactctacaaaaaaatacaaaaattagccgggtatagtcgtgggcgcctgtaatcccccagcttcttgggaggctgaggcaggagaattgcttgaacctgggagacagaggttacagtgagctgacatcacactccagcctgggcagcagagcgagactaaaaaaacaacaagctaccgtttgtgctgaataggagttggccagtgaagaggcgtgtgaagtccagtggtagctggaagacacttggtgggacaacaggtgaaggcggggacaggaggccagaaggctggggcacagagatgaggggcactgagtgtgctgcagagcccaggacccagggcacaaggctttggccacttcagaacttgctactttcccataagagcaatgagcaggctgggcacagtggctcatacctgtaatcctagcactttgggaggccaaggtggaaggatcatttgagcccaggagtttgagaccagcctgggcaacaaagcgagacccccatctctattttatggaagaaattagggctgggcatggttgctcacatgtgtaatcctagcactttgggaagctgaggcgggtggatcacttgaggtcaggagttcgagaccagcctggccaacatggtaaaacctcatctctactaaaaatacaaaaattagctgggcgtggtggctcatgcctgtaatctcagctactcgggagggtgaggcaggagaatcgcttgaacctgggaggcagcgtttgcagtgagctgagatcgtgccattgcactccagtctaggcaacaaagtgaaactccatctccaaaaaaacaaacaaaaaaaaattgttttttcaagtaataagcaaccgttgaaaggttgtttttttttttttttagatggagtctcgctctgtcgcccaggctggagtgcagtggcgcgatcttggctcactgcaagctccgcctcccgggttcatgtcattcttccgagtagctgggactacaggcgcccgccaccacgcccagctaattttttgtattttaatagagacggggtttcaccgtgttagccaggatgatctcgatctcctgaccttgtgatccacccacctcgngcctcccaaagtgctggaattacaggaatgagccactgcgcccggcctgntgttgaaaggttttaagcagggaaataacatgattagatttgtattttatgtctaaaaaattttgtcatttatgtcccccaaattaattttattgttgtatggagacagggctagaggaggcagaccaggaagcagggtgggcactttgccctcctttccagtccatcccatgactcttggtggctctgacacccctgcaaccctttgaggtgccatgagcaaaagacacaaaattcctcctttcctggagctttccttccagtgtggtccgacagatagtaacacatacacataagcaagatatggtcagtgctaagtgctcaggaggacgtgaacagctgatggggcagagtagggtggggagggacggtattagagggcccagtgaagccaccctgaggaggggctattgcctggggtctgtggagcaaggaggggccgctgtctggttctcagcagactccccgtggccggagcggggagcagtgggagagcctccagggtgagctcaggaggtaggcagaggccgggtcccctggcctgcaggtgtggagagacacctgggttttgttgtgaatgctgtgagaagccactgagggtttgtaaagactagttaggagatggtcgctgttgcccaggcaaaagatgagggttggtggcagtggagacggagacagagaggtgaagatatgttttgggggagatcggacaagaactcctgatgggttgtggggcagctgcggagagtgagttgccagctctccatttgctgtgcacagttggctgattggttgggtcattctctaaggtcacagaaagtgggagtgaagggaacaaggaaggcctccgtgtggggtcgagcctctgctgagccccctcttctttccgcagctgtcctctttgacacaggctcacgaggaggctctttccagtttgaccagcaaggctgagggcttggagaagtctctgagtagtctggaaaccagaagagcaggggaagccaaggagctggccgaggctcagagggaggccgagctgcttcggaagcagctgaggtaggtgggcggacgccgacgggagcccagcaattagtgatgtggtggatctgcagggcgccccactgatggctgtcccattcccaccccaaccctagcaagacccaggaagacttggaggctcaggtgaccctggttgagaatctaagaaaatatgttggggaacaagtcccttctgaggtccacagccagacatgggaactggagcgacagaagcttctggaaaccatgcaggtgagggtgcaggaatgtatctgtgtgcagacttagggatcaggttgggaggcaagcgtggcccttggaggagcgtgtagagcacagcctccgggagagaaggtggtacctaaggcggcatggaggccctacagaggggctgctttcctctgcccgcagcacttgcaggaggaccgggacagcctgcatgccaccgcggagctgctgcaggtgcgggtgcagagcctcacacacatcctcgccctgcaggaggaggagctgaccaggaaggtacagcccaacccccagacccctcaccctcagccgcatcctgcatctactgtcccctgcctccctccctgtgggcaggaggggtcaatgtgccccagaacctgcttagatctccttcctgtgaactcctcttgctgtagctcatgttgcccaggcaggacagaggagaaacaaagatgccacctccttcctctcctcccccaggagcccacgcttttctcccactccttctccctcaggttcaaccttcagattccctggagcctgagtttaccaggaagtgccagtccctgctgaaccgctggcgggagaaggtgtttgccctcatggtgcagctaaaggcccaggagctggaacacagtgactctgttaagcagctgaagggacaggtcactgcactctcttttctcccggtattccctcccagcaccttgctccttccatgaaggtggcatccattcaaccagtgtttattgagtggttgccacatgctgggcacacagccctgaacaaaactaaaatgtggagcttgcattctagaacagagacacagaacacgcaagtaaacagataatgttgggtaattatatgtgcgatagaaagattgaagccgggtgcagtggctcacacctataatgcgatcactttggtctcgaactcctgacctcaggtgattcacctgcctcagcctcccaaagtgatgggattacaggtgtgagccaccgtgcccagtcaagtaatgccaacagtttgggagaccgaggcaggtggatcactggaggtcaggagttcgagaccagcctgggcaacatgtgaaatcccgtctctactaaaaatacaaaaaattagccgggcatagtggctcattcctgtagtcccagctactctggaggatgaggtgggaggatcacctgaggctgggaggtcgaggcgaggccacagtgaactgtgatcccatcactgcactctagcctgggtgacaaagcgagatcttttctcaaaaaaaaagaaagtagtaagaaaaattcaaaagataatgtgacagagagactgtggggtgagtcagcctcaggtaggatgctcagagacagcctctctgaggaggtgacagcatctgaggagagtggcatggtcagttggtgggtcttgtggggtgggtcaagggctattcccatcttcgagtgggcacatggaatgtggaacatggaacactgggctcagattccatcctcagaacctaagcttctgtctccctgcgtggcattcattctttttcttttcttttctttttttttttttttgagaaggagtcttgttcttgtcacccaggctggagtgcagtggcctgatctcagctcactgcaacctccgcctcccaggttcaagtgattctcctgcctcagcctcccgagtagctgggattacaggcacatgccatcacgctcagctaatttttgtatttttagtagagacaggctttcaccatgttggccaggctgatcttgaacccctgacctcaagtgatccatctgcctcggcctcccaaagtgctgggattacaggtgtgagccaccgtgctgcgacccacccccgtcgcccgccctccctccccccagcccctgcatggcattcttacagagatctctgcacctgccactttgcttccagtgcccccctcatcttttagctctagagggccctgcccagctctctctcctcccccaggtggcctcactccaggaaaaagtgacatcccagagccaggagcaggccatcctgcagcgatccctgcaggacaaagccgcagaggtggaggtggagcgtatgggtgccaaggttggtgtcagcctactagagactcggggagggcaagggagcccctgttccggggctgcagccaggacttagggagggaccctgtcctttgctgcatcctccccagggcctgcagttggagctgagccgtgctcaggaggccaggcgttggtggcagcagcagacagcctcagccgaggagcagttgaggcttgtggtcaatgctgtcagcaggtatcagggatggaggggtgggtggagtagtgtttctgctacctcaggttcctgggcaccttgttgctgaggatcctcaggcaagaggggctggaaagtggccactggaggctacagggctgggcagatttagctctatcaatgttcctgtgttcgtttcttttcctggggaagccccttctgcattcatacctgattgcttgttatgaatttcccgttgcatgtttggctggaggtgaggccttgcttcctcctgcagttcagtctagtaatggcttgagctaaatagagcacccgggaggatcttcacttgcagtattgttcaaggatggagagtgtagacacttcatcttcctttttttttctaaaattttacgggcaatccgtttcactggagaaaaatttagtctatttatttatttattttgagacaaagtctcgctctgtcacccaggctggagtgcaatggcrcaatcttggcycactgcaacctcacctccctggttcaagtgattctcctgcctcagcctcccgagtagctggattacaggcatcctccaccagtgtcctccactacgcccggctaatttttgcatttttagtagggacggggtttcaccatgttggccaggctggtcttgaactcctgacctcaggtgatccacccacctcagcctcccaaagtgctggaattataggtgtgagccactgcacctggcctagtctatttatttaaagctgtatacttacttgcttattatatacttaacttgcttactattccatctaaaatgtaagccagttagtttccttctaaatcaattgccagcctttgtctctcctaccaacttcctagttgtttcattacctacaattgttgtatgaccttcagaaaaacctctaagaaaacagcaaagcttctttgtgctggtgatgacttcccctcagccttagacactgaggtacccaaggcaggtagttctttttttttttttttgagacagagtctcgcactgtcacccaggctggagtgcaatggcacgatctcagctcactgcaacctctgcctcccgggttcacacgattttcctgcctcagcctcctgagtagctgggattacaggtgcacaccaccacacccggctacttttttgtatttttagtagagacagggtttcactgtgttggccaggctggtctcaaacttctgacctcgtgatccgcccgcctcggcctcccaaagtgctgggattacaggcttgagccaccgtgcccggccggcaggtagttcttagcacagtctctggcttgtaaatgttgattgttatcgtgaggctcttcttgatgggttaatttagataaagataatttttggtttagcgaaattaagatgcaggatgagtccttgcccaccacttccttctcttgggtttgtaccttagggactaatttagctttaaaaattatgaaaaatttcaaacttgcacggaattaaactagttataatgacctaccacccaggttcaatccatcgcgggtgccccctacctccaggagaacagaaagatgcactgtggacagggtttgactttggtaccaggtgatcacagaaatggtctgtgtaatgatgcatttgccgaaagtcctccaggcttaaagcagtccaaactctgattgttgctgggtttattagattgtctctaggtaattggagactttaataagagctgtggtaggtgttggaagcatctactggaacaatttccagatcaaagtgaactttgctgtgctgctggggatgcagctgcaagccttcatcatcatgtgtttttctgtgggtgcagacctggactctcctgaggaaaccccagcccggccccagacactccttggcctcctcctggggcctgtttaagctgctcagttttcatgagccaggttggtcctacttctggcacagccagctggtaaagcatgtggacctgcccgtcactggtgctagatcgacactcctgggcttggagaggataactttgttttcttttgtttttttgagatggagtctcgctctgtcacccagtcttgagggcaggggtgcgatcttggctcactggaacctccacctcctaggttcaagtgattctcgtgcctcagcctctggagtagctgggattacaggcatgagccaccatgcccggctaatttttgtgtttttaagtagagagagtttcaccrtgttggccaggctagtctccaattcctgacttcaggtgatccgcccgcctcggcctcccaaagtgctgggattacaggcaggagccactgcccctgaccagagaggataactttactctttgatacacgatagtgagcaaaacacagttgtgagaaataagcttaacaggttgcttaaaaagatagtcatttaatgcattcttggggcaagggtcctttagataattgacggaagctgtgcgttctgtacttgtataatgggacaggattagagggagttgtctatacaaggcacagcaagtcctttgggaatgaggggaggcatggaggatcagtgacttgtgccctctccagctctcagatctggctcgagaccaccatggctaaggtggaaggggctgccgcccagcttcccagcctcaacaaccgactcagctatgctgtccgcaaggtccacaccattcggggtgcgtaggacaactgcgagccacgtcctgcccccaccccaccagctcggactttcttcttcctgacccagctctctctgatcccacatccattcaccttcctcctttcaccagtccttgcatctctttttcccttactccctgtccccactttctcccatgcaaacttcatctctttttctccctgctttttccctcccaggcctgattgctcgaaagcttgcccttgctcagctgcgccaggagaggtgaagtttgggcactttgaggtggatggggctttagggcattggctgctgggacccccaaaaccatgaggactgaggtgggatgggggctttgggatcaggcagctgggtgatttctcctgactctttctcttccccgtctcagctgtcccctaccaccaccggtcacagatgtgagccttgagttgcagcagctgcgggaagaacggaaccgcctggatgcagaactgcagctgagtgcccgcctcatccagcaggaggtgggccgggctcgggagcaaggtacacctggttgccagagggtggagaggatgaggaaaaacccagtgtctagggtgctgggagaggcctgacccagcaccccctccttttaggggaggcagagcggcagcagctgagcaaggtggcccagcagctggagcaggagctgcagcagacccaggagtccctggctagcttggggctgcagctggaggtagcacgccagtgccagcaggagagcacagaggaggctgccagtctgcggcaggagctgacccagcagcaggaactctacgggcaaggtgtcgagagggaaatgggtgcttcccttggagggtggggtgggaactgcgaatcaaaggtcctgctgatatgccccgtctgcactttcaccccagccctgcaagaaaaggtggctgaagtggaaactcggctgcgggagcaactctcagacacagagaggaggctgaacgaggctcggagggagcatgccaaggccggtgagccttgccagggtggatagggccttccaggaagaaggaagtgttaagacataaggttattattttcccctcaaagtgtgttcaaagcttcattacaggaagtaatgaaggtatccaggagtagcacagatgaattatcacatcgtgaacacacccatgtagccagcaccagattaagaaacagcatatggccggtcgcggtggcttatgcctgtaatccaagcactttgggaggccgaggtgggtgtatcacctgaggtcaggagtttgaggccagcctggacaacatggcgaaaccctgtctcgactaaaaatacaaaaattagctaggcctggtggtgggcacctgtacmccaagcttacttgtgaggctgatgtgggaagaytacatgaacccgggaggtcgaggctgcagtgagccaagattgtgccactgcactcaagcctgggtgatagagaaagaccatgtgtcaaaaaaagaattgtgtaatgaatgtatcttctctaactaaatatagcagttaacatttgccacatttggtctcttatctatatacacacatatttgtacatcttttgaatcactttaagttgtaatcatttaatgttttgttgttgttgttgtttgagacagagtcttcctctgtcaccagctggagtgcagtggcatgatcttggctcactgtgacctctgcctcccgggttcaagccattctcctggctcagcctcccaagtagctgggattacaggcgcccaccaccatgcccagctagtttttgtatttkcagtagagacgggattacaccatgttggccaggatggtctcgagctcctgacctcgtgatccgcccgcyttggcatcccaaagtgctgggattataggcgtgagccaccacgcctaagtaagttgtaaacataagttgttcagccgcatctcccaaagccagtaaattctcctatatagctgcaatcatcacactttaagacagtgaacactaattgcacaaaatctaacccagttcatgttcagatttcccctgaggaactccaggatggttcagggatgaggaagatacttaggttcagattcccaggctcctagagcatcagccaacccctccaactgtacagaagagacagatccacagagcagaacagcctccccaagccacagagttggtgacccagcgtttgttcctgtcttcatggtgcctggctgcctctggcctgactcacacctgcctcctctgtgccttggcctctctgtagtggtctccttgcgccagattcagcgcagagccgcccaggaaaaggagcggagccaggaactcaggcgtctgcaggaggaggcccggaaggaggaggggcagcgactggcccggcgcttgcaggagctagagagggataagaacctcatgctggtaggagacaggagggcagacaggcagacactagggcccatcctgggctggttcctgggctagaggtgtggaaagaggatggtgagggaggctctatccgggctaggtttaaccctctccttcccaggccaccttgcagcaggaaggtctcctctcccgttacaagcagcagcgactgttgacagttcttccttccctactggataagaagaaatctgtggtgtccagccccaggcctccagagtgttcggcatctgcacctgtagcagcagcagtgcccaccagggagtccataaaaggtcttgggccaagcacaaagggacaagggacaaatgcgcgcacttcaggaatctcctcttcagactctcgcatgatgagtgttgttctctgcggtccttcgaggcccttagcctcttttagcgatgcccagcttggaccaaagagcctcctctctcccattcctcatttcctgtgccagccctgtttcctctgtaaccacgagcaccttcccttgtctggtgctcatctgctgtcttccttcccagggtccctctctgtcctgctcgatgacctgcaggacctgagtgaagccatttccaaagaggaagctgtttgtcaaggagacaaccttgacagatgctccagctgcaatccccagatgagcagctaagcagctgacagttggagggaaagccagcctgggggctgggaggatcctggagaagtgggtggggacagaccagcccttccccatcctggggttgccctgggggataccagctgagtctgaattctgctctaaataaagacgactacagaaggagccattgtttggaaatgttatttctgggaatctgtgggaattctccctgcagccatctctcttggagattgtgagaagtggtcccacccatagctctctctatgggtggcaccttttcaaatcctttttctaggcagctttggaaatgtttgggaagctcctgcttttctttattctcaacttttgttttaaaactcttttcttctggaagatgataaatgctaggagtggggaagatgaaacattcactcgttcaacaggaatgtcaatgccaggcactgtattagcctgggggccacttggaccctgagagcctacatcctagtgggagagmgagcgagcagatgggacctaaacagatgaacgagatcgtttcaaataagtgcttgaagagaaagtacagtacgatgatatgatagaaactgactctggggagaggcctctgagcaggggaccttgtgcagaggcctgcctgaagtgggctcctgtggaggccaggaggtctctgtagagaatgctcaaggatgctgctgcgaggccgagatgtggccaagtttggattttactgtaagtcaatagaaagccattggagtacttctttaaaaacggggaagaaagccaggtgtggtggctcacgcctgtaatcctgacactttgggaggctgaggcacgcggatcacctgagctcaggagtttgagaccagcctgggcaacatggtgaaacttcgtctctactaaaatacaaaaaattagccaggtgtggtggtgtgagcctgtagtcccagctactcaggaggctgacacaggagaattgcttggacacgggaggtggaggttgcagtgagccgtgatcatgccactgcactccagcctgggtgacagagcgagactcagcctcaaaaaaagaagaagaattaaaaaaaaaaaaaaaaaacagggaagggggcccggtgttgtggctcccgcctgtaatcccagcacttttttttttttgagacagttttgctcttgttgccaggctggagtgcagtggcacaatctcagctcactgcaacctccacctcccaggttcaagcaattctcctgcctcagcctccggagtagctgaatcccagcacttttttgggaggccgaggtgagcggatcgcttgagcccaggagtttgagaccagccaacatagggagactccatctatataaaaaataaaaataaaaaacattggagggcatggtggcgtgcacccatggtcctagttactccagaggctgagttgggaggattgtttgagcccaagaggttgaggctgcaatgagctgtgatcgcaccactgcaccccagcctaagagagcgagaacctgtcctcactccccaaacaaagggaagacgcaacatgatttggtgtacattttttttcctgagggacttactggatggtcccttccagagtgaggtacacatatccacgcactgtggtcagcgattgctcccggcacccagcgcagcagatgggtgggtctaaccaggtcacttccccaggagggcataattgagcagtttccgcatcaggtccacgtgggccagcagcatgcggcaggcagcggctgtggygcttcaggcccagggcgtccccatcagcgtacttggactgcagcagccccaggtaggcctcccacgtgttgcccacagtcttgctgcaggtgaagcagcgcacacaactcctgagctcaagtggtcctcctgtgtcagcttcccaaagtgctggcatgagccactacgcctggcctgatatatatatatatgttttttttttttttgagacggaatttcactcgttgcccaggctggagtgcaatggtgtgatctcagctcaccacaacctctgcctcctgggttcaagcgattctcctgcctcagcctcttgagtagctgggattaccaagaggcatgtgccaccatgtccggctaatatttttgtatttttagtagagacggggtttctccatgttggtcaggctggtcttgaactcccgacctcaggtgatctgcccgcttcggcctcccaaagtgctgggattacaggcgcgagccaccacaccaggcccgcgtgatgtatattttaagacctcttttgctggtggaggacaggctttgtgtgagggggagggataaacagtgggagcaagggggccaattagaagggtgttggggaggctcaggggagatggtggctcaggatgatgggctgggtttggacagggtgtggaggggcttgcaggtggatggtggaggagtgtaacgaaggtttctgcgtgagccctggagggaacagatgagatcacgccattgcataataaggtgttccttactgtggggtagcggaccaggcagggaacaacctgggaggaatcaaattttattttggacatgttacttctgaaaggctaacagacttccaggcagaaaggtccttgagggaaacattctaggggtctctctgggaggcttagatcaaggagctgagaccaaaaggagaatgggagggaggagacgagtacaatagagttggagccaaggtcctagaggcggataggtggattcctgagggaggaggaaggggctgaggttgctggagcctggcagcttcttccggagccattggcaggactgatgcaaacagctctgggtgggaagagggaaccaggatatcctcctgtgtccttccttttctgcagtcatcctgggtggctgccagatggaattccttggatatcattgcttggaggtcccctgcatgcctgaagaaggacatggtggagagcaggatgcctggatcccatgggggaagggaagtgcccaggaaagcacgaagccccagggggagctttcagtgcggggrtgagtggggaggctggggtagtagctgacactgtcccagctgcatcccaggtttgaaaggcacctcctcccccagcgcaggcatcctgcctcccaaccctgtaattacggtgcttcccaacgcccatcgygtggtttgctcccattctttggcttccaatagttgcaagggatgaaggtggacatctctgtgattacggagatgccaagtgggtattgactgctccagggtgtggatggagggtgtgaaaaccagggtggggtgacgcaggctctgggtcatgatagggagagcaggcagctgggtcctgggctggaggactaaaataagggacgccaccttcaggggtgacacatcagcccaggccttcccaacgggtttgaccagttctgttctgatggtattcctgtgccactgggctggyccctcctccactcctcccctataaagcctcttggggttcccaggcacccagactcagcccaccccagctttgggggccagtacatagccatgatcctcaactggaagctcctggggatcctggtcctttgcctgcacaccagaggtgaggtgggaacagaggcagggactgcagtttggggtgatgagggatactcaagatggcggaggtgaactggacgcatggggttggggacaggaattcaggggaygcagaaggtgcatctggctcaccagaaatggctttctggacacattgggtgggggacatggtgcagaaggtgcatttggctctcaccagaaatggtttgctggctccatgtggcaaagtcggtcaggattaacgtggggggggacgagtttccttggagctgggatctgtgttaaggagctggggtccttgtaaagctggggtctgtgtgcctgggggccaaggtgtaacccaccttgggttgcaggttggcctgaggacaaagctagtggggtaccccaaccaggggtggatggagcttatttggagaagtctggtcagtttaaagtgggtcaagtgaacggttcagatccatcgggggtaggggttcatgacattttaccatcagttaagtatttacaaacctaccgagagctctttgagagtgacttttttggtctgtttgtgggtcagttcaggctgcgtccrtccagacaggctcctcctcctggggctggggctgggtggggctggggagagaagccctcaccacctcttacctttctccttcctcctttacaggcatctcaggcagcgagggccacccctctcacccacccgcagaggaccgagaggaggcaggctccccaacattgcctcagggccccccagtccccggtgacccttggccaggggcaccccctctctttgaagatcctccgcctacccgccccagtcgtccctggagagacctgcctgaaactggagtctggccccctgaaccgcctagaacggatcctcctcaacctccccggcctgacgacccttggccggcaggaccccagcccccagaaaacccctggcctcctgcccctgaggtggacaaccgacctcaggaggagccagacctagacccaccccgggaagagtacagataatggagtcccctcagccgttctgttcccaggcatctccaggcacccacgccctctccaccctctgattccccgtgaattcttcccaatttagcctatctccttaaacctcttcctcattccctcggttttattctgaacccgtaaggtggtgttctcaatatttcctgtcccctcctgagatccatacttagtcctcacatcgcccgttttttcctctgacagcctaagcctactctcctacctcgcctccaggcctcggccccacctacctcccacccggtcttcctgcccgcgcgatcgctggggcagggctayggtactgtgttcccttctgccacctggtggccggcggcaggaactatcagtagacagctgctgcttccatgaaacggaaaaataaaaatcatgttttcttaaytctgaatctaggctgctgctttaactaacacttagggtctttttcatttatttttatttatttgtttttttctttttttgagacgaagtctcgctctgtcgcccaggctggagtacagtggcacgatctcggctcactgcaagctccgcctcccgggttcacgctattctgcctcagcctcccgagtagctgggactacaggcgcccgccaccacgccaggctaattttttgtattttttagtagaggcggggtttcaccgtgttagccagagtggtctcgatctcctgacctcgtgatctgcccgcctgggcctcccaaagtgctgggattacacgcgtgagccacagcgcccggcttctttcttctttttctttcttttttttttagatggagtctcactctatgcccaggctggagtgcaatggcacgatctcggctcactgcaacctccggctcccgggttcaagccattctcctgcctcagccttctgagtagctgggattacaggtgcgcaccaccatgcccggctaatttttgtattttagtagagatggggtttcaccatgttggccaggctggtctcgaactcctgacatcgtgatctccccgcctcggcctcccaaagggctgggattacaggcgtgagccaccgtgcccggccaacacttatgtttttgactattaggatgccctcttcacagtcctaaacttacggagacctggaagtaacttgagttcctatcttgcccatgtccagcatgtaaggctctggggcttagcaggaggagggttggaaatgtcactatgcaagtcacaataacattcaggcccacatttctccctttctgagaacactatattaaagaatgggaaggcaagtttcatctctgtttaatggcctatggcttggatacccctagtggtatatgcaaaccttcccaggggtgtgtcggcaggaccagttttaagggaatcagtttccagattaatatgtgccccccgctagaatgaatctcctgcttgtcctgggcctgaccagagtgcccttcccagagccgccaaaggtcaataggaaacaaatcaacctttcccatctcattaagagattcattttctttcttttcttttttttttttttttgagacgtattatctctctgtcgcccaggctggagtgcagtggcacgacagatatcagctcactgcaagcttcgcctcctggattcaagtgattctcctgcctcaacctcccgagtagctgggattacaggtgtgtgccaccacacccagataagttttctatttttagtagagatgggattttgccatgttggccaggctggtctcgaattcctgatctcatgggatctgcttgccttggcctcccaaagtgctaggattacaggtgtaaaccaccacgcctggccaagagatgcattttcaataagttacttttcatgtctttttgtgtgtttgtttgagacagggtctccatctgtcatccaggctggagtgcagtggcacgatcatggctcgtatagcttcaacctcctgggctcaagcaatcctcctatctcagcctctggcgtagctgagactacaggtgcaccacccctgactaattttttgtatttgtttagtttagttttgtttcgtttttagagatggggttttaccgtgttgcccgggcttgtctcaaactccagagctcaagtgatcggcccatcttggcctcccaaagtgctgagattacaggcacgagccaccgcgcctgaccaactttttatgtttaatccttgtgaatattcctagttttggttaactgcaataattgcaatacaaatagaataactgtttctaacacttgttcaagggcttgttcacgtattttttaaaaggatgctaacagatatgaaagttctatggcattatattcaatttgctacacttagagtgacgtgcagtctccgacagactgagcacaacaaattgtttttaattttaaaaactgacatggccaggcatggtggctcacgactgtaatcccagcatttgggaggctgaggtaggcagatcacttgaggtcagcaattcaagaccagcctggacaatggtgaaactctgtctctactaaaaatacaaaaaacttagctgggcatcccagctactcgggaagctagggcatgaggattgcttgaacctgggaggcagaggttgcagtgagccgagatcgcaccactgcactccagcctgggagacagagtgagactccatctcaaaaataataaataaataaataaatacataaatagtgatgtgatttttaacatgtatttgcaattccctgaaaagcctaccctttggaatgctattaaattattacaaatgttaaatgttgacttaaaaatgtgcaaggggctgggcgaggtggctcatgcctgtaataccagcacttcgggaggccgaatcgggtggattgcttgtggccaggagtttgagaccagcctaggcaacatggcaaaactgtctctacagaaaatttaagaaattagccagatgtggtggcccgcacctgtagtcccagctactcaggaggctgaggtgggaagattgcttgaccctgggaggttgaggctgtagtgagccaagatggcaccactgcactccagtctgggcaacagagtgagaccgtgtctcaaaacaatacaaatgtgcaagggacatagtttttcaaaatcctttaaagaggcaatcaggttagaaggacaggagctcagagatcccaatggtctactgtcaatcaagtatccgaccagggttagggatgaagaggggttaaaagaaactgaggttgcataaccttaaatttcaccacttagaacccagtttgcttatgtggtaactctcattaaaaactacatatgagaggccgggcgcggtggctcacgcctgtaatcccagcacttcgggaggccaaggcgggcgaatcacgaggtcaggagatcgagaccatcctggctaacatggtgaaacccagtctctactaaaaaatacaaaaaaaattagctgggcatggtggccggcgcctgtagtcccagctactcgggaggctgagacaggagaatggcgtgaacctgggaggtggagcttgcagtgagccgagattgtgccactgcactccagcctgggcgacagagcgagactccatctcaaaaaaacaaacaaataaaaaaccaaaaaactacacatgagatcaggcgtggtggctcacacctgtaatcctagcactttgggaggctgaggcgggtggattacctgaggtcaggagttcgagaccagcctcaccaacatggtgaatccctgtctctactaaaaatacaaaaaaattagctgggcatggtggcgggcgcctgtaatcccagcttctcaggaggctgaggcaggagaatccattgaacctgggaggcagaggttgcggtgagccaagatcgtgccactgcactgcagcctgggcgacagagcaagaccccgtctcagaaaacaaaaaacaaaaaaaaactacatgtggtccgaatgaaacaaaactaagcttagggtttaggaataatctgagaacacataagaattgtaggttgagcctagtagaattaaataggccccaagctggactggattcacccattcattcattcattatcttacttcctcaatgtgtccacgaatgccgggtgccatgggagaatataagaatataaataataaaaatatgtagtttctactcagaacttaaaattgagagagacagaatttacaggcaagtttaaataacatcaaagacagtaaaaatgcatatttcctaataatgacatgagcgagcgccaatgtaatagccttggcagtaaacgccgtgagttcagaagagtcacggtgagctggactagtcaggggaggcttctgggaggagggcccggagcgggacctgagagaagaacaggcagtgtgtctggaggatggaccaggaagggcagacccggagcctcatacagggtgcaggtacagaagctgcccccaggtgatgagctctcgtggccagaaccaccagctctagggaccagcccttgcgcgtatgtgcatcagccttcgtgtgtgctgttccctatgtctggaatggccgtcctctcccaaaccagctgcatttctcctcagggatgcctctgcctacaccactccttcccgcaccccacccgacccccaacgcccttcaccccagtcaccctatggcaatgatttattcatgtctgtcttcccttcccaggccatgaaccttgtgrggcagggactgtgttctacgcatttcttcttgaacccctttaccatttttgtgcctacggactcccagagtgctaaatcactcccaacagccccgcctatgcctctgccgggaccttttccaggggcagagagctggaagcacttggaaatttttctctcccacatcctcacatgccaccaccctcccactcccccagcccgcccccaggccttaaccaacggtggacaaatatgaaggtgtcagtaccccagccctccatgagacttagcttggttccactcatgtgcttgggtcccactttcccactccctttccactcctccccaccctcattacttttttttttttttaagacagggtctcactctgtcacccaggctaaagtgcagtggcacaatcataactcattgcagtctcaacctcctgggctcaagtggtcctcctgcctcagccttctgagtagctggtactatagatgcactccactcactgggctaattttttaatttcttgcagaaatgatgtcttgccatgttgcccaggctggtctggaactcctggactcaagcaatcttcctgccttggcctcccaaagcactgggattacaggtgtgagccatcatgcccagtcccctcattacttttatttatttatttatttatttattcaatttttgagacggagtctccctctcgttgcccagactggaatgcagtggtgtgatctcagcccactgcaatctccgcctcctgagttcaagcgattctcctgcctctgcttcctgagtagctgggattacaggcatgcgccactatgcccagctaatttttgtatttttagtagaaacagggtttcaccatgttggccaggctggtctcaaactcctgacctcaggtgatctgcccgccttggcctcccaaagtgtcgagattacaggcatgagccactgtgcctggcctatttatttttgagacagttctcactctgttgcccaggctggagtacagtggcacgatcacagctcactgaagcctggacctaagcgatcctcccacctaagcctcccaagtagctggatcacaggcgcatgccaccacgtctggctaattttttttgtagagattgggtcttactatattgcccaacctggtctcaaactcctgagctcaagaaaccctcctgcctccgcctctcaaagtgttgggattataggcgtgagccaccctgcccaacttctcattagttttaaataaatctcttttacttgaatctttgtctcagggcctgcttctggggaatccaacctaggatgcaaagtatttgctacacactattgcaactactttctactgcgcatgtgccatagggcactgttggtaaatgctctacagcttaagctctcgtttaatttgcataacaatgctatcatgatcatttcacagaagacagaaacaggcctagagaggtacagtgacccatgcaaggtcacacaggggacaaatggcagaactgggatttcaatttaggtctgtgctatgctaacaacactgattttaaccactacatcatcccagctctttttttttttttttttttttttttgagacggagtcttgctcttttcacccaggctagagtgcaatggcacgatcttggctcactgcaacctccgcctcctgggttaaagcaattctcctgcctcagcctcccacatggctgggattacaggcacccgccaccatacctggctaatttttgtagtttttttttagtagacacggggtttcaccatgttggccaggctggtcttgaactcccgacctcgtgatccaccagccttggcttcccaaagtgctgggattacaagcataagccaccgcgcctggcccatcccagctctttattcatctgtgtaaccctgacagagaatacagtgcctgggccgtcatgcacacttaatgtgtgttttgtgaaaggctaaattatttaatgaagggcccaattaacaaagagtagatcggaatgattggagtaaaataacccgaagaagagagagacatgttggagagacaggtcgggggaaaattagggaagatcttggtgccaagtgcaggagctcatatctgaaagtctctctcctctattagaactgtgcctgggcctgggcaacataacaagaccctgtctctgaacaaacaaaataagttagctgaacatggtagggcgcacctgtaatcccagctattccagaggctgaggtggaagattgcttgagctcaggaggtcaaagccagcctgggcaacacagcaagaccccatctctaaaaaaaaaaaaaattaaaattaaaaaagggccaggcacagtggctcacacctgtaatcctagcactttgggaggccaaggcaggaggatcgcttgagctcaggagtttgataccagtgtgggcaacatagtgtgacctcacctctacaaaaaaaatgtttaacatttggccaggttgccaggcgcagtggctcacgcttgtaatcccagcactttgggaggccgaggtgggcggatcgcgaggtcaggagatcgagaccacggtgaaaccccgtctctactaaaaatacaaaaaaaattagccgggagaggtggcgggcgcctgtagtcccagctactcgggaggctgaggtaggagaatggcgtgaacccgggaggcggacgttgcagtgagccgaggtcgcaccactgcactccagcctggacgacagagtgagactccatctcaaaaaaaaaaacaaacaattagccaggccatggtagtgcatgcctgtagtcccagctactcagcaggaagatcacctgagcatgagaggttgaagctggagtgagatatgattgcaccactgcactccagcttggatgacagagctgtctcagaaaaaaaaaaaaaattgtgcctagggtggggagaaacacatacatctctgggtatactgtggcaggaagctaaggatagaaaggaagaaggaggtctggacccctcaaactgaccctcaagccaataacgtggaattagttaggaggaaaaaaaattaattaattaattaattttttattttttgagacaggttcttgctctgtcgcccaggctggacagtgcagaggtgcagttacagctcactgcagccttgacctcctgggctcaagggatcctcctaccacagcgtcctgagtagctgggaccacaggcatgtgccaccatgtccagctaagagaaattcttaaagaagagagaaaggagggaaaggaactgagcccctgatgttgtctagggaaaaagctggggctctttacagcatgctgccttctttaattccacagcactatgtgggtttccatgcctttatttccttggaaagtatgagcttcttgaagacagcaactgtgccttgtctttctttgtatcccttccttctctcctagtacccagcctagaaggcactcaataaagcaaatgattagccccatttcacagacgaggaaccaacactgagagaggtaactcacctgtgcaagtcatatcacaagtgccaaagtcaagactggatggaggactgcctggctgcaaaccaattcttcccaggctgacatggcaggtaggtgagtgggaaagagaagggggaggcataaggcaattggagattttagtacctattaataggcagtggattttggcactcaaacaggctgtcttcattagctggggaggagactgagtgggcctggatggtatggaggtatttgcacagggaaacccattgtgctggcttatccattcagattagacaatgctggttcctctctacctgccttggctaagctcacctaggagtaaatgccccagggacaccgtcacgtctatgtcaacacagagtcacggaattaaataacagaataggatcacagatttacagaacaatagcccagaaccttggacatgacagatagttattaaatgcttggccaatgaaaaaaaaagaacctagaactagtatcacggtaaaatctaattatacaagctaagttaccttgagaaagcaccaggcacagccctgagccttgggcagcgaatgatgtctttggactagacagaagagatggttgtctgccctgctttgaagctctctggccagggaaactccaaaccattcatttgttcatccatttgcccacacaatcaacattcattgagcatctgctctgtggggtgctatgtgatggtgacagtcccaggaagcagtttcagtcctccctgccctcaaggggctctgtgtttagggaggacaaacatatacatcatgacaataaaatttgataaaaagttaaattagagagggggcaaaccctgacgtaggagcccaggagggactcctaacttctctgccaatttcatgtttcaaaattattagacccaagactcgctttggtataaacttaagctctcttactgtacatttttcttctcctgctaatctttaattaagtgcccaccaggtgcctggcactgaatcaaaactcaaaaaacttgctgaattaagccaaatgcacctcctgtgggtttttccccctaatatcctcagaggcagtaatcaattccctccccaaattccccagtccccaccccatccccacctttccttttgcagtataatctcaactcctgtgatgggggcaggcaggatagcatggtagtgagatcaacagactgtgacttgggttcttgtcgctacttagccattcattctgtatgtgaccttgggatgggactttagggggatttctttaataacctctaaactcccgtgcaatgctgagagccaaggtagcggctctcaggtcttgttccaagaactgccaccagagggcagcctagagactcctctcaggtgttttcctccagagcctttgctctttccctcactaatgtcatccactccctgggtccaccatcaaggcacacaggtgtccctttagccgtcaaggtgaccgttctaaggtgagccaggcatgtgaggcgaggcgagacaggctctgaagccctcaggaaaattcaggcacaggctgcttgtccaagtggacctcacgatcatcatttacacattctctccctgattatttcatgagccagcggtctacaggagaggataccacagccagtcaaagggatcagggccctgccctcagggagctgccttccagtgagggaggagagagatacacagatgcttacaaggtatctgtagtaggatgtcacctagtgatgaatggtgtgacgcagcttaggcagagcgaggaaatagggatggtcccaaacgtgtgtgtgtatgtgtgtttttttgagacagagtttcgctcttgtcgcccaggctggagtgcagtggtgcgatctcagctcactgcaacctctacctcccgggttcaagtgattctcctgtctcagcctcccgggtagctgggattacaagtgttcaccaccacacccggctaatttttgtatttttagtagagatggggtttcatcatgttggccaggctgggctacgagcgaaactccatctcaaaataataataataataataataataataataataataataataataataattcctgtctcctagggtcattgagagaatggagatcagtcctgcacgggcagacctcggcaccgagctgaatgttacacactgccaaggagtcggcgccggtctctggtgaagatgttctagtcccagggcccaagagaacagagcgggacacgagatgcctctctaacaggagtctgtgtttcctggtcacctctgtgtgctgggtgatgcctcaggtgccagggagacactgggcagtaggagaagctccctcctgtgaggactctcacccagaggcactcagacagcccagaacacaggagggaagatgggccaccacagtcccattcctaggcgctccaagggccagcccagcctagaagtccctcctggtacaagtctttccggcctctcctctcccgagagcacgttcagactgcaccctggcgctctctttcaccctgtcgaggaagctgtgactctaattctgggtgaccagtaacaataaaaaccactaatagccctagtggaaagaatgactatttaacaggtgcacataatgagcagacaaggagagctcttttccctcacactcactccgcaggacagagaggggaacagtcttgctactgtgacagctaatcacagcctctgtgtgcctccctcctcgcaagataagaggggtggcctcagggtgtgacggaaacagccaggaaggcttcggccagggagcgactgccctacagctgactccctgctgcttttgggaagtgggggtgaagggaaaagactcaggggagcggggggaagaaagcttggtgggcagaaccccacttgccaaattcctcctctttcaggattcagtggccaaaggtgagcctggctctggccccagtcttcactcccaagtgccggctgcagtgccctgggcccgtcggaggctggcagggctgctgggagggtgaggacaggcatgggaagagtggaaagtgggggctgacccagccaagctccaggaggccattccatagaggagagagccattggaccaggagccaagcagggaagaaggaaggaaaggtagagccgagcaccgcccacaccaggaataatctcaggaagaggatggtgtggacaggccccaggacagggcttctcagctaggggcaactcgttatgctgcagtgggggcacctaacagaaccccctagggtttttttaaagtatacagcacggtcctgcccaccactggcatcagatgaatgccatgtccaggggaaaccccagtacgtcctcatcacagccatgtgaacactgctgtttgttcataaaaatgtgttgcatccctagggggaaataaaaggagctaatccaatagagagaagagtggctcaggggagacagtcacccgcgaggaatgagggcaggacacacaggcccaggccgagattgcacagggtgttgcatgagtggctgtgtcagagccaagtgcacatgcccactgccacctccgccaaggccatcagtgaatccctccagcacctacaggggctttgcacataagagctgtgcttgcagggggtggggggtgggcacagggcaggaaaccagagaggagggcaccatgtccctgagtaaggtagaaaggggaagcacagcctggacacattcttccattcttttatttcaaaatatcaccaagtgcctgccacagctatgtgctgggaactcagtgaacgcacctgtgctttgcccactggcatggagcccacggtccgggggtgatggtttctgttgcatttcaaagccactggggcgctgcatagatggctggctgcagagtctatttgagacgaaggcaagaagtcagggcccatggactgtagattccacccttgatcgtctactacaggaaagaagccacagaagtgagggggtattcccaacaggaaggcagagaaggagagattaaaactgagtcaagaagtgtagccagtcccatgggagcacctgggctgaggagggtcagtccccgcagagatgttcggaacacctgattggggatgtgccctctccttctatgttcttggtcaccagaggaaccaggtgaagagtgtggatgactcagtccacccctccccgcacaggaaaaacaactcagaggacatcccctactgggagtgggccaggagcgctggcttccacgccaaggagaggagggtgttgctgtggctgcagaggtgagtccacagggaagcgtgggtgtcatggagaaagagagaggagaggaaatggcctctcgctggagccaagcagagaggaacaaaggaggggaggagcccacaggccctggagccccagagcagctctgggtggcccctttccttctctcctcagcacaaaataagctgcttttccaggctttttctcttcctcgtgctcactccattttccttcttccaagcctaagccctggtctaccaaggacccctatgtgaaacacttgtgctagaacttctgtcctgactctcaggcccccagaggaggaaagctgtggaggagggacctcatgagccagtaaccttcacttaggaactcttcacttttttttttttgagacggagtctcactctgtcacccaggctggagtgcagtggcatgaaatcggctcactgcagcctccgcctcgctggttcaaatgattctcctgcctcagcctcctgagtagctgggattacaggtgcctgcccccgcacccggctaatttttgtatttttagtagagatggttttgccatgttggccaggctggtctcaaactcctgagctcaagtgatccacctgccttggcctcccagagtgctgggattacaggcgtgagccaccatacctggccagctttgacagcagaaatcttctctctagaaatattcatatacataaggaattttgcacacatccttacagggtttagggagcctcttaagcctgtccatgaacctctgccattcatttatagcatggtgtcagctgcagagtaataggatctatgtgggcctggaagtccaacaggcctgggtttgaatctttttcaaacatttttgcttgttagcagctggtttggacaaataatttcaacttagttttctcatctgtgaaacagggggaaaaagcacttaccctgtagggctgttgtgaagattgaacaagacaactcatgttaaatacccaggcaccgtgtccgcctgcagcaggcagtcagtacatggtggatctcgtgccccaagcctcctctttatggagtccctcaagttgccccagctggcctcggattcctcccacctccacctcctgggtagctgggactacagcacgccatgcttcagccccctttgtgatcattcaaagccgtttggttccctgcaccctctcacatttactgcaattcctatagaatccaaattcttttaacctggtgttgtaggtcatctacaatcccgatccaagcagcctgtcccttctcccacaccactgtcctccacatgcatgtcccacaccaacccacgccatccgctggttctaaatacacctgaagcaccacaaggccccacgtctggctgcctgctctgtgcttctctcatcattcctcaaggcccggggaaagtcctgccacctccctgaagccaagtgcctacccgcttctccactctaggctgctctcttacgctgtgtgtccccagctggatggcagctcctcagggcaaggaacaatttctcttctttctcagtgcaacttccaaaacacagcagaggagcgtgcactgggggcaaaggctggcacagcaaacacagcagcacagggctgctggaggggtggaggggaccggaggggacagtgacctgcctgcttcataatacatggctgggagccccaccttttgcctatggaagccatgtgctttgagcttgattttctcgggtgtagagtgtctacgccacccacactttctcacccggtccaatgcacgggcacgggcaggggcagggccagaatctgttgctgtagaacacagcagagctgcctgcagtactgtggacgtcaccaaactggcagcttcactgaaggtgaccctggcctgtgtgggcactcagctgatgcccttctcttccctcctaagccccattagctttaggtcttcaccacctgcactgagagaaagattaatggtgagcaccagagtaaaatctagcacagaaggcagggtttcagtaaaaaaccaaaagcatggcagattagagctcaagggcccacaggtgggcaggacagtagaggaactgggcagcccagggtggcaggatggggtggacaggagtggacaggagagagtagccaggaggagggacgtggctgcaccaccagtccacgtgggcggctggagacctggcaggaggccaggagagactgtgcctcctccagcctggctgtcttggccaagttctgatggcagccaggaggactgtgagaaagaaaacaagacaaactttgctacctctggctggtcaggggaagtttgtgttggaaaagaagaagccacagattccactgttccgctgccttctcccctcacaccctgggacggcccagcaccttctagtccactcctccctgccctctgccactcccctcactaccccagtgccccctccctccttgcccaggtcctccagggagccaagcctgagagtctagggaggcccacaagaggaaatgatgttcggaaagagcagcatcactttattttttgagatggagtttcccttttgtcacccaggctggagtgcaatggtgcaatcttggctcactgcaacctctgcctccagggttcaagtgattctcctgcctcagcctcccgagtagcagggattacaggcgcccaccaccacgcctggctaatttttttgtatttttggtagagacggggtttcgccatgttggccaggctgatctcgaactcctggcctcaagtgatccgcccgcatcggcctcccatagtgctgggattacaagcgtgagccaccgtgcccagccgagcagcatcactttaaatggtatctctgtctcacatttgtgcctgcacctctatacaaggcaggaagggagcagaagacactctgctagcattggctgggctggagactcaggaaagggagcctggggctgtgggggtgaagctggcctatttgtctcccccagtggaaggatgaagacaggagcacctgcagggctccatttgctgggcttcctactgggggactgcaccccttgtcccatttaacctttttgataatttcccatttgatgttcacaaaaaccttgcgaaataagtgttatttaaaatacccccatttcacagatgaggagatggaagttcagaaaggtttgaagagccgcccaaagccaagaggttagctgggacttgaacccacgtcatcttcattctgaagccagtgttttttgtttgtttgtttttcccaaggcattagcccccttcaggggaccctgtcctatcccttcctctctccacagctccctcagcccctctttcctcccactcccattctgagtgtcactagcaagtcaagagcaagactagatggcagccactagctgcacgtggccaaccacatcattacaattaattaaaattaaagctccaaatctttggtggttaaacttgctccatgtggccagtggctaccgtatcggacagcacagacaggaaacatttccatcctcctgctggtctgaacttaaagggcggtcaggaggtagagggaatgggagagaccagcccaaggggcccagggacagagctgcctttgtgaggagatgaggtttgggaagtgagtttgggtgaggggagcaggggtgtctgaggcctgggggaggagagggcagcttgctggacttgcaggcacctccctccgatactgatactcccgcaatactagtgagcaattaattggctggggcggggcaggggccagagcaggagggaggccaacgcaatggaggagaagccaccaccctctcagcagatggcagggctccggttctcctggcctcctttcatcctttgaggacccgggagcctgcctggccctcctcgacctgcccagagctggctgctgcttcctgctcgctcatccagcccagggccagcagatgggctacggggccaccaccttcccaattagccttcttagcctcttgctcacccttgaggtctgggttaccagccatggggaggggctgtgagtggcctgtgagtaaccaaggcccagctttgacagaggtcatcagcccaggcccctcccctggctgagccctggctcccagcacccccccccacccccgccaccatgccgtccccagaggcccccagaagcgacaggtcagggagttggttttgggaatgtggtgccccatttctgagaaggagaaggttccccaggtgatggaacacgtaataggtatgtggctggcctggtacttggggagtggtgggagggggagcctgggggatgggggcagaactcatatttgaaggaggaaagtggaggtggagtgatggggtcctaaggaaggagcagatcaggggctgtgaatgggacctcattggggttgggtgggtggggctggagcgggcacccaagtgtggaagaaaaagctgaaacacccacgactgcgagtggagggcagatggagagacaggccaagccacggtaggcaggagagttaaggagccaggcagctgggtcccgtggcaagagtggccgccccagagtgggtggccgtggggcagagcgcctggttccgggttaggcaatgaggagccggggccaggcctgtcaggtggcaggatcgttagagccccgtggccatgggtaccccacactgcagccactgctgctgctgagtaggcagatgcaccgggctgattaccacgctcctcccggccacaccaacttcccccggggcacccaccccctccacctctcctcctctccccacagtgactcctgcccagggaatgtccagctctggcataaaggacccaggtgtcctcgagctgccatcagtcaggaggccgtgcagcccgagatgggctcgtctcgggcaccctggatggggcgtgtgggtgggcacgggatgatggcactgctgctggctggtctcctcctgccaggtaggaggctgggggccctgggaacaggagggaggcgggagggagactccgggagaggacccagcgaaggggacgggcaggggctctggaatctgccttttgagtctgggggttgctcctcactgtatggtcgcctcaggtaagtttcttaaacttcctgagccccagtttctgaaattctgaagtggggttaatgacacctacctctagtctgtgtgtctcaaattaaataatgtatgtgatatgtactttggaaattctagaggtttatataaatggtggtggtgatttttattatgggagcactacaagataatgattggacatttaatagtaataatatcatttttagagcctttttatatgctagactctgttttaagcacatttggattatatattagaacttttatttttattttttttgtgagatggagtcccactctgtctccaaggctggagtgcagtggcgtaatctcggctcactgcaacttccacctctcaggttcaagcgactctcatgcctcagcctctagagtagctgggacaacaggtgcccatcaccacacctggctaattttcttttttttgtatttttagtagaaacagggttttaccattttggtcaagctggtcttgaactcctgactcaagtgatccgctcgcctcggcctcccaaggtgctgggattacaggcatgagccaccacacccggcctatattagcacttttgatcattacaagaacggtatgaaaagagatttgctatttccactctacagatgaggacactgaggctcggagaggttaggaaactagctcaaaatcatgcattagaaggcagcaaagccaagatttcaaccccaggccaggcaacccctggacctgtgttgttgaccaccgggtacttatagcccttgaggaatttctgcgaccttcccatggtctagtgggtggttggtgtctgagggaatagcgaaagagagaggcaatgcatggtggattcgtgcagaggactgaagggaattggcacagctggggttcggcgtggaggtgcatgcagagaatttctttctgaggagagaacagggacatcacagaggatggcagtctggttgttggtggagggatcaggatgagtggcagtaataattcataatatataatgctttacactttctaaaacatctggccgcacatgatagcttgtgcctgtaatcccaacacttcaggaggccaaggcaggtgaatcgcctgaggtcaggagttcaagaccagcctggccaagatggtgaaaccccctctctactaaaaatacaaaaaattagctgggtgtggtggcgggcacctgtggtcccagctacttgggaggctgaggcaggagaatcgcttgcaccaaggaggcagaggttacagtgagctgagaccgtgttattgcactttagcctgggcaacaagaaactccatctcacaaaaaaaaaaaaaaaaaaaaaaaagaagaaaaaacttccaggtggatgatctcatttagttttcttcatagtaatgctgtgggaaggcagggaaaatttggcccctctgaatgtataaactaaagctcagagaggttcagtaacttgctagtatgtggctctgtttgtaacacgtgggacctggaggggctagggaaggcagaaggaacgcaggtgaaagagtcatggaggaaccatggggtaagttgggcctggggttttgagcaaaggaaaggaaagataaggaaagatgtggctccacatccctgagggaagtcaaggcagcagaagtcagatgaggggctggacagaggcaggtgtgctcagagagggaagctgattgtggccaggagcctcggaggttcgtggggtttcgtcctggttccctgggctgggccagcgagagcagggctggctcagggtgcggtgtcctgacacactggtaccagcaggttctgaagcaacaggtagtgaccccacatcctggcccccacccagctttactggcatggccagtgctgagataggaaatagggtttccattcctgaccccagcctgggctctcacgaagaagctggtgaccaaatcttagtcctcgagtgccctttcctttatttcagcccctctgcccccagctttgtctttttccagtgtctccttctatatgtgtctccacttctcagccctccattgttttgccttttgtcttcttccctctggtcccactgtctggcccaggatttttcccctaagaatttacgcctggactcctcagagcctcagtttccccaattctctgtctcttcagggtcctttcttttagacctatttgttcctgccccttctccattccctcttctttttaaaaaaaattttaattaaaaaacaaaatacagatggggtctatgttgcccaggctggtcttgaactctggggcgcatgcaatcctcccacctcagcctcccaaagtgctgggattaccggcgtgagccactgtgcccagccccctcttatattcaatgtattcctttgaggtcactcactttggcacgtaattttctatttttctggttggtgtttgcccacccttcccaaacaaagaaatgcctttattcggccacctcaatatcctttagagacaatagccagttcttcctcctttctccatccctaaactctccctgcgctctgcttgggagaaacccgagaggccgattactgagataaggcagaaaggtgagggaggaagccaagcctctttggcccttactaaccactgctttcctccacagggaccttggctaagagcattggcaccttctcagacccctgtaaggaccccacgcgtatcacctcccctaacgacccctgcctcactgggaagggtgactccagcggcttcagtagctacagtggctccagcagttctggcagctccatttccagtgccagaagctctggtggtggctccagtggtagctccagcggatccagcattgcccagggtggttctgcaggatcttttaagccaggaacggggtattcccaggtcagctactcctccggatctggctctagtctacaaggtgcatccggttcctcccagctggggagcagcagctctcactcgggaagcagcggctctcactcgggaagcagcagctctcattcgagcagcagcagcagctttcagttcagcagcagcagcttccaagtagggaatggctctgctctgccaaccaatgacaactcttaccgcggaatactaaacccttcccagcctggacaaagctcttcctcttcccaaacctctggggtatccagcagtggccaaagcgtcagctccaaccagcgtccctgtagttcggacatccccgactctccctgcagtggagggcccatcgtctcgcactctggcccctacatccccagctcccactctgtgtcagggggtcagaggcctgtggtggtggtggtggaccagcacggttctggtgcccctggagtggttcaaggtcccccctgtagcaatggtggccttccaggcaagccctgtcccccaatcacctctgtagacaaatcctatggtggctacgaggtggtgggtggctcctctgacagttatctggttccaggcatgacctacagtaagggtaaaatctatcctgtgggctacttcaccaaagagaaccctgtgaaaggctctccaggggtcccttcctttgcagctgggccccccatctctgagggcaaatacttctccagcaaccccatcatccccagccagtcggcagcttcctcggccattgcgttccagccagtggggactggtggggtccagctctgtggaggcggctccacgggctccaagggaccctgctctccctccagttctcgagtccccagcagttctagcatttccagcagctccggttcaccctaccatccctgcggcagtgcttcccagagcccctgctccccaccaggcaccggctccttcagcagcagctccagttcccaatcgagtggcaaaatcatccttcagccttgtggcagcaagtccagctcttctggtcacccttgcatgtctgtctcctccttgacactgactgggggccccgatggctctccccatcctgatccctccgctggtgccaagccctgtggctccagcagtgctggaaagatcccctgccgctccatccgggatatcctagcccaagtgaagcctctggggccccagctagctgaccctgaagttttcctaccccaaggagagttactcgacagtccataagtcaactgttgtgtgtgtgcatgccttgggcacaaacaagcacatacactatatcccatatgggagaaggccagtgcccaggcatagggttagctcagtttccctccttcccaaaagagtggttctgctttctctactaccctaaggttgcagactctctcttatcaccccttcctccttcctcttctcaaaatggtagattcaaagctcctctcttgattctctcctactgtttaaattcccattccaccacagtgcccctcagccagatcaccaccccttacaattccctctactgtgttgaaatggtccattgagtaacacccccatcaccttctcaactgggaaacccctgaaatgctctcagagcacctctgacgcctgaagaagttataccttcctcttcccctttaccaaataaagcaaagtcaaaccatcatctggaaacagtggccacttttcactgacctctcttcgacatctagtcaacccacccaatatgccactgggctttcgctcccaattccaccccaccctccattacagagctcaccacgccctcctagatcaccgtccccaacacacccattgcctctcaaggcccttatctcagccccttcctgtggccatttccctcagtgcccagatgattccctgggtgagggagacactggggcaccctcagaggttggagcaggctccctgctgtccctggatcctggacagatggctcagtaaactgtggggactaggtgcagactttttgccttcttggagtcctgggtctcctctgagagtctgggtggtgctcttcctacgcctctagaggtctctgtgtccctcattttccttcaaaagcgggctgtgtttctcttctaccttccagctcctcccacagaggaggaagacaataaatatttgttgaactgaaagcagagattgcctggcctcccagatccttccgccatttccctcctctctcattgctccaggaaatccattctcttcccattcctcattcaccgtggggtcccccttccccttatttagggccctcagtgttttctctccctcccctcccctcccctccccacccaaactccttttcttccaccattagcattcctcaccttctagatgccatcctctctgggagtcatgagtctcgatttcctgggtttctgggacacctggaagcttgggaaggctgggacacaacaactccaaccagattcctgtcagctgagtaggaggccagttgggcgttgttcctggagctgggggtggagagagtaaaggactgagaggatgggagcggggcagggagtgcagccaagcagggtgactcactggcctagatcaagaggcccagcctgtggcagaacagagctgccagtggtctctccatcttcacactccctgctctgctggggtccagagtgagagtgtgagcaacatggctctcaggtgagggctgagaaggcagagtgccccagtgggaaagaggagtcgcttccactggagaagagagagaaagtggagtgtgtggtggggtccatgcgacttaagtcctgagacaggcagggagaggctgaggcggacgaagttcccgcatcccaaggagggcagagtggattgtgcttgtccctgtaggagccccaccccccaccccaggccacctctcagagcctctgcttggctgcaaaggaattcacccctactgtagcacttaacccattccctcctatcagggtggtgctgtctggtcctgaatttagaactgttgaaactccaagtctggaatcagcaaaaatgtattacattgaccagaaagggattgaatcacccttggtccagcatctggcccctgatctgcagccaatggcaggaatcgaggtcctcagatgcttcatgaatgggaattgcagggagagaaggctctctgatgtggtgtttcctcgagtctcctgctgtgctccaaattaaaagcttgtgtaaaactcatgcatgtcatccaaaaaggcctctgggctccatccactgccagttctggagaggagctcttcactcctccagtggttaagccagcaggggcaggtggggaggacacagcagtagaatcagccaacagctcatgtttagaccttgggcagccagggaagcctactcctggggcctcccggaagccatggagagaacaaagccattgcatttttataataaaatttgcaaacatatttaaaagccaacaaactgttaatgaatctctacattctcatcgcccagcttcaacaaggatcccagcttcaacaaggatcaagtcctggccatttgacagcagcatttaaaggctctcctctactgttacttggaaatagccactttctcccaaggtttcttatactctgtggcacatctgaccaccagtagcaggcagaatgatgtcttcaaccccaacaccatcaaagatgtccacatcctaatccctggaacgtaggaattaggttacatggcaaagggaaattaaggttccagatgggattaaggttgctattcggctgacttcacagagattatcatggattattcaggtgggtccagtgtagtcaccaggtcccttaatgtggacatgggaggcagaagaggaagtctgagtgatacagtgtaagaaatggctgattttggctttggagatggaggaaggggaccatgagccaaagaacacaggatgcctctagaaggtgaaaaagcagggaaagggattttcccctgaggcccccagaaagaatcacagccctgctgacacctttattttaatccactgagacctgttttagacttctgatctccaaaactgtaaagtaataaatccatgttgttgtaagccattcggttcatggtaatttgtcactgcagcagcaggaattagtcagtatctcataaggatggcatccaggtccatttccctagctagatccagggtctcatgtaggagcagctcctcagatggggccacttctgcaccccagaacctcctgcaggttggggccaaggtgaaggagatatgaggatgcatgagaaaggggtgctgggaggaaacaatccagctcccaaaaagaaacaagtgtttctgttgctgagagaggcaattaagagagtgggaccccagggtggaggtccttgtgtatagagaagcagggctggggaggctggcaaccagggatgagctgtgagccaggacacctgggccaagaaggggcagggaggtcaaggaaaggagccagggcgggagacacccagcttcctctgggacattcattcaagtgacacctgttgccacagaccacattaggaatgagggtggaatgtggaggtttattgtcttcacaaccactagcccagcctgtttctgctgtcccccaccccactaccaggataaagggctggctgtcttggggctgagggagatcgggtgctgagcaggatgcagggccgcgtggcagggagctgcgctcctctgggcctgctcctggtctgtcttcatctcccaggtatggaggccgtgatgcccttgggcaggagggactggaggtcccccaggaaacaggaattaaggaaaggggtaaaggcaggagggtacacatttaggtccctgagggaaaaggaagaataggcataggggaagcaaagggaactggggactcggggactggagaccactggttgctttatcttccctttccctcaggcctctttgcccggagcatcggtgttgtggaggagaaagtttcccaaaacttggggaccaacttgcctcagctcggacaaccttcctccactggcccctctaactctgaacatccgcagcccgctctggaccctaggtctaatgacttggcaagggttcctctgaagctcagcgtgcctgcatcagatggcttcccacctgcaggaggttctgcagtgcagaggtggcctccatcgtgggggctgcctgccatggattcctggccccctgaggatccttggcagatgatggctgctgcggctgaggaccgcctgggggaagcgctgcctgaagaactctcttacctctccagtgctgcggccctcgctccgggcagtggccctttgcctggggagtcttctcccgatgccacaggcctctcacccaaggcttcactcctccaccaggactcggagtccagacgactgccccgttctaattcactgggagccgggggaaaaatcctttcccaacgccctccctggtctctcatccacagggttctgcctgatcacccctggggtaccctgaatcccagtgtgtcctggggaggtggaggccctgggactggttggggaacgaggcccatgccacaccctgagggaatctggggtatcaataatcaacccccaggtaccagctggggaaatattaatcggtatccaggaggcagctggggaaatattaatcggtatccaggaggcagctgggggaatattaatcggtatccaggaggcagctgggggaatattcatctatacccaggtatcaataacccatttcctcctggagtcctccgccctcctggctcttcttggaacatcccagctggcttccctaatcctccaagccctaggttgcagtggggctagagcacgatagagggaaacccaacattgggagttagagtcctgctcccgccccttgctgtgtgggctcaatccaggccctgtcagcatgtttccagcactatccccacttttcagtgcctcccctgctcatctccaataaaataaaagcacttatggaatttgcttctccttggtttctttgtttctgggcataagctgaagtgagtctgggcataagctgaagtgagtctgttcattcctgttttctagccatccccacggccctctaggggcccctgcagacgctgtcttgctatccccatccttcacaaaggatcagtgcccaagtgcttgagggtggagcctcagtctcaccccggccaggtgggagagctgttccagaattgtgctggaatctgaaagggggaggagggacagcaggactaattgagatggcacctgcagcagggggcaaggatgaggtcccagaaggcggctccagggccaggtggacaggattccttgcaactcacagaaacaggaagccaaaagtcgcaatgtctacgcttcacttgtcttttcttccccggaaagtcaagcttcttggaagtggaggtacatcgacctcctcccttacaggcatcatttagcacattgtgtcccacagaaccacagactttgaagagttgctgagtaaatagcagacctcgataaaggaaaagagaaaagggagaaaggaaagggagaaaaaaaccttgaagccaacaatcccacctggggtggcatttgatgctttcattcccaagtgatgacacagtctcagcctttggtcacagtattgtctctcctgccctcccttcggttttcccaggagctcaacatcctcacacaggagttggagtgacggcagcgaagggtcaggctacaaaagcacggaagaatcagcaggtgtgggttggaggtgatttgggtctggattctctcttccctgtgccatgcctgcagtg

SEQ ID NO: 5 - protein, 152 aa, Homo sapiens; identical to SEQ ID NO: 2

SEQ ID NO: 6 - protein, 63 aa, Homo sapiens
MTCTDQKSHSQRALGTQTPALQGPQLLNTDPSSKETRPPTLILTDFATWSQQTISGMQGTSKQ

SEQ ID NO: 7 - protein, 41 aa, Homo sapiens
MTCTDQKSHSQRALGTQTPALQGPQLLNTDPSSKETRPPPR

SEQ ID NO: 8 - DNA, 46 bp, Homo sapiens
cttctagatgccatcctctctgggagtcatgagtctcgatttcctg

SEQ ID NO: 9 - DNA, 160 bp, Homo sapiens
gctttgcccactggcatggagcccacggtccgggggtgatggtttctgttgcatttcaaagccactggggcgctgcatagatggctggctgcagagtctatttgagacgaaggcaagaagtcagggcccatggactgtagattccacccttgatcgtcta

SEQ ID NO: 10 - DNA, 77 bp, Homo sapiens
gtgcaagtcatatcacaagtgccaaagtcaagactggatggaggactgcctggctgcaaaccaattcttcccaggct

SEQ ID NO: 11 - DNA, 31 bp, Homo sapiens
gagagctctttgagagtgacttttttggtct

SEQ ID NO: 12 - DNA, 124 bp, Homo sapiens
agaaatggtttgctggctccatgtggcaaagtcggtcaggattaacgtgggggggacgagtttccttggagctgggatctgtgttaaggagctggggtccttgtaaagctggggtctgtgtgcc

SEQ ID NO: 13 - DNA, 421 bp, Homo sapiens
gcggaccaggcagggaacaacctgggaggaatcaaattttattttggacatgttacttctgaaaggctaacagacttccaggcagaaaggtccttgagggaaacattctaggggtctctctgggaggcttagatcaaggagctgagaccaaaaggagaatgggagggaggagacgagtacaatagagttggagccaaggtcctagaggcggataggtggattcctgagggaggaggaaggggctgaggttgctggagcctggcagcttcttccggagccattggcaggactgatgcaaacagctctgggtgggaagagggaaccaggatatcctcctgtgtccttccttttctgcagtcatcctgggtggctgccagatggaattccttggatatcattgcttggaggtcccctgcatgcc

SEQ ID NO: 14 - protein, 15 aa, Homo sapiens
MISKEFHLAATQDDK

SEQ ID NO: 15 - protein, 15 aa, Homo sapiens
MISKEFHLAATQDDC

SEQ ID NO: 16 - DNA, 22 bp, Artificial sequence
  primer
daggtgttccgaacatctctgc

SEQ ID NO: 17 - DNA, 22 bp, Artificial sequence
  primer
dacagcctggacacattcttcc

SEQ ID NO: 18 - DNA, 21 bp, Artificial sequence
  Primer
daagacagcctgtttgagtgc

SEQ ID NO: 19 - DNA, 21 bp, Artificial sequence
  Primer
dtgtatcccttccttctctcc

SEQ ID NO: 20 - DNA, 21 bp, Artificial sequence
  Primer
daaaggtaagaggtggtgagg

SEQ ID NO: 21 - DNA, 21 bp, Artificial sequence
  Primer
datctggctcaccagaaatgg

SEQ ID NO: 22 - DNA, 21 bp, Artificial sequence
  Primer
dtttcaaacctgggatgcagc

SEQ ID NO: 23 - DNA, 21 bp, Artificial sequence
  Primer
dagatgagatcacgccattgc

SEQ ID NO: 24 - DNA, 21 bp, Artificial sequence
  Primer
datgcctgtaaaggaggaagg

SEQ ID NO: 25 - DNA, 21 bp, Artificial sequence
  Primer
daaagtgggtcaagtgaacgg

SEQ ID NO: 26 - DNA, 21 bp, Artificial sequence
  Primer
dtaagctccatccacccctgg

SEQ ID NO: 27 - DNA, 21 bp, Artificial sequence
  Primer
daactggacgcatggggttgg

SEQ ID NO: 28 - DNA, 21 bp, Artificial sequence
  Primer
datgggatccaggcatcctgc

SEQ ID NO: 29 - DNA, 21 bp, Artificial sequence
  Primer
dtttggacagggtgtggaggg

SEQ ID NO: 30 - DNA, 61 bp, Homo sapiens
ccccaatcaggtgttccgaacatctctgcgrrgactgaccctcctcagcccaggtgctccy

SEQ ID NO: 31 - DNA, 61 bp, Homo sapiens
cccaatcaggtgttccgaacatctctgcgrrgactgaccctcctcagcccaggtgctccya

SEQ ID NO: 32 - DNA, 61 bp, Homo sapiens
rrgactgaccctcctcagcccaggtgctccyatgggactggctacacttcttgactcagtt

SEQ ID NO: 33 - DNA, 61 bp, Homo sapiens
gtagacgatcaagggtggaatctacagtccrtgrgccctgacttcttgccttcrtctcaaa

SEQ ID NO: 34 - DNA, 61 bp, Homo sapiens
gacgatcaagggtggaatctacagtccrtgrgccctgacttcttgccttcrtctcaaatag

SEQ ID NO: 35 - DNA, 61 bp, Homo sapiens
acagtccrtgrgccctgacttcttgccttcrtctcaaatagactctgcagccagccatcta

SEQ ID NO: 36 - DNA, 61 bp, Homo sapiens
attaataggtactaaaatctccaattgcctyatgcctcccccttctctttcccactcacct

SEQ ID NO: 37 - DNA, 61 bp, Homo sapiens
gtgagttacctctctcagtgttggttcctcktctgtgaaatggggctaatcatttgcttta

SEQ ID NO: 38 - DNA, 61 bp, Homo sapiens
cccagccccaggaggaggagcctgtctggayggacgcagcctgaactgacccacaaacaga

SEQ ID NO: 39 - DNA, 61 bp, Homo sapiens
taggtttgtaaatacttaactgatggtaaartgtcatgaacccctacccccgatggatctg

SEQ ID NO: 40 - DNA, 61 bp, Homo sapiens
gctttgtcctcaggccaacctgcaacccaargtgggttacaccttggcccccaggcacaca

SEQ ID NO: 41 - DNA, 61 bp, Homo sapiens
ccaggcacacagaccccagctttacaaggamcccagctccttaacacagatcccagctccr

SEQ ID NO: 42 - DNA, 60 bp, Homo sapiens
ycccagctccttaacacagatcccagctccraggaaactcrtcccccccacgttaatcct

SEQ ID NO: 43 - DNA, 60 bp, Homo sapiens
ttaacacagatcccagctccraggaaactcrtcccccccacgttaatcctgaccgacttt

SEQ ID NO: 44 - DNA, 67 bp, Homo sapiens
aacacagatcccagctccraggaaactcrtcccccccacgttaatcctgaccgactttgccacatgg

SEQ ID NO: 45 - DNA, 66 bp, Homo sapiens
aacacagatcccagctccraggaaactcrtccccccacgttaatcctgaccgactttgccacatgg

SEQ ID NO: 46 - DNA, 68 bp, Homo sapiens
aacacagatcccagctccraggaaactcrtccccccccacgttaatcctgaccgactttgccacatgg

SEQ ID NO: 47 - DNA, 61 bp, Homo sapiens
tctgcaccatgtcccccacccaatgtgtccwgaaagccatttctggtgagccagatgcacc

SEQ ID NO: 48 - DNA, 61 bp, Homo sapiens
catttctggtgagccagatgcaccttctgcrtcccctgaattcctgtccccaaccccatgc

SEQ ID NO: 49 - DNA, 61 bp, Homo sapiens
tccacctatccgcctctaggaccttggctcyaactctattgtactcgtctcctccctccca

SEQ ID NO: 50 - DNA, 61 bp, Homo sapiens
  misc_feature (31)..(31): n is c or no base
ctccttgatctaagcctcccagagagacccntagaaygtttccctcaaggacctttctgcc

SEQ ID NO: 51 - DNA, 61 bp, Homo sapiens
gatctaagcctcccagagagacccctagaaygtttccctcaaggacctttctgcctggaag

SEQ ID NO: 52 - DNA, 74 bp, Homo sapiens
gaaatagccacyttctcccaaggtttcttatactctrtggcacatctgaccaccagtagcaggcagaatgatgt

SEQ ID NO: 53 - DNA, 54 bp, Homo sapiens
ctcctctactgttacttggaaatagccacyttctcccaaggtttcttatactct

SEQ ID NO: 54 - DNA, 81 bp, Homo sapiens
gatcaagtcctggccatttgacagcagcatttaaaggcyctcctctactgttacttggaaatagccacyttctcccaaggt

SEQ ID NO: 55 - DNA, 66 bp, Homo sapiens
catgtttagaccttgggcagccagggaagcytactcctggggcctcccggaagccatggagagaac

SEQ ID NO: 56 - DNA, 76 bp, Homo sapiens
ctcttcactcctccagtggttaagccagcaggggcaggyggggaggacacagcagtagaatcagccaacagctcat

SEQ ID NO: 57 - DNA, 70 bp, Homo sapiens
aggcctctgggctccatccactgccagttctggagwggagctcttcactcctccagtggttaagccagca

SEQ ID NO: 58 - DNA, 80 bp, Homo sapiens
acattgaccagaaagggattgaatcacccttggtccagcrtctggcccctgatctgcagccaatggcaggaatcgaggtc

SEQ ID NO: 59 - DNA, 80 bp, Homo sapiens
tgaatttagaactgttgaaactccaagtctggaatcagcaraaatgtattacattgaccagaaagggattgaatcaccct

SEQ ID NO: 60 - DNA, 75 bp, Homo sapiens
ctcagagcctctgcttggctgcaaaggaattcacccytactgtagcacttaacccattccctcctatcagggtgg

SEQ ID NO: 61 - DNA, 76 bp, Homo sapiens
ggattgtgcttgtccctgtaggagccccaccccccacccyaggccacctctcagagcctctgcttggctgcaaagg

SEQ ID NO: 62 - DNA, 75 bp, Homo sapiens
tgagacaggcagggagaggctgaggcggasgaagttccygcatcccaaggagggcagagtggattgtgcttgtcc

SEQ ID NO: 63 - DNA, 76 bp, Homo sapiens
gacttaagtcctgagacaggcagggagaggctgaggcggasgaagttccygcatcccaaggagggcagagtggatt

SEQ ID NO: 64 - DNA, 78 bp, Homo sapiens
gctgagaaggcagagtgccccmgtgggaaagaggagtcgcytccactggagaagagagagaaagtggagtgtgtggtg

SEQ ID NO: 65 - DNA, 79 bp, Homo sapiens
aacatggctctcaggtgagggctgagaaggcagagtgccccmgtgggaaagaggagtcgcytccactggagaagagaga

SEQ ID NO: 66 - DNA, 79 bp, Homo sapiens
tagatcaagaggcccagcctgtggcagaacagagctgccrgtggtctctccatcttcacactccctgctctgctggggt

SEQ ID NO: 67 - DNA, 76 bp, Homo sapiens
tctcagccccttcctgtggccatttccctcagtgcycagatgattccctgggtgagggagacactggggcaccctc

SEQ ID NO: 68 - DNA, 71 bp, Homo sapiens
taccccaaggagagttactcgacagtccataagaagtcaactgttgtgtgtgtgcatgccttgggcacaaa

SEQ ID NO: 69 - DNA, 72 bp, Homo sapiens
agttcccaatcsagtggcaaaatcatccttcagccttgyggcagcaagtccagctcttctggtcacccttgc

SEQ ID NO: 70 - DNA, 74 bp, Homo sapiens
ggcaccggctccttcagcagcagctccagttcccaatcsagtggcaaaatcatccttcagccttgyggcagcaa

SEQ ID NO: 71 - DNA, 67 bp, Homo sapiens
ccagcrgttctagcatttccagcagckccggttyaccctaccatccctgcggcagtgcttcccagag

SEQ ID NO: 72 - DNA, 69 bp, Homo sapiens
ctcgagtccccagcrgttctagcatttccagcagckccggttyaccctaccatccctgcggcagtgctt

SEQ ID NO: 73 - DNA, 76 bp, Homo sapiens
ccaagggaccctgctctccctccagttctcgagtccccagcrgttctagcatttccagcagckccggttyacccta

SEQ ID NO: 74 - DNA, 75 bp, Homo sapiens
accccatcatccccagccagtcggcagcttcctcggccattgcrttccagccagtggggactggtggggtccagc

SEQ ID NO: 75 - DNA, 71 bp, Homo sapiens
ccaggcatgacctacagtaagggtaaaatctaycctgtgggctacttcaccaaagagaaccctgtgaaagg

SEQ ID NO: 76 - DNA, 81 bp, Homo sapiens
cagggaccttggctaagagcattggcaccttctcagaccyctgtaaggaccccacgcgtatcacctcccctaacgacccct

SEQ ID NO: 77 - DNA, 84 bp, Homo sapiens
kactgagataaggcagaaaggtgaggraggaagccaagcctcyttggcccttactaaccactgctttcctccacagggaccttg

SEQ ID NO: 78 - DNA, 73 bp, Homo sapiens
agaggccgatkactgagataaggcagaaaggtgaggraggaagccaagcctcyttggcccttactaaccactg

SEQ ID NO: 79 - DNA, 73 bp, Homo sapiens
ccctgcgctctgcttgggagaaacccgagaggccgatkactgagataaggcagaaaggtgaggraggaagcca

SEQ ID NO: 80 - DNA, 74 bp, Homo sapiens
tcaatgtattcctttgaggycactcactttggcacstaattttctatttttctggttggtgtttgcccaccctt

SEQ ID NO: 81 - DNA, 73 bp, Homo sapiens
agccccctcttatattcaatgtattcctttgaggycactcactttggcacstaattttctatttttctggttg

SEQ ID NO: 82 - DNA, 74 bp, Homo sapiens
tcttgaactctggggcrcatgcaatcctcccacctcrgcctcccaaagtgctgggattaccggcgtgagccact

SEQ ID NO: 83 - DNA, 76 bp, Homo sapiens
gggtctatgttgcccaggctggtcttgaactctggggcrcatgcaatcctcccacctcrgcctcccaaagtgctgg

SEQ ID NO: 84 - DNA, 74 bp, Homo sapiens
aaaaaaattttaattaaaaaacaaaatacagayrgggtctatgttgcccaggctggtcttgaactctggggcrc

SEQ ID NO: 85 - DNA, 74 bp, Homo sapiens; identical to SEQ ID NO: 84

SEQ ID NO: 86 - DNA, 67 bp, Homo sapiens
ctgtctcttcagggtcctttcttttagacctayttgttcctgccccttctccattccctcttctttt

SEQ ID NO: 87 - DNA, 72 bp, Homo sapiens
ggaggaaccayggggtaagttgggcctggggttttsagcaaaggaaaggaaagataaggaaagatgtggctc

SEQ ID NO: 88 - DNA, 68 bp, Homo sapiens
cagaaggaacgcaggwgaaagagtcatggaggaaccayggggtaagttgggcctggggttttsagcaa

SEQ ID NO: 89 - DNA, 75 bp, Homo sapiens
ctggaggggctagggaaggcagaaggaacgcaggwgaaagagtcatggaggaaccayggggtaagttgggcctgg

SEQ ID NO: 90 - DNA, 164 bp, Homo sapiens
aggtgttccgaacatctctgcgrrgactgaccctcctcagcccaggtgctccyatgggactggctacacttcttgactcagttttaatctctccttctctgccttcctgttgggaataccccctcacttctgtggcttctttcctgtagtagacgatcaagggt

SEQ ID NO: 91 - DNA, 86 bp, Homo sapiens
aggtgttccgaacatctctgcgrrgactgaccctcctcagcccaggtgctccyatgggactggctacacttcttgactcagtttta

SEQ ID NO: 92 - DNA, 71 bp, Homo sapiens
tctctgcgrrgactgaccctcctcagcccaggtgctccyatgggactggctacacttcttgactcagtttt

SEQ ID NO: 93 - DNA, 84 bp, Homo sapiens
tcaagggtggaatctacagtccrtgrgccctgacttcttgccttcrtctcaaatagactctgcagccagccatctatgcagcgc

SEQ ID NO: 94 - DNA, 80 bp, Homo sapiens
gggtggaatctacagtccrtgrgccctgacttcttgccttcrtctcaaatagactctgcagccagccatctatgcagcgc

SEQ ID NO: 95 - DNA, 82 bp, Homo sapiens
atctacagtccrtgrgccctgacttcttgccttcrtctcaaatagactctgcagccagccatctatgcagcgccccagtggc

SEQ ID NO: 96 - DNA, 80 bp, Homo sapiens
cctattaataggtactaaaatctccaattgcctyatgcctcccccttctctttcccactcacctacctgccatgtcagcc

SEQ ID NO: 97 - DNA, 89 bp, Homo sapiens
ggcacttgtgatatgacttgcacaggtgagttacctctctcagtgttggttcctcktctgtgaaatggggctaatcatttgctttattg

SEQ ID NO: 98 - DNA, 83 bp, Homo sapiens
cagccccacccagccccagccccaggaggaggagcctgtctggayggacgcagcctgaactgacccacaaacagaccaaaaaa

SEQ ID NO: 99 - DNA, 114 bp, Homo sapiens
accaaaaaagtcactctcaaagagctctcggtaggtttgtaaatacttaactgatggtaaartgtcatgaacccctacccccgatggatctgaaccgttcacttgacccacttt

SEQ ID NO: 100 - DNA, 81 bp, Homo sapiens
cactagctttgtcctcaggccaacctgcaacccaargtgggttacaccttggcccccaggcacacagaccccagctttaca

SEQ ID NO: 101 - DNA, 131 bp, Homo sapiens
tcaggccaacctgcaacccaargtgggttacaccttggcccccaggcacacagaccccagctttacaaggamcccagctccttaacacagatcccagctccraggaaactcgtcccccccacgttaatcct

SEQ ID NO: 102 - DNA, 118 bp, Homo sapiens
tcacagaccccagctttacaaggamcccagctccttaacacagatcccagctccraggaaactcrtcccccccacgttaatcctgaccgactttgccacatggagccagcaaaccatt

SEQ ID NO: 103 - DNA, 60 bp, Homo sapiens; identical to SEQ ID NO: 43

SEQ ID NO: 104 - DNA, 68 bp, Homo sapiens; identical to SEQ ID NO: 46

SEQ ID NO: 105 - DNA, 97 bp, Homo sapiens
agccaaatgcaccttctgcaccatgtcccccacccaatgtgtccwgaaagccatttctggtgagccagatgcaccttctgcrtcccctgaattcctg

SEQ ID NO: 106 - DNA, 103 bp, Homo sapiens
gcaccatgtcccccacccaatgtgtccwgaaagccatttctggtgagccagatgcaccttctgcrtcccctgaattcctgtccccaaccccatgcgtccagtt

SEQ ID NO: 107 - DNA, 89 bp, Homo sapiens
tcctccctcaggaatccacctatccgcctctaggaccttggctcyaactctattgtactcgtctcctccctcccattctccttttggtc

SEQ ID NO: 108 - DNA, 85 bp, Homo sapiens
cctcccattctccttttggtctcagctccttgatctaagcctcccagagagacccctagaaygtttccctcaaggacctttctgc

SEQ ID NO: 109 - DNA, 84 bp, Homo sapiens
attctccttttggtctcagctccttgatctaagcctcccagagagacccctagaaygtttccctcaaggacctttctgcctgga